(19) United States
(12) Patent Application Publication
(10) Pub. No.: US 2007/0059720 A9
(43) Pub. Date: Mar. 15, 2007

(54) RNA expression profile predicting response to tamoxifen in breast cancer patients

(76) Inventors: Suzanne Fuqua, Sugar Land, TX (US); Yukun Cui, Sugar Land, TX (US); C. K. Osborne, Houston, TX (US)

Correspondence Address: Fulbright & Jaworski L.L.P., Fulbright Tower, 1301 McKinney, Suite 5100, Houston, TX, 77010-3095, US

(21) Appl. No.: 11/295,188

(22) Filed: Dec. 6, 2005

Related U.S. Application Data
(60) Provisional application No. 60/633,632, filed on Dec. 6, 2004.

(51) Int. Cl. C12Q 1/68

(52) U.S. Cl. 435/6

(57) ABSTRACT

The present invention regards predicting a response to a therapy using RNA expression profiling. In particular, a resistance to a chemotherapy, such as tamoxifen, is predicted by comparing expressed genes in a patient on the therapy to a patient sensitive to the chemotherapy. In further embodiments, there is an RNA expression profile indicative of tamoxifen resistance in an individual. In additional embodiments, the RNA expression profile comprises DUSP6 EBP50, and/or RhoGDIa.

The present invention claims priority to U.S. Provisional Patent Application Ser. No. 60/633,632, filed Dec. 6, 2004, which is incorporated by reference herein in its entirety.

### STATEMENT REGARDING FEDERALLY SPONSORED RESEARCH OR DEVELOPMENT

The present invention utilized funds from the Department of Defense Grant DAMD 17-99-1-9399; Department of Defense Breast Cancer Concept Award, Contract W81XWH0410640; and the National Institutes of Health Grant R01-CA72038. The United States Government may have certain rights in the invention.

### FIELD OF THE INVENTION

The present invention concerns the fields of molecular biology, cell biology, cancer therapy, and medicine.

### BACKGROUND OF THE INVENTION

Breast cancer remains a significant health problem in the United States, affecting the lives of over 140,000 additional women each year. Breast cancer treatment involves surgical removal of the tumor, followed by adjuvant therapy to eradicate malignant cells that may have escaped from the site of the primary tumor. Since the steroid hormone estrogen can stimulate breast tumor growth, agents that either inhibit estrogen synthesis, or antiestrogens like tamoxifen (Tam) which block its receptor, are the standard therapies offered to women with estrogen receptor (ER)α-positive cancer (Baum et al., 2002). In many cases, however, these therapies eventually fail and metastases appear as endocrine-resistant disease. It is hoped that a thorough understanding of the factors that stimulate breast tumor growth during the transition to resistance will afford new strategies for inhibiting metastatic spread of this disease.

Prediction of Clinical Course

The standard prognostic factors currently used for primary breast cancer decision making in the United States (reviewed in (Clark, 2000)) are: involved axillary node status (Fisher et al., 1978), histologic subtype, tumor size (Carter et al., 1989), nuclear grade (Scarff and Torioni, 1968; Fisher et al., 1980), estrogen and progesterone receptor (ER and PR) status (McGuire et al., 1992), and measures of cellular proliferation (Clark, 2000). A number of factors useful for prediction of treatment outcomes have also been put into routine clinical practice. These include: ER, PR (McGuire, 1978), and HER-2/c-ErbB-2. Although many genes were originally attractive biomarkers with appropriate biologic rationale, they have failed to independently improve the prediction of outcome when compared to these standard factors. In addition, while combinations of standard prognostic factors can identify subsets of patients with highly significantly different disease survival curves, they still predict individual outcomes poorly. Thus, few molecular markers discovered during the current revolution in breast cancer molecular biological studies have come into clinical use as standard prognostic or predictive factors. In addition, the role of prognostic factors in the management of breast cancer has clearly changed, with the majority of node-negative patients now undergoing systemic adjuvant therapy because one cannot precisely determine an individual's risk of recurrence. Undoubtedly, since only a minority of node-negative patients will ever develop a recurrence, there is a critical need to identify those patients with extremely low risks of breast cancer recurrence to spare these patients unnecessary over-treatment of their disease.

The Application of Microarray Technologies to Breast Cancer

RNA expression of individual genes can be detected and quantified by a variety of techniques, such as Northern blot analysis, S1 nuclease protection, differential display, and serial analysis of gene expression or SAGE (Alwine et al., 1977; Berk and Sharp, 1977; Liang et al., 1992; Velculescu et al., 1995). Recently two array-based technologies, cDNA and oligonucleotide arrays, have been applied to gene expression quantification (reviewed in Cooper et al (Cooper, 2001)). Simply defined, a microarray is an orderly arrangement of known and est (expressed sequence tag) DNA sequences attached to a solid support that can be interrogated with cDNA or genomic DNA. The advantage of the newer microarray technologies is the ability to measure the RNA expression of thousands of genes at one time, and to relate how the gene expression pattern of one gene correlates to the expression of other genes in or between different tumor samples, or to measure DNA amplification or loss of DNA. The simplicity of experimental design for microarray analysis provides a vehicle to tackle the complex nature of the breast cancer genome with exquisite detail. However, emerging from early experience with this technology, there is a growing appreciation that “more data” is not necessarily better. Experimental design issues will be the subject of a later section.

Since the RNA expression microarray technology provides a method for monitoring the RNA expression of many thousands of human genes at one time, there was considerable anticipation that it would quickly and easily revolutionize approaches to cancer diagnosis, prognosis, and treatment. The reality remains extremely promising but is also complex. A potential complication in the application of microarray technology to primary human breast tumor samples is the presence of variable numbers of normal cells, such as stroma, blood vessels, and lymphocytes, in the tumor. Indeed, it has been demonstrated using gross analysis of human breast cancer specimens compared with breast cancer cell lines, that the tumors expressed sets of genes in common not only with these cell lines, but also with cells of hematopoietic lineage and stromal origin (Perou, 2000). Laser capture microdissection has also been successfully used to isolate pure cell populations from primary breast cancers for array profiling (Sgroi et al., 1999). In this seminal paper, Sgroi et al. (1999) utilized laser capture microdissection to isolate morphologically “normal” breast epithelial cells, invasive breast cancer cells, and metastatic lymph node cancer cells from one patient, and was able to demonstrate the feasibility of using microdissected samples for array profiling, as well as following potential progression of cancer in this patient. However, with the emerging data supporting important roles for the surrounding stroma in breast cancer progression (reviewed in (Chrenek et al., 2001; Haslam and Woodward, 2001), and the labor-intensive and technically challenging nature of laser capture technology with subsequent amplification of RNA for quantitation, most published investigations to date have evaluated total gene expression to identify prognostic profiles, as will be described below.

Expression Microarray Analyses for the Identification of Prognostic Factors

Many of the first explorations into the use of expression microarrays were designed to evaluate the technology for molecular and/or morphologic phenotyping of breast tumors. One of the first comprehensive attempts to characterize the variation in gene expression between sporadic breast tumor samples was published by Perou et al. (2000). This groundbreaking study was the first to establish that tumors could be phenotypically classified into subtypes distinguished by differences in their expression profiles. Perou et al. examined 40 breast tumors, and 20 matched pairs of samples before and after doxorubicin treatment in their study; tumor samples were grossly dissected. An “intrinsic gene set” of 476 cDNAs were selected that were more variably expressed between the 40 sporadic tumors, than between the paired samples. This intrinsic gene set was then used to cluster and segregate the tumors into four major subgroups: 1) a “luminal cell-like” group expressing the estrogen receptor (ER), 2) a “basal cell-like” group expressing keratins 5 and 17, integrinβ4, and laminin, but lacking ER expression, 3) an Erb-B2-positive subset, and 4) a “normal” epithelial group.

A subsequent study by this group has extended the molecular profiling of breast cancer by applying their intrinsic gene set to cluster 78 cancers (the tumors from their previous study were included in these), 3 fibroadenomas, and 4 normal breast tissue samples (Sorlie et al., 2001). The same subgroups were found as before (Perou et al., 2000), except the luminal, ER-positive group was subdivided into further subsets with distinctive gene expression profiles. Since clinical outcomes were available on some of the patients, the authors also examined whether their phenotypic profiles could function as prognostic factors. Univariate survival analysis was performed on 49 patients from the study with locally, advanced disease, but without evidence of distant metastasis. Although ER-positivity was not a significant prognostic factor on its own in this analysis, the luminal-type group enjoyed a more favorable survival (Sorlie et al., 2001) compared to the other groups. Conversely, the basal-like group had a significantly poorer prognosis. This study is clearly encouraging that significant differences in outcome can be ascertained from microarray expression profiling.

More recently, van't Veer et al. (van't Veer) have used RNA expression microarray analyses to identify a 70 gene prognostic signature (“classifier”) in young, axillary lymph node-negative patients using a training set of 78 tumors, and then tested the classifier in a validation set of 19 tumors. The study used a case/control design and employed 5 years of clinical follow-up to define their “good” (controls) versus “poor” (cases) prognosis patients. The optimally accurate prognostic classifier correctly predicted disease outcome for 65 out of the 78 (83%) patients, identify poor prognosis outcomes with a sensitivity of 85% and good outcomes with a specificity of 81%. Thus, the study demonstrates the feasibility of molecular profiling for sub-classification of patient outcomes using undissected clinical material. However, it is known that more than 40% of recurrences in node-negative women will occur after 5 years, thus bringing into question whether this study is representative of all recurrences.

Van de Vijver et al. (2002) have now extended this study with 234 additional young (<53 years), stage I-II breast cancer patients with both node-negative and node-positive disease using the 70 classifier genes from the earlier study (van't Veer et al., 2002) to classify the patients. The authors were able to classify patient outcomes (sensitivity=93%, specificity=53%) that are consistent, or perhaps better than estimates which can be obtained with current prognostic indices. There are several clinical notes about this study, in addition to the short follow-up of these patients. First, is the young age of the patients. Based on the age structure of the US population in 2000 and the most recent, publicly available age-specific incidence rates from SEER (available on the World Wide Web), at least 79% of women with breast cancer in the US will be 50 years old or older. We also know that there are great differences in biological markers, such as ER status and proliferation rates, between younger and older patients, which may complicate the marker profiles identified in this study. Finally, the confounding effects of treatment need to be considered (Borg et al., 2003). In the good prognosis group, it is impossible to dissociate a less aggressive gene profile from responsiveness to the adjuvant treatment. Thus, these data are problematic in that one cannot tell whether the markers reflect those important for the natural history of the disease without treatment, or are solely related to the treatment received.

Several groups have identified specific gene expression profiles associated with ER-positivity using expression microarray analysis of human breast tumors (Perou et al., 2000; van't Veer et al., 2002) (West et al., 2001; Dressman et al., 2001) or SAGE analyses (Porter et al., 2001). The ER itself, along with other estrogen-induced genes, has been shown to be characteristically expressed in “good” prognosis patient subsets using microarray analysis. This is perhaps not surprising given the important position of the ER signaling pathway in breast cancer progression (Fuqua, 2002), and that ER is expressed in about 75% of women with invasive breast cancer (Harvey, 1999). ER expression is an important prognostic factor predicting the natural history of breast cancer after surgery; ER-positive patients have a longer interval to recurrence, and many studies have shown an improved overall survival as well. ER positivity is also a good predictor of response to hormone therapy in women with invasive breast cancer (reviewed in (Elledge et al., 2000); tamoxifen adjuvant therapy halves the 10-year recurrence risk of patients with ER-positive tumors, and reduces the risk of death from metastatic disease by 26% (Early Breast Cancer Trialists' Collaborative Group, 1998).

An interesting study was reported by Gruvberger et al. (2001) who profiled 58 grossly dissected primary invasive breast tumors, and used artificial neural network analysis to predict the ER status of the tumors based on their gene expression patterns. They then determined which specific genes were the most important for ER classification. By comparing to SAGE data from estradiol-stimulated breast cancer cells, they determined that only a few genes of the many genes that were associated with ER expression in tumors were indeed estrogen-responsive in cell culture. This observation lends further support to the hypothesis developed by Perou et al. (Perou et al., 2000) that basic cell lineages, such as the luminal ER-positive cell type, can be partly explained by observed genomic gene expression patterns, rather than downstream effectors of only one pathway, such as the ER.

A few investigators have begun to study putative precursor lesions of invasive disease, such as ductal carcinoma in situ (DCIS), using genomic approaches. Porter et al. (2001) have exploited SAGE analysis to compare 2 SAGE libraries prepared from DCIS, to 2 libraries each of normal, invasive, and metastatic cancer. Of note is that the authors used either manual macrodissection, or magnetic bead separation specific for epithelial cell content to prepare these libraries. They found that tumors of different histology had very distinct gene expression patterns. However, no genes seemed to be specific only for the DCIS or metastatic lesions. Interestingly, the most profound expression pattern changes were found to occur during the early normal to DCIS transition, suggesting that this type of study might identify future targets for chemoprevention.

Recently, Adeyinka et al. (2002) have performed a systematic study comparing 6 cases of DCIS with necrosis, to 4 cases without necrosis utilizing manual microdissection or laser capture microdissestion to prepare the samples for microarray analysis. These authors report that only 69 genes were consistently and differentially expressed between the two histological types of DCIS lesions. Genes important for angiogenesis were notably increased in the DCIS with necrosis group of tumors, as well as other genes involved in migration and hypoxia. Thus this study demonstrates that although gene expression is mostly similar between morphologically distinct types of neoplasia, differences in expression can be identified using expression array profiling, providing hope that this technology will provide profiles predicting cell behavior in early breast disease. Since it has been demonstrated that very early precursor lesions, such as atypical ductal hyperplasia, are genetically related to invasive cancer, and are indeed precursor lesions (O'Connell et al., 1994; O'Connell et al., 1998), there is much anticipation that these lesions will provide valuable information about the origin and etiology of early disease. However, systematic microarray analyses with ductal hyperplasias have yet to be reported, probably due to their rare inclusion in established frozen tumor banks, and their small size.

A few studies have utilized new genomic approaches for the study of inherited breast cancer (reviewed in (Hedenfalk et al., 2002)). There is accumulating evidence, both epidemiological and histological, that tumors arising as a result of mutations in the two breast cancer susceptibility gene families (BRCA1 and BRCA2) are biologically distinct. For instance, BRCA1 breast cancers are most often ER and PR-negative, but BRCA2 cancers more often tend to be positive for these receptors (Verhoog et al., 1998; Osin et al., 1998). In a seminal paper published by Hedenfalk et al. (2002), 7 tumors each from BRCA1 and BRCA2 gene mutation carriers, or sporadic breast cancers were compared by expression microarray analysis. They found that the gene expression profiles of the three tumor groups differed significantly from each other, underscoring the fundamental differences between BRCA1 and BRCA2 mutation-associated tumors. Of course a potential confounding issue was the differential distribution of ER between the BRCA1 and BRCA2 tumors. However, even after removal of ER/PR-associated genes from the analysis, the two inherited tumor groups were still discernable. Thus, ER status alone does not fully explain the observed differences in gene expression profiles. Although this study is obviously very small, and other confounding issues such as tumor stage, grade, and treatment were not able to be considered, it does set a foundation for larger validation studies to confirm differential genes which could then provide important clues to the etiology of inheritable breast cancer.

Expression Microarray Analysis of Metastatic Breast Cancer Behavior

There is a growing understanding of the basic biology of the metastatic process (reviewed in (Welch et al., 2000)), and cancer metastasis is known to be an inherently inefficient process with only a subset of micrometastases persisting to form clinically evident metastases. Thus, the detection of breast cancer cells in the blood stream, or in secondary organs such as lymph nodes or bone marrow, does not always predict the ability of the primary tumor to form viable distant metastases. In order to increase the survival of breast cancer patients, an increased understanding of the key genes and mechanisms supporting metastatic behavior of human breast cells needs to be elucidated. Although it can be argued that treatment with metastasis-targeting agents may be of limited value, metastasis prevention in the advanced disease setting may have a clinical role by preventing secondary metastases as tumors progress.

Using 9 paired primary and axillary lymph node tumor samples, it has been determined that the gene expression patterns within each pair were more similar than between pairs (Parra et al., 2002), suggesting that gene expression patterns necessary for metastasis are probably already present in the primary lesion. It can be interpreted that these data further validate the use of primary tumors for studying recurrence and metastatic behavior. This result is also consistent with the original “soil and seed” hypothesis of Paget (1989) which suggests that tumor sub-populations with metastatic potential preexist in the primary tumor. These are disseminated, and then encouraged to colonize at other sites depending on the microenvironment in the distant organ.

Unfortunately, distant metastatic tumor samples from breast cancer patients are rarely biopsied or stored in tissue banks, thus these tumors are a very rare resource that have infrequently been examined by microarray analyses. However, the vast majority of human breast cancer cell lines were originally derived from metastatic breast lesions, and thus are a surrogate, albeit incomplete since the stromal tumor component is lacking, of the metastatic phenotype. A number of investigators have used human breast cancer cell lines for microarray analysis. Nacht et al. (1999) were among the first to describe expression differences between primary and metastatic paired cell lines (termed 21PT and 21MT, respectively). Furthermore, they validated this differential gene expression in 7 primary breast tumors, and 10 metastatic tumors using a custom array of genes differentially expressed in the paired cell lines. The expression of several genes that have been profiled in human tumors (Perou et al., 2000) was found to be associated with the metastatic phenotype, including mucin 1, c-Erb-B2, and thrombospondin (Nacht et al., 1999). Schwirzke et al. (2001) have used two sublines of MDA-MB-435 cells, one metastatic and one nonmetastatic, to profile metastasis-related gene expression. They report that the metastatic phenotype, as expected, was associated with deregulation of genes involved in motility, transmemebrane signaling, and extracellular matrix function. Unexpectedly, they also identified genes involved in the immune response to be lower in the metastatic subline, suggesting a mechanism for tumor metastasis “escape” from immune surveillance. A number of other groups have also profiled human breast tumor cell lines with different invasive and metastatic properties (Zajchowski et al., 2001; Ross et al., 2000), and have attempted to correlate these phenotypes with gene expression patterns, but definitive conclusions are incomplete due to the heterogeneity of the cell lines examined. Thus, the approach of using paired sublines with differential metastatic phenotypes appears to be a more optimum approach to identify genes specifically associated with metastatic behavior.

The Use of Tissue Microarrays (TMAs) for Confirming Protein Expression and Gene Alterations in Clinical Samples

New genome-wide techniques, such as expression and CGH arrays, which evaluate thousands of genes in a single experiment, can comprehensively profile RNA and DNA from tumors. A companion array-based, high-throughput technique called TMA (Kononen et al., 1998), and reviewed in (Kallioniemi et al., 2001) has been applied to the problem of confirming expression changes predicted by microarray experiments. TMAs have many advantages when evaluating large numbers of clinical samples. A simple high density TMA may contain up to 1000 cells (0.6 mm core) from donor paraffin blocks representing 200 to 500 cases, depending on how many replicate cells are used. This technology subsequently reduces the number of slides to cut and stain, preserving samples, and increasing efficiency in evaluation. Good agreement between traditional methods and TMA analysis of markers has yielded significant associations with survival, similar to that found by traditional analysis on large tissue sections (Torhorst et al., 2001; Zhang et al., 2003). Thus, it does not appear that tissue heterogeneity severely hinders the use of TMAs for these types of protein-based studies.

In the previously described study by Sorlie et al. (2001), an association between the expression of cytokeratins 17 and/or 5 RNA, and poor clinical outcome was observed. In a follow-up validation study, van de Rijn et al. (2002) used TMAs of over 600 breast tumors to confirm predicted clinical associations, and to demonstrate that protein expression of these cytokeratins was a prognostic factor in node-negative breast cancer, independent of tumor size and nuclear grade. Ginestier et al. (2002) also confirmed one-third of the predicted RNA changes in 55 breast tumors using TMA analysis of 15 marker candidates. Similarly, genomic alterations predicted by CGH microarray analyses of breast tumors, such as the association between cytokeratin 5 and 6 positivity and negative ER status, have been confirmed at the protein levels using TMAs (2002). Finally, fluorescence in situ hybridization to TMAs have been recently employed to survey chromosome 17q23 changes identified using CGH (Andersen et al., 2002). These studies clearly demonstrate the value of TMAs to confirm multiple biomarker expression at the tissue level in clinical samples.

Microarray Analysis to Identify Predictive Biomarkers

A predictive marker is defined as a biological factor which can predict clinical outcome in treated patients. Thus there are two types of questions which need to be addressed. First, who needs treatment? Prognostic factors are useful to identify a “poor prognosis” group who could benefit from treatment. The second question is of those who need treatment, which treatment should they receive? Predictive factors would be useful to answer this later question. Systemic chemotherapy for operable breast cancer significantly decreases the risk of relapse and death (Early Breast Cancer Trialists' Collaborative Group, 1998; Early Breast Cancer Trialists' Collaborative Group, 1991). However, although these large clinical trials have confirmed the value of systemic therapy, it is not possible to identify at the outset those patients who are likely to respond to adjuvant treatment or which type of treatment should be used. Thus, there is a need to identify breast cancer patients who will benefit from specific adjuvant therapies, while sparing others from the side effects of futile treatment. Unlike patients with advanced breast cancer, in whom response can be assessed by tumor measurements after a few cycles of treatment, patients with early breast cancer have no measurable disease after primary surgery. Thus, no methods are now available to separate patients likely to respond to standard adjuvant treatment from those more likely to benefit from alternative therapies. This is because can not yet answer first question, prognosis, adequately. Because of these arguments, the accepted practice is to prescribe adjuvant chemotherapy even if the expected benefit is low (Fisher et al., 1997). A good example of this practice is that give everyone with ER-positive disease tamoxifen therapy, even though know that only 60% will respond to this treatment.

Treatment given before surgery (neoadjuvant therapy) has a number of advantages in breast cancer including earlier assessment of response to therapy, and access to the primary tumor during early treatment for in vivo testing for predictive markers whose expression correlates with successful treatment. Unlike response in the metastatic setting where one can measure response at metastatic sites, but can not estimate effects on survival, response to neoadjuvant chemotherapy is a validated surrogate marker for improved survival and may be used to test the efficacy of treatment regimens. In the NSABP B-18 study, survival outcome was better in patients whose tumors responded to neoadjuvant chemotherapy compared to those who had chemotherapy-resistant disease (Fisher et al., 1998). These data indicate that tumor response to neoadjuvant chemotherapy correlates with outcome, and the response in the primary tumor mirrors the effect of chemotherapy on micrometastases (Fisher et al., 1998). Likewise, in a study involving 158 patients, clinical response to neoadjuvant chemotherapy was found to closely correlate with improved clinical outcome (Chang et al., 2000). By multivariate analysis, good clinical response to neoadjuvant chemotherapy was the only independent variable associated with decreased risk of death (Chang et al., 2000). With neoadjuvant chemotherapy, the primary breast cancer provides a unique opportunity for assessing predictive markers and for studying hypothesis-generating relationships, in that it allows for measurements of possible biologic determinants to be made before treatment in an intact human tumor.

Studies have been conducted assessing the amount of total RNA obtained from each core biopsy of primary breast cancers undergoing neoadjuvant chemotherapy for its use in expression microarray experiments. From each core biopsy, sufficient total RNA was extracted for oligonucleotide array analysis and preliminary patterns predictive of sensitivity and resistance to specific treatments have been reported (Chang et al., 2002), where others report 45% (Buchholz et al., 2002) or as high as 93% (Ellis et al., 2002) of core biopsies to yield sufficient high quality RNA for array analysis. Other investigators have reported faithful linear RNA amplification protocols using limiting amounts of RNA from microdissected breast tissues (Aoyagi et al., 2003; Zhao et al., 2002). Further work is essential in integrating amplification protocols into large-scale microarray analysis, and validating these pilot predictive expression patterns in independent patient cohorts.

A neoadjuvant approach was also undertaken by Buchholz et al. (2002) to look at the effects of chemotherapy on gene expression. The authors obtained sufficient RNA from core biopsies of 5 patients to obtain serial microarray expression profiles. Patients with good pathological responses to neoadjuvant treatment had gene profiles that clustered distinctly from those of patients who were poor responders to treatment. Unfortunately, all the patients had different gene expression changes after chemotherapy, with no single gene expression changes significantly associated with response in all 5 patients. Their result could be due in part to the small number of patients examined, and the heterogeneity of treatments in this study. However, combined neoadjuvant treatment approaches, and expression microarray technology offers a potentially clinically useful method for developing predictive tests for chemotherapy sensitivity that when validated, may reduce unnecessary treatment for women with breast cancer.

Experimental Design and Statistical Analysis

As seen above, genomic approaches can address a wide range of objectives important in breast cancer. These include, for example, molecular subclassification of breast cancer, characterization of pathways important in breast cancer etiology and progression of premalignant lesions, and prognostication of natural history or prediction of benefit to specific therapies. The first two studies focus on discovering new classes of samples or genes, while the latter two are examples of problems in classification.

At best genomic experiments can generate a gold mine of data that may, with proper “mining,” help shed light on questions far beyond those originally envisioned. At worst, without careful planning these expensive and complex experiments may fail to illuminate even their primary objectives. In all cases it is very important to minimize possible sources of confounding factors. Samples should be handled and prepared in as identical a manner as possible. Standard methods, such as blinding of samples to the laboratory staff, and processing of the samples in batches that include examples of all relevant classes, is common practice in single gene studies and is even more important here.

In clinical trials, sample sizes are planned ahead of time to ensure that the number of subjects to be enrolled will be adequate to address the question. Reporting guidelines now include planned sample sizes and target effect sizes. Traditional prognostic or predictive studies are beginning to follow suit. In sharp contrast, sample sizes in most genomic (expression arrays, CGH, SAGE) experiments to date appear to have been determined by the limited number of frozen samples available and the cost of arrays. As a result, studies have tended to be very small. In the future, as studies are undertaken that propose to change clinical practice, larger samples sizes, that are more likely to encompass the full diversity of the target population, will be required. Thus, reviews for funding of such studies are beginning to require more rigorous justification.

Study objectives also determine the most appropriate methods of analysis. To date, class discovery studies have used unsupervised methods, especially cluster analysis, to “discover” sample or gene groupings. Such studies are generally exploratory or hypothesis-generating and confirmation of results often relies on subsequent correlation with further supplemental biological or bioinformatic data. Analysis generally proceeds in steps, beginning with filtering of genes and samples to remove poor quality samples, and uninformative or poorly measured genes. This is followed by clustering or data mining designed to uncover “hidden” groups or relationships. The “significance” of such groups or relationships can be difficult to assess because any dataset, even a randomly generated one, can be clustered. Fortunately, methods have been proposed to assess the stability or reliability of the clustering that may help distinguish real from spurious results (Dudoit et al., 2002; McShane et al., 2002). To the knowledge of the inventors, there are no standard methods to determine an appropriate sample size for such studies.

Class prediction has typically been addressed with a case/control type of design (i.e. ER-positive vs. ER-negative; disease free vs. relapsed), and samples are included because of their known status. All other things being equal, the most powerful discrimination of groups is obtained when cases and controls are equally represented. Cluster analysis has sometimes been used in the analysis of such studies in the hope that groups will cluster together, but, as pointed out by Simon et al (Simon et al., 2003), unsupervised cluster analysis is not effective for class comparison or class prediction. When the goal is discrimination or the selection of features that discriminate, the analysis should make use of the available information. As with class discovery, analysis begins with filtering of genes and samples to remove poor quality samples and unexpressed or poorly measured genes. Analysis then proceeds to select a subset of “informative” genes, compute a score or index, and finally to define a classification rule. The process is often iterative, and the score may be a simple weighted average of gene expression, as in linear discriminant analysis, or a complicated non-linear function, as in artificial neural networks. However the classifier is computed and the classification rule defined, it is of little value if it cannot be shown to generalize to other samples. Performance is usually assessed by the misclassification error rate, and by summary statistics borrowed from the field of diagnostic testing, such as sensitivity, specificity, and false positive rate. “Resubstitution” estimates of classification success can be computed by classifying the same cases used to create the classifier, but the estimates are biased and often highly overly optimistic. The potential for overfitting, a well-known problem even in traditional single gene prognostic and predictor factor studies (Hilsenbeck et al., 1992; Hilsenbeck and Clark, 1996; Altman et al., 1994), is simply made worse by the huge number of explanatory variables and small sample sizes.

Classifier performance is best tested by applying it to a completely new, independent set of samples. Despite some methodologic problems, the studies of van't Veer et al and van de Vijver (2002; 2002) are ground-breaking examples. The external validation set should include all of the types of cases in the training set, and the assay process should be replicated as closely as possible. Gruvberger et al. (2002) have suggested that cases in validation sets should be carefully matched on known prognostic markers such as ERα, in part, because they were unable to discriminate good and poor outcomes in their own set of tamoxifen-treated, node-negative cases using genes from the van't Veer study (2002). This hardly seems a fair comparison—the van't Veer cases were untreated, and gene expression was not measured with the same array or reference RNA's (Gruvberger et al., 2001). It should be kept in mind that lack of assay consistency has plagued the interpretation of studies of the prognostic and predictive values of Her-2/neu and immunohistochemical assessment of hormone receptors for years. In addition, matching may cause more problems than it solves because factors used to match cannot be evaluated for their effect.

When fully independent external validation is not possible, then some other method, such a cross-validation, must be used to obtain unbiased estimates of classifier performance. Properly implemented, leave-one-out cross validation and related methods can provide nearly unbiased estimates of classifier performance. In order for the estimates to be reliable, however, it is absolutely critical that the cross-validation be external to the entire process by which the classifier is created (Simon et al., 2003; Ambroise and McLachlan, 2002). That is, in leave-one-out cross-validation, one sample is selected to left out. The entire analysis including normalization, expression estimation, filtering, gene selection, weighting, and classifier rule construction is performed on the remaining samples. The left-out sample is then processed and classified. The process is repeated leaving out and then classifying each sample in turn. Since each left-out case will be classified by a slightly different classifier, the resulting classification error is a nearly unbiased estimate of the classification error rate of the classifier construction process, not the error rate of a specific classifier. A final classifier is usually constructed by the same process, using all the data. Of course, independent validation is still important, especially if the training sample is relatively small because any estimates of accuracy will have wide confidence intervals. For example, in a study of fifty or fewer samples, a cross-validated error rate of 15% will have a 95% confidence interval of 6 to 27%, a range far too wide to guarantee good performance on future samples. While the entire multivariable classification problem is too complex for useful sample size calculations, simpler approaches can be useful. These can be based on detecting modest differences in individual genes (gene selection phase) with good power (i.e. 80-90%) at a stringent level of significance (i.e. 0.1 to 1%) that will help control for multiple comparisons. Sample size should also take into account the desired width of confidence intervals for the cross-validated or independent validation error rates.

### SUMMARY

It is the goal of comprehensive, genomic-wide approaches to identify clinically useful genetic profiles that will accurately identify diagnostic subtypes, and predict prognosis and treatment responsiveness of breast cancer patients. Clearly, the management of patients would be optimized if clinicians had a molecular profile of a patient's tumor at the time of diagnosis that would accurately identify those patients who could be spared unnecessary treatment of their disease, or alternatively whose prognosis was so poor that aggressive therapies are warranted and to pinpoint the optimal therapy. It is obvious that single gene studies have to be replaced with the newer molecular approaches of microarray analysis. Undoubtedly, the benchmark for any newly identified biomarker or biomarker DNA or RNA expression profile arising from these new microarray technologies will have to be its comparison to standard prognostic factors.

The importance of experimental design to ask the appropriate question in the available data set can not be overly stressed. Similarly, validation of generated profiles must be performed in independent data sets. The lessons learned in years of prognostic and predictive factor identification and implication need to be implemented in microarray approaches for the management of breast cancer. Obviously it is hoped that this new technology will greatly improve the ability to diagnose, and predict the outcomes of breast cancer patients. The following references generally regard the art for breast cancer prognosis.

U.S. Patent Application Publication US 2003/0198972, U.S. Patent Application Publication US 2004/0002067, U.S. Patent Application Publication US 2003/0186248, WO 03/060164, WO 03/083141, and WO 03/060470 regard breast cancer progression signatures. In particular, the signatures comprise expression of more than one gene, particularly embodied on a microarray. In specific embodiments, particular genes listed therein are present on the array.

U.S. Patent Application Publication US 2004/0058340, U.S. Patent Application Publication US 2003/0224374, and WO 02/103320 concern genetic markers for breast cancer, particularly to provide information on tumor metastasis and also particularly for detecting the presence or absence of genes identified therein, such as the estrogen receptor ESR1 and BRCA1. Other methods encompass classifying cell samples as ER(+) or ER(−).

U.S. Patent Application Publication US 2003/0236632 describes methods of determining the presence of abnormal breast cells in human subjects by identifying the increased expression of CRIP 1 or HN 1 sequences.

In light of the above, the present invention provides a novel and long-felt need for predicting resistance to a chemotherapy utilizing RNA expression profiling.

### BRIEF SUMMARY OF THE INVENTION

Breast cancer is the most common malignancy afflicting women from Western cultures. It has been estimated that approximately 211,000 women will be diagnosed with breast cancer in 2003 in the United States alone, and distressingly each year over 40,000 women will die of this disease. Developments in breast cancer molecular and cellular biology research have brought us closer to understanding the genetic basis of this disease. Unfortunately, this information has not yet to be incorporated into the routine diagnosis and treatment of breast cancer in the clinic.

Recent advances in microarray technology hold the promise of further increasing understanding of the complexity and heterogeneity of this disease, and providing new avenues for the prognostication and prediction of breast cancer outcomes. However, many of these methods compare resistant cells vs. sensitive cells in cell lines, which may be inappropriately extrapolated to molecularly distinctive in vivo tumors. Furthermore, the experimental design related to the resistant cells in known methods provides molecularly inapplicable information. The present invention employs microarray technology in an advantageous and novel manner for predictions related to breast cancer therapy.

In one embodiment of the present invention, there is a method of predicting the response of an individual to a chemotherapy, comprising the steps of providing the level of one or more expressed RNAs from an individual having tumors sensitive to the chemotherapy; providing the level of at least some of the one or more expressed RNAs from a different individual on the chemotherapy, said level from tumors that have occurred during the chemotherapy and/or tumors present in the individual before the chemotherapy but that become resistant during the chemotherapy; and comparing the level of at least one commonly expressed RNA between the individuals. In a specific embodiment, the level of one or more expressed RNAs from the tumors that have occurred during the chemotherapy or that become resistant during the chemotherapy is higher than the level of the one or more expressed RNAs in the tumors sensitive to the chemotherapy.

In some embodiments of the invention, when the level of the one or more expressed RNAs is higher in the tumors that have occurred during the chemotherapy or that become resistant during the chemotherapy, the tumors of the individual have become resistant to the chemotherapy. In a specific embodiment, the levels of one or more RNAs from an individual sensitive to the chemotherapy is provided at least in part from a known standard.

In a specific embodiment, the level of one or more expressed RNAs from the tumors that have occurred during the chemotherapy or that become resistant during the chemotherapy is lower than the level of the one or more expressed RNAs in the tumors sensitive to the chemotherapy. In some embodiments of the invention, when the level of the one or more expressed RNAs is lower in the tumors that have occurred during the chemotherapy or that become resistant during the chemotherapy, the tumors of the individual have become resistant to the chemotherapy. In a specific embodiment, the levels of one or more RNAs from an individual sensitive to the chemotherapy is provided at least in part from a known standard, for example.

In a specific embodiment, providing the level of RNAs from tumors that have occurred during the chemotherapy or that become resistant during the chemotherapy comprises the following steps: obtaining one or more cells from tumors that have occurred during the chemotherapy or that become resistant during the chemotherapy; isolating RNA from the one or more cells; and determining the level of one or more of the RNAs. In further specific embodiments, the RNA levels are determined by microarray analysis. In some aspects of the invention, the tumors that occurred during the chemotherapy occurred within about one month to about one year from initiation of the chemotherapy. In particular embodiment, the individual has breast cancer. Furthermore, the chemotherapy may be a hormone therapy, such as one that comprises tamoxifen.

In specific embodiments of the invention, the RNAs are expressed from one or more polynucleotides listed herein in Table 1, Table 2, or both. In specific embodiments, one of the expressed RNAs is DUSP6. In additional specific embodiments, when the method predicts the cancer as resistant to the chemotherapy, the individual is subjected to an alternative cancer treatment, such as one comprising chemotherapy, radiation, surgery, gene therapy, immunotherapy, hormone therapy, or a combination thereof. In additional specific embodiments, the alternative cancer treatment is chemotherapy and comprises raloxifene, ZD1839, trastuzumab, letrozole, or a combination thereof.

In another embodiment of the invention, there is as a composition of matter expressed RNAs the levels of which are indicative of resistance to a chemotherapy, wherein one or more of the expressed RNAs are listed herein in Table 1, Table 2, or both. In specific embodiments, the expressed RNAs are comprised on a substrate, such as a microarray chip. In specific embodiments, the chemotherapy comprises tamoxifen.

In an additional embodiment of the present invention, there is as a composition of matter an RNA expression profile comprising DUSP6. In particular embodiments, the level of DUSP6 is indicative of resistance to tamoxifen.

In another embodiment of the present invention, there is a method of determining resistance to a chemotherapy in the cancer of an individual, comprising the step of identifying the expression level of DUSP6 in one or more cancer cells in the individual. In a specific embodiment, the chemotherapy comprises tamoxifen. In specific embodiments, the method is further defined as comparing the level of DUSP6 in one or more cancer cells of the individual with the level of DUSP6 from one or more cells that are sensitive to the chemotherapy. In additional embodiments, when the level in the one or more cancer cells of the individual is higher than the level in one or more cells that are sensitive to the chemotherapy, a cancer of the individual is resistant to the chemotherapy. The identifying step may comprise identifying an expressed DUSP6 RNA level, and the identifying step may be further defined as comprising microarray analysis. In particular embodiments, when the cancer is resistant to the chemotherapy the method further comprises subjecting the individual to an alternative cancer treatment, such as chemotherapy, radiation, surgery, immunotherapy, hormone therapy, gene therapy, or a combination thereof.

In another embodiment of the invention, there is a method of predicting the response of an individual to a chemotherapy, comprising the steps of: providing the level of one or more expressed polynucleotides from an individual on chemotherapy, said level from tumors that grow during or after the chemotherapy; and comparing the level of one or more expressed polynucleotides to a control, wherein a difference between the level of at least one expressed polynucleotide predicts resistance to chemotherapy in the individual. In a specific embodiment, the difference between the levels is defined as being higher in the individual than the control, as being lower in the individual than the control, or a combination of expressed polynucleotides being higher or lower in the individual compared to the control. In another specific embodiment, the difference between the level of at least one expressed polynucleotide in the individual and the control is greater than about one-fold.

In particular embodiments, the individual in which resistance is predicted for is a human, although the invention is suitable for any mammal, including dogs, cats, horses, and so forth. The individual may have any cancer capable of becoming resistant to a chemotherapy, including breast cancer, lung cancer, prostate cancer, pancreatic cancer, brain cancer, skin cancer, ovarian cancer, cervical cancer, testicular cancer, liver cancer, spleen cancer, kidney cancer, colon cancer, and so forth. In preferred embodiments, the cancer is breast cancer. In further specific embodiments, the cancer is a solid tumor.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 illustrates clustering of representative genes in RNA expression profiling methods of the present invention (p=0.01).

FIG. 2 illustrates clustering of representative genes in RNA expression profiling methods of the present invention (p=0.05).

FIG. 3 demonstrates statistical differences between treatment with Tam in vector control and MKP3 overexpressing cells were determined using Student's t-test. Box and whisker plots of MKP3 RNA expression in 9 tumors. The values for minimum, maxiumum, and 25th and 75th percentiles of expression are indicated.

FIGS. 4A-4C demonstrate studies of overexpression of MKP3 related to tamoxifen resistance. In FIG. 4A, there is an immunoblot of an epitope-tagged MKP3 vector. In FIG. 4B, there is an anchorage-independent colony formation assay with MCF-7 cells harboring the MKP3 vector. In FIG. 4C, tamoxifen resistance is demonstrated in athymic mice bearing MKP3-transfected, overexpressing tumors. Estimated average log-transformed tumor size is presented as a function of time in days. Estrogen (n=6 for vector and MPK3). Tamoxifen (n=4 for vector and n=5 for MKP3).

FIGS. 5A-5H show cross-talk between MKP3, MAPK, and Erα signaling pathways. In FIG. 5A, the effect of estrogen or tamoxifen was assessed on the activation of MAPK and ERα. FIGS. 5B and 5C provide quantitation of results provided in FIG. 5A. FIG. 5D shows MKP3 enzymatic phosphastase activity in MKP3-overexpressing breast cancer cells. In FIG. 5E, there is an immunoblot analysis of MKP3 V1 and MKP3-2 transfectants treated with vehicle, E2, or Tam for 2 hours in the absence(−) or presence of PD98059. Immunoblots were stained with antibodies to V5, phospho-pMAPK and S118 ERα, or total MAPK and ERα. In FIG. 5F, an immunoblot analysis of MKP3 V1 and MKP3-2 transfectants treated with vehicle, E2, or Tam for 2 hours in the absence(−) or presence of PD98059 is provided. Immunoblots were stained with antibodies to MKP1, phosphoJNK, total JNK, p38, and total p38. In FIG. 5G, there is a phosphatase assay using pNPP as a substrate using extracts prepared from MKP3 vector 1 and MKP3-2 cells treated for 2 hours with vehicle, E2, or Tam. The nonenzymatic hydrolysis of the substrate was corrected by measuring the control vector transfected immunoprecipitates, and the MKP3 levels were corrected for this level. Phosphatase assays were performed in triplicate, n=3 separate experiments shown. In FIG. 5H, an MKP3/MAPK binding assay was performed with MKP3 V1 and MKP3-2 transfectants treated for 2 hours with ethanol vehicle (C), E2 (E), or Tam (T). Pre- and Post-V5 immunoprecipitated extracts (Pre-IP and Post-IP) were immunoblotted with antibodies to ERK2 and V5 to demonstrated levels of MAPK and MKP3, arrows respectively. Immunoglobulin heavy chain (HC) and light chain (LC) are shown.

FIG. 6 shows a comparison of NR Motifs between ERα coactivators and MKP3.

FIG. 7 studies affects of MKP3 on modulation of ERα activity using transactivation assays with estrogen-responsive luciferase reporter.

FIG. 8 demonstrates affect of MKP3 to modulate the activity of a variety of nuclear receptors using transactivation assays with estrogen-responsive reporters.

FIG. 9 shows whether MKP3 phosphatase activity is not essential for ERα activity.

FIG. 10 demonstrates whether MKP3 was a general transcriptional activator in MCF-7 cells.

FIGS. 11A-11D show immunoblot analysis for MKP3 embodiments. In FIG. 11A, there is immunoblot analysis of two vector control (Con 1 and 2), and two MKP3-overexpressing transfectants (MKP3-1 and 2) treated for 2 hours with ethanol vehicle (C), E2 (E), or Tam (T). Immunoblots were stained with antibodies to CCND1, PR-A and β forms, AIB, and anti-Rho GDI as a loading control. In FIG. 11B, there is a densitometric scan of the immunoblot in panel A showing levels of CCND1 normalized to Rho GDI levels. In FIG. 11C, there is an immunoblot analysis of MKP3 V1 and MKP3-2 transfectants treated with vehicle, E2, or Tam for 2 hours in the absence(−) or presence of PD98059. Immunoblots were stained with antibodies to CCND1, PR, and ERK2 as a loading control. In FIG. 11D, PR ubiquitin assay was performed in MCF-7 cells transiently transfected with expression vectors for ubiquitin (pcDNA-HA-ubiquitin), MKP3-V5, and PR-B (pcDNA 3.1-PR-B), and treated for 2 hours with ethanol vehicle (C), E2 (E), or Tarn (T). The upper panels are immunoprecipitates resolved by SDS-PAGE, and immunoblotted with anti-HA antibody. The same blot was then stripped and reimmunoblotted with anti-PR antibody to demonstrate the levels of introduced PR-B in the lower panel.

FIG. 12 illustrates identification of altered gene expression associated with exemplary tamoxifen resistance in breast tumors.

FIG. 13 demonstrates comparison of EBP50 RNA levels in tamoxifen-sensitive and tamoxifen-resistant breast tumors.

FIG. 14 shows that overexpression of MTA2 in T47D cells is associated with hormone-independent and tamoxifen-resistant growth in soft agar.

FIG. 15 shows decreased expression of EBP50 in MTA2 overexpressing T47D cells.

FIGS. 16A and 16B show that EBP50 binds to HER2.

FIGS. 17A and 17B demonstrates that EBP50 overexpression enhances ERα activity.

FIG. 18 illustrates an exemplary model for a role for EBP50 in tamoxifen resistance.

FIG. 19 shows that RhoGDI represses exogenous ERα activity in Hela cells.

FIG. 20 demonstrates that RhoGDI represses endogenous ERα activity in MCF-7 breast cancer cells.

FIG. 21 shows that RhoGDI decreases the acetylation of ERα level in vivo.

FIG. 22 shows that RhoGDIα is an in vitro substrate of p300 HAT activity.

FIG. 23 demonstrates that RhoGDI exhibits no intrinsic HAT activity.

FIG. 24 shows that RhoGDI is acetylated in vivo.

FIG. 25 demonstrates that the N-terminal region of RhoGDI comprises acetylation site(s).

FIG. 26 shows that RhoGDI decreases ERα access to p300 HAT.

FIG. 27 shows that RhoGDI inhibits p300 acetylation of ERα in vitro.

FIG. 28 demonstrates RhoGDI association with ER in vivo.

FIG. 29 demonstrates that RhoGDI does not bind to ER directly.

FIG. 30 provides an exemplary model for a role of RhoGDI associated with ER.

FIGS. 31A and 31B show that RhoGDI confers resistance to tamoxifen.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The term “expressed RNAs” as used herein refers to RNAs that are transcribed from a polynucleotide. In specific embodiments, the polynucleotide is a gene, such as a gene on a chromosome or mitochondrial DNA. In further embodiments, the expressed RNAs may be isolated from one or more cancer cells, such as one or more cancer cells suspected of being resistant to a hormonal therapy or that are known to be resistant to a hormone therapy. In specific embodiments, the level of the expressed RNA may be determined by determining the level of the RNA molecule or by determining the level of a polypeptide translated from the expressed RNA, such as determining the level by immunoblot, for example.

The term “microarray” as used herein refers to a collection of expressed RNAs, in particular comprised on a substrate, such as a microchip.

The terms “overexpress,” “overexpressed,” or overexpressing” as used herein refers to the level of expression of an RNA being greater than one fold higher compared to a control sample, for example.

The term “predicting” as used herein refers to identifying a chance of developing or having resistance to a chemotherapy.

The term “resistance” as used herein refers to when a tumor starts growing during or after treatment.

The term “RNA expression profiling” or “RNA expression profile” as used herein refers to collecting information from a plurality of expressed genes in the form of RNA transcripts, or the collection thereof, respectively. In alternative embodiments, the gene product of the RNA is assayed for information. In specific embodiments, the plurality of RNA transcripts provides information related to breast cancer therapy. In additional specific embodiments, the information gleaned from profiling facilitates determination of a breast cancer therapy, such as whether or not to employ a particular therapy, for example a hormone therapy. In further embodiments, the hormone therapy is related to estrogen, such as the therapy being an estrogen inhibitor, for example tamoxifen. In particular embodiments, the collection of expressed genes is compared between two samples, and in specific embodiments those samples are from one or more individuals having tamoxifen sensitive tumors and a sample from an individual having metastatic tumors occurring during a particular chemotherapy treatment. In specific embodiments, the comparison provides information whether or not a particular chemotherapy treatment should be utilized or continued for the individual.

The term “tamoxifen-resistant” as used herein refers to a tumor, including the individual cells therein, that is or becomes refractory to treatment by tamoxifen. In specific embodiments, the tamoxifen-resistant tumor becomes resistant to tamoxifen treatment after initiation of the treatment and may occur during the treatment. In further specific embodiments, the resistance to tamoxifen manifests at about 2-24 months while the patient is taking hormonal therapy. In de novo resistance, the patient does not respond to initial therapy. Acquired resistance is where the patient develops metastatic disease during therapy. In additional specific embodiments, resistance to tamoxifen is the result of one or more genes being overexpressed and/or underexpressed, compared to non-cancerous cells of the same tissue.

The term “tamoxifen-sensitive” as used herein refers to a tumor, including the individual cells therein, that is treatable with tamoxifen. In specific embodiments, the tamoxifen-sensitive tumor remains sensitive during the treatment. In further specific embodiments, the tamoxifen-sensitive tumor is still sensitive up to at least about seven to ten years.

The terms “underexpress,” “underexpressed,” or underexpressing” as used herein refers to the level of expression of an RNA being less than one fold higher compared to a control sample, for example.

II. The Present Invention

The present invention concerns the prediction of response to cancer therapy, such as hormone therapy in breast cancer patients, for example, using RNA expression profiling. In particular, information obtained from the present invention will assist a health care provider in determining whether or not a tumor (including cells therein) will become resistant to the hormone therapy or are already resistant to the hormone therapy. In specific embodiments, the present invention will provide direction whether or not to continue with hormone therapy, such as to add or change the cancer therapy. In particular aspects of the invention, tamoxifen is the exemplary embodiment described for illustrative purposes only, and a skilled artisan recognizes that the invention can be utilized for other chemotherapeutic drugs also, including other hormone therapy drugs.

Acquired resistance to hormone therapy such as tamoxifen is well-known in the art. In particular, breast cancer patients while undergoing treatment with tamoxifen have recurrence of the disease. In specific embodiments, the disease metastasizes during therapy with tamoxifen, thereafter to be considered a resistant metastases. The present invention predicts the occurrence of metastases, provides information concerning present metastases and/or prevents additional mestastases by identifying tumors susceptible to becoming resistant or being resistant. In other words, the present invention identifies novel genes that cause or impact on resistance, thereby providing information for specific therapies that are developed for these genes. Furthermore, the genes identified by methods of the present invention are useful as biomarkers to avoid hormonal therapies, such as antiestrogens, including tamoxifen, raloxifene (Evista), and/or fulvestrant, for example, if the likelihood of developing resistance exists.

In particular, the present invention employs an RNA expression profile (which may also be referred to as a gene expression profile), to predict a response to a drug therapy, such as tamoxifen, in breast cancer patients. In specific embodiments, the present invention encompasses molecular events related to resistance, which may also be referred to as adaptive resistance. In particular, the polynucleotides may already be expressed, but their levels change in response to therapy. In specific embodiments, the levels of the polynucleotides become changed or altered when resistance develops.

In the art, many researchers employ microarray experiments designed to compare biologically disparate embodiments by evaluating primary tumors after cessation of tamoxifen resistance in comparison to tamoxifen-sensitive tumors. Specifically, gene expression in tamoxifen-sensitive tumors (such as those which have not recurred at about 10 years following initial diagnosis) is compared to gene expression in tamoxifen-resistant tumors (such as those recurring during treatment).

Alternatively, and advantageously, the present invention employs RNA expression profiling to compare biologically appropriate embodiments by evaluating metastatic tumors during tamoxifen treatment. Specifically, gene expression in tamoxifen-sensitive tumors (such as those that have not recurred at about 10 years following initial diagnosis) is compared to gene expression in tamoxifen-resistant tumors (such as those that recur in less than 2 years during tamoxifen treatment) still being treated with tamoxifen. The invention exploits the biological characteristic of metastatic tumors being molecularly different than the primary tumors because the metastatic tumor has been treated chemically, such as with a drug, for example tamoxifen. The metastatic tumors are molecularly different compared to primary tumors and/or tumors never exposed to tamoxifen because different gene expression patterns manifest as a result of the tamoxifen exposure. Thus, the microarray of the present invention is directed to the metastatic tumors that arose while the patients were on the drug tamoxifen. In specific embodiments, when tamoxifen is used in advanced disease resistance will eventually arise in all tumors, even if they were initially sensitive, and the present invention is useful therein.

In particular embodiments, the individual in which resistance is predicted for is a human, although the invention is suitable for any mammal, including dogs, cats, horses, and so forth. The individual may have any cancer capable of becoming resistant to a chemotherapy, including breast cancer, lung cancer, prostate cancer, pancreatic cancer, brain cancer, skin cancer, ovarian cancer, cervical cancer, testicular cancer, liver cancer, spleen cancer, kidney cancer, colon cancer, and so forth. In preferred embodiments, the cancer is breast cancer. In further specific embodiments, the cancer is a solid tumor.

In the present invention, MKP3 (also referred to as DUSP6) was found to be expressed in Tam-resistant metastatic breast tumors using expression microarray and qRT/PCR analysis. The present inventors have studied the effects of MKP3 overexpression on the development of TR in ERα-positive MCF-7 human breast cancer cells. The present inventors also examined the molecular cross-talk between MAPK and ERα in TR, and investigated MKP3's effects on downstream targets of ERα signaling. Finally, one embodiment of the present invention comprises a unique feedback loop between MKP3 and ERK 1,2 MAPK that was generated by Tam treatment of MKP3-overexpressing cells. In specific embodiments, an “off-off” mechanism of TR involving the disengagement of the MKP3 negative feedback loop results in sustained MAPK activation in the presence of Tam. Thus, in specific embodiments of the invention, MKP3 impacts on ERα and MAPK function in breast cancer cells, and in additional embodiments a resultant feedback loop impacts on the generation of TR.

In additional embodiments of the invention, the levels of the polynucleotides EBP50 and RhoGDIa were indicative of development of resistance to breast cancer therapy.

III. RNA Expression Profiling and RNA Expression Profiles

The RNAs that are indicative of the corresponding predictive response to a chemotherapeutic, such as tamoxifen, in exemplary embodiments, may be any expressed RNA or RNAs that assist in the evaluation of the response of a breast cancer patient to the chemotherapeutic, including tamoxifen, for example. The expression profile may indicate those tumors that are or will become sensitive to tamoxifen.

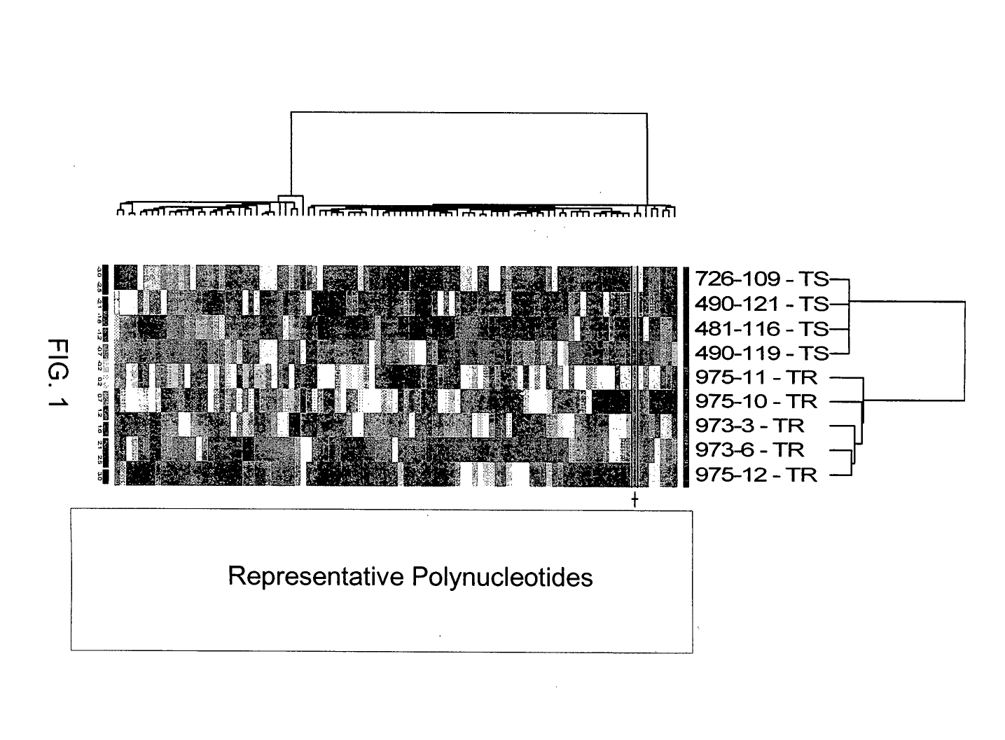

In specific embodiments using methods described herein, FIG. 1 shows a supervised cluster of array data at p>0.01 level of significance and table of genes at this p value (Table 1 with n=98 genes) for an exemplary RNA expression profiling study related to tamoxifen resistance. Table 1 lists the expressed genes in no particular order, and exemplary sequences are provided in the accompanying sequence listing. The first column comprises proprietary numbers. The column concerning “Fold difference of geom means” refers to the geometric mean in the TS group being compared to the geometric mean in the TR group by dividing the TR mean by the TS mean=the fold difference. The Probe set refers to the number given by Affymetrix (Santa Clara, Calif.) for each polynucleotide; the identifier of the sequence is the probe set.

TABLE 1
Genes Identified by Tamoxifen Resistance Expression Analysis (p = 0.01)

| | Fold | | | |
|---|---|---|---|---|
| | difference | | | |
| | of geom | | | |
| | means | Probe set | Description | Gene symbol |
| 65 | 3.178 | 1916_s_at | v-fos FBJ murine | FOS (SEQ ID NO: 1) |
| | | | osteosarcoma viral | |
| | | | oncogene homolog | |
| 88 | 2.874 | 38317_at | transcription elongation | TCEAL1 (SEQ ID NO: 2) |
| | | | factor A (SII)-like 1 | |
| 36 | 2.846 | 39969_at | histone 1, H4c | HIST1H4C (SEQ ID NO: 3) |
| 10 | 2.345 | 37221_at | protein kinase, cAMP- | PRKAR2B (SEQ ID NO: 4) |
| | | | dependent, regulatory, | |
| | | | type II, beta | |
| 71 | 2.29 | 41193_at | dual specificity | DUSP6 (SEQ ID NO: 5) |
| | | | phosphatase 6 | |
| 4 | 2.182 | 39072_at | MAX interactor 1 | MXI1 (SEQ ID NO: 6) |
| 12 | 2.106 | 31792_at | annexin A3 | ANXA3 (SEQ ID NO: 7) |
| 23 | 2.103 | 1577_at | androgen receptor | AR (SEQ ID NO: 8) |
| | | | (dihydrotestosterone | |
| | | | receptor; testicular | |
| | | | feminization; spinal and | |
| | | | bulbar muscular atrophy; | |
| | | | Kennedy disease) | |
| 24 | 2.057 | 39032_at | transforming growth factor | TGFB1I4 (SEQ ID NO: 9) |
| | | | beta 1 induced transcript 4 | |
| 96 | 2.04 | 654_at | MAX interactor 1 | MXI1 (SEQ ID NO: 6) |
| 26 | 1.897 | 1237_at | immediate early response 3 | IER3 (SEQ ID NO: 10) |
| 87 | 1.836 | 38375_at | esterase | ESD (SEQ ID NO: 11) |
| | | | D/formylglutathione | |
| | | | hydrolase | |
| 48 | 1.788 | 35705_at | nuclear receptor subfamily | NR1D2 (SEQ ID NO: 12) |
| | | | 1, group D, member 2 | |
| 40 | 1.703 | 33436_at | SRY (sex determining | SOX9 (SEQ ID NO: 13) |
| | | | region Y)-box 9 | |
| | | | (campomelic dysplasia, | |
| | | | autosomal sex-reversal) | |
| 52 | 1.688 | 1909_at | B-cell CLL/lymphoma 2 | BCL2 (SEQ ID NO: 14) |
| 21 | 1.687 | 41690_at | AT rich interactive domain | ARID5B (SEQ ID NO: 15) |
| | | | 5B (MRF1-like) | |
| 57 | 1.684 | 41141_at | protein-kinase, interferon- | PRKRIR (SEQ ID NO: 16) |
| | | | inducible double stranded | |
| | | | RNA dependent inhibitor, | |
| | | | repressor of (P58 | |
| | | | repressor) | |
| 68 | 1.672 | 31800_at | MRNA; cDNA | |
| | | | DKFZp586L141 (from | |
| | | | clone DKFZp586L141) | |
| 25 | 1.666 | 40838_at | zinc finger protein 292 | ZNF292 (SEQ ID NO: 17) |
| 79 | 1.655 | 36097_at | immediate early response 2 | IER2 (SEQ ID NO: 18) |
| 51 | 1.587 | 34819_at | CD164 antigen, | CD164 (SEQ ID NO: 19) |
| | | | sialomucin | |
| 66 | 1.579 | 38764_at | Dicer1, Dcr-1 homolog | DICER1 (SEQ ID NO: 20) |
| | | | (Drosophila) | |
| 83 | 1.572 | 37294_at | B-cell translocation gene | BTG1 (SEQ ID NO: 21) |
| | | | 1, anti-proliferative | |
| 59 | 1.566 | 35748_at | eukaryotic translation | EEF1B2 (SEQ ID NO: 22) |
| | | | elongation factor 1 beta 2 | |
| 29 | 1.559 | 37661_at | ATPase, Ca++ | ATP2B1 (SEQ ID NO: 23) |
| | | | transporting, plasma | |
| | | | membrane 1 | |
| 31 | 1.542 | 39028_at | karyopherin (importin) | KPNB3 (SEQ ID NO: 24) |
| | | | beta 3 | |
| 2 | 1.541 | 38049_g_at | RNA binding protein with | RBPMS (SEQ ID NO: 25) |
| | | | multiple splicing | |
| 28 | 1.54 | 38765_at | Dicer1, Dcr-1 homolog | DICER1 (SEQ ID NO: 20) |
| | | | (Drosophila) | |
| 15 | 1.531 | 38242_at | B-cell linker | BLNK (SEQ ID NO: 26) |
| 61 | 1.529 | 33847_s_at | cyclin-dependent kinase | CDKN1B (SEQ ID NO: 27) |
| | | | inhibitor 1B (p27, Kip1) | |
| 14 | 1.528 | 38663_at | barrier to autointegration | BANF1 (SEQ ID NO: 28) |
| | | | factor 1 | |
| 7 | 1.526 | 38568_at | topoisomerase I binding, | TOPORS (SEQ ID NO: 29) |
| | | | arginine/serine-rich | |
| 19 | 1.523 | 37162_at | coiled-coil domain | CCDC6 (SEQ ID NO: 30) |
| | | | containing 6 | |
| 76 | 1.503 | 39731_at | RNA binding motif protein, | RBMX (SEQ ID NO: 31) |
| | | | X-linked | |
| 89 | 1.493 | 38674_at | KIAA1354 protein | KIAA1354 (SEQ ID NO: 32) |
| 32 | 1.489 | 32219_at | tousled-like kinase 1 | TLK1 (SEQ ID NO: 33) |
| 46 | 1.479 | 36980_at | proline-rich nuclear | PNRC1 (SEQ ID NO: 34) |
| | | | receptor coactivator 1 | |
| 41 | 1.478 | 38581_at | Clone IMAGE: 5289004, | |
| | | | mRNA | |
| 92 | 1.453 | 1258_s_at | excision repair cross- | ERCC4 (SEQ ID NO: 35) |
| | | | complementing rodent | |
| | | | repair deficiency, | |
| | | | complementation group 4 | |
| 16 | 1.45 | 37316_r_at | chromosome 14 open | C14orf11 (SEQ ID NO: 36) |
| | | | reading frame 11 | |
| 3 | 1.443 | 37994_at | fragile X mental | FMR1 (SEQ ID NO: 37) |
| | | | retardation 1 | |
| 81 | 1.429 | 40457_at | splicing factor, | SFRS3 (SEQ ID NO: 38) |
| | | | arginine/serine-rich 3 | |
| 45 | 1.427 | 41152_f_at | ribosomal protein L36a- | RPL36AL (SEQ ID NO: 39) |
| | | | like | |
| 42 | 1.423 | 32752_at | NADH dehydrogenase | NDUFA7 (SEQ ID NO: 40) |
| | | | (ubiquinone) 1 alpha | |
| | | | subcomplex, 7, 14.5 kDa | |
| 35 | 1.416 | 41533_at | hypothetical protein | MGC39325 (SEQ ID |
| | | | MGC39325 | NO: 41) |
| 85 | 1.412 | 38659_at | soc-2 suppressor of clear | SHOC2 (SEQ ID NO: 42) |
| | | | homolog (C. elegans) | |
| 30 | 1.404 | 36571_at | topoisomerase (DNA) II | TOP2B (SEQ ID NO: 43) |
| | | | beta 180 kDa | |
| 17 | 1.401 | 38368_at | dUTP pyrophosphatase | DUT (SEQ ID NO: 44) |
| 94 | 1.396 | 35677_at | hypothetical protein | MGC9084 (SEQ ID NO: 45) |
| | | | MGC9084 | |
| 93 | 1.394 | 40086_at | KIAA0261 | KIAA0261 (SEQ ID NO: 46) |
| 67 | 1.377 | 39091_at | cytoskeleton related | JWA (SEQ ID NO: 47) |
| | | | vitamin A responsive | |
| | | | protein | |
| 75 | 1.373 | 40045_g_at | chromosome 18 open | C18orf1 (SEQ ID NO: 48) |
| | | | reading frame 1 | |
| 54 | 1.368 | 38892_at | KIAA0240 | KIAA0240 (SEQ ID NO: 49) |
| 47 | 1.358 | 34699_at | CD2-associated protein | CD2AP (SEQ ID NO: 50) |
| 64 | 1.337 | 32857_at | Ras association | RASSF3 (SEQ ID NO: 51) |
| | | | (RalGDS/AF-6) domain | |
| | | | family 3 | |
| 55 | 1.31 | 38119_at | glycophorin C (Gerbich | GYPC (SEQ ID NO: 52) |
| | | | blood group) | |
| 63 | 1.31 | 36033_at | splicing factor, | SFRS12 (SEQ ID NO: 53) |
| | | | arginine/serine-rich 12 | |
| 86 | 1.306 | 41343_at | CDP-diacylglycerol | CDS2 (SEQ ID NO: 54) |
| | | | synthase (phosphatidate | |
| | | | cytidylyltransferase) 2 | |
| 72 | 1.301 | 41573_at | Sp3 transcription factor | SP3 (SEQ ID NO: 55) |
| 70 | 1.296 | 35739_at | myotubularin related | MTMR3 (SEQ ID NO: 56) |
| | | | protein 3 | |
| 58 | 1.287 | 41457_at | KIAA0423 | KIAA0423 (SEQ ID NO: 57) |
| 82 | 1.283 | 32169_at | F-box only protein 21 | FBXO21 (SEQ ID NO: 58) |
| 50 | 1.276 | 41595_at | KIAA0947 protein | KIAA0947 (SEQ ID NO: 59) |
| 78 | 1.274 | 33348_at | transcription factor 12 | TCF12 (SEQ ID NO: 60) |
| | | | (HTF4, helix-loop-helix | |
| | | | transcription factors 4) | |
| 74 | 1.248 | 37361_at | fibroblast growth factor | FIBP (SEQ ID NO: 61) |
| | | | (acidic) intracellular | |
| | | | binding protein | |
| 62 | 0.767 | 882_at | colony stimulating factor 1 | CSF1 (SEQ ID NO: 62) |
| | | | (macrophage) | |
| 60 | 0.754 | 35309_at | suppression of | ST14 (SEQ ID NO: 63) |
| | | | tumorigenicity 14 (colon | |
| | | | carcinoma, matriptase, | |
| | | | epithin) | |
| 22 | 0.751 | 33863_at | hypoxia up-regulated 1 | HYOU1 (SEQ ID NO: 64) |
| 56 | 0.739 | 38281_at | caspase 7, apoptosis- | CASP7 (SEQ ID NO: 65) |
| | | | related cysteine protease | |
| 39 | 0.737 | 32566_at | chondroitin polymerizing | CHPF (SEQ ID NO: 66) |
| | | | factor | |
| 53 | 0.711 | 40237_at | pleckstrin homology-like | PHLDA2 (SEQ ID NO: 67) |
| | | | domain, family A, member 2 | |
| 98 | 0.704 | 1933_g_at | ATP-binding cassette, | ABCC5 (SEQ ID NO: 68) |
| | | | sub-family C | |
| | | | (CFTR/MRP), member 5 | |
| 37 | 0.699 | 33212_at | ribosome binding protein 1 | RRBP1 (SEQ ID NO: 69) |
| | | | homolog 180 kDa (dog) | |
| 77 | 0.698 | 1644_at | eukaryotic translation | EIF3S2 (SEQ ID NO: 70) |
| | | | initiation factor 3, subunit | |
| | | | 2 beta, 36 kDa | |
| 27 | 0.681 | 33667_at | peptidylprolyl isomerase A | PPIA (SEQ ID NO: 71) |
| | | | (cyclophilin A) | |
| 18 | 0.672 | 38760_f_at | butyrophilin, subfamily 3, | BTN3A2 (SEQ ID NO: 72) |
| | | | member A2 | |
| 43 | 0.671 | 38969_at | chromosome 19 open | C19orf10 (SEQ ID NO: 73) |
| | | | reading frame 10 | |
| 69 | 0.651 | 40116_at | phosphofructokinase, liver | PFKL (SEQ ID NO: 74) |
| 97 | 0.63 | 40164_at | Rho GDP dissociation | ARHGDIA (SEQ ID NO: 75) |
| | | | inhibitor (GDI) alpha | |
| 33 | 0.615 | 32116_at | epidermodysplasia | EVER1 (SEQ ID NO: 76) |
| | | | verruciformis 1 | |
| 49 | 0.614 | 32332_at | isocitrate dehydrogenase | IDH2 (SEQ ID NO: 77) |
| | | | 2 (NADP+), mitochondrial | |
| 9 | 0.589 | 41220_at | MLL septin-like fusion | MSF (SEQ ID NO: 78) |
| 8 | 0.57 | 40290_f_at | sialyltransferase 4A (beta- | SIAT4A (SEQ ID NO: 79) |
| | | | galactoside alpha-2,3- | |
| | | | sialyltransferase) | |
| 5 | 0.569 | 32378_at | pyruvate kinase, muscle | PKM2 (SEQ ID NO: 80) |
| 6 | 0.562 | 36614_at | heat shock 70 kDa protein | HSPA5 (SEQ ID NO: 81) |
| | | | 5 (glucose-regulated | |
| | | | protein, 78 kDa) | |
| 34 | 0.54 | 34885_at | synaptogyrin 2 | SYNGR2 (SEQ ID NO: 82) |
| 90 | 0.535 | 37920_at | paired-like homeodomain | PITX1 (SEQ ID NO: 83) |
| | | | transcription factor 1 | |
| 44 | 0.501 | 31960_f_at | G antigen 2 | GAGE2 (SEQ ID NO: 84) |
| 84 | 0.495 | 37383_f_at | major histocompatibility | HLA-C (SEQ ID NO: 85) |
| | | | complex, class I, C | |
| 95 | 0.49 | 37741_at | pyrroline-5-carboxylate | PYCR1 (SEQ ID NO: 86) |
| | | | reductase 1 | |
| 20 | 0.487 | 39708_at | signal transducer and | STAT3 (SEQ ID NO: 87) |
| | | | activator of transcription 3 | |
| | | | (acute-phase response | |
| | | | factor) | |
| 73 | 0.471 | 32174_at | solute carrier family 9 | SLC9A3R1 (also referred to |
| | | | (sodium/hydrogen | as EBP50; SEQ ID NO: 88) |
| | | | exchanger), isoform 3 | |
| | | | regulator 1 | |
| 1 | 0.45 | 41045_at | secreted and | SECTM1 (SEQ ID NO: 89) |
| | | | transmembrane 1 | |
| 80 | 0.395 | 36454_at | carbonic anhydrase XII | CA12 (SEQ ID NO: 90) |
| 11 | 0.294 | 35174_i_at | eukaryotic translation | EEF1A2 (SEQ ID NO: 91) |
| | | | elongation factor 1 alpha 2 | |
| 91 | 0.29 | 39781_at | insulin-like growth factor | IGFBP4 (SEQ ID NO: 92) |
| | | | binding protein 4 | |
| 38 | 0.281 | 1737_s_at | insulin-like growth factor | IGFBP4 (SEQ ID NO: 92) |
| | | | binding protein 4 | |
| 13 | 0.195 | 36681_at | apolipoprotein D | APOD (SEQ ID NO: 93) |

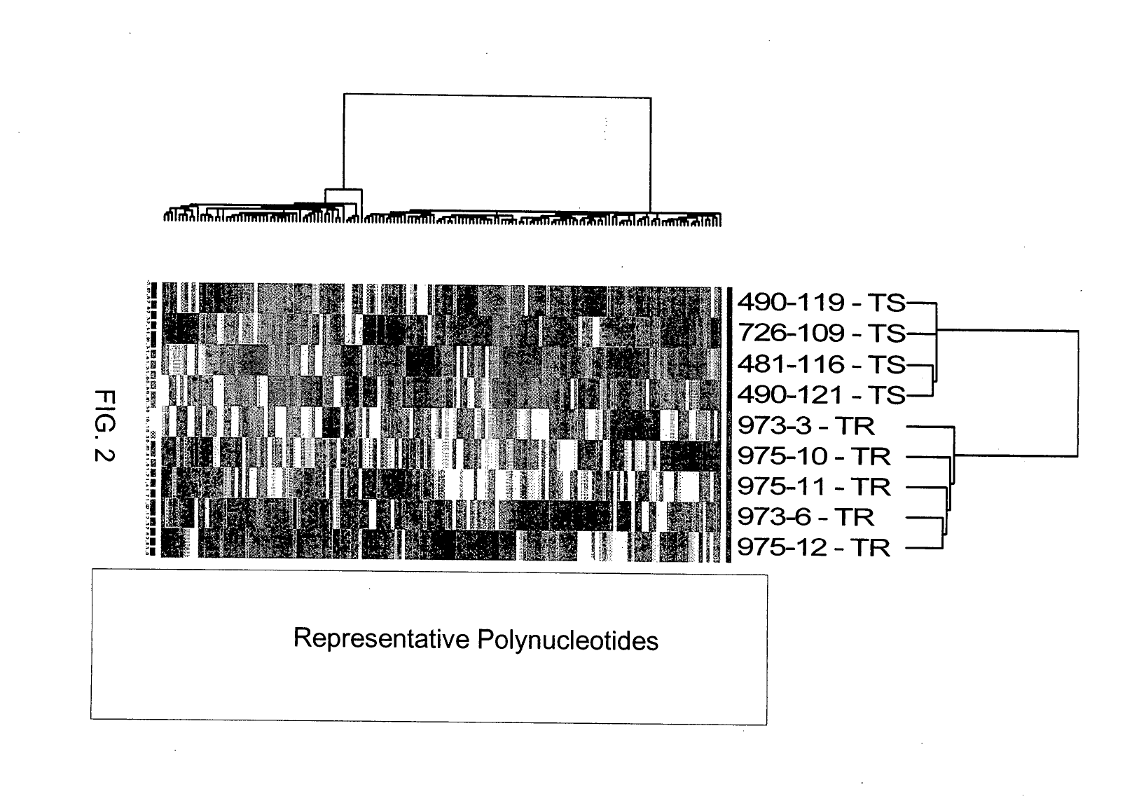

FIG. 2 shows an exemplary cluster at p=0.05 from the inventive microarray analysis, and Table 2 lists characteristic genes (n=155) identified at this level of significance. The genes are listed in no particular order, and exemplary sequences are provided in the accompanying sequence listing.

TABLE 2
Genes Identified by Tamoxifen Resistance Expression Analysis (p > 0.05)

| | Fold | | | |
|---|---|---|---|---|
| | difference | | | |
| | of geom | | | |
| | means | Probe set | Description | Gene symbol |
| 88 | 2.874 | 38317_at | transcription elongation | TCEAL1 (SEQ ID NO: 2) |
| | | | factor A (SII)-like 1 | |
| 320 | 2.762 | 36925_at | heat shock 70 kDa protein 2 | HSPA2 (SEQ ID NO: 94) |
| 10 | 2.345 | 37221_at | protein kinase, cAMP- | PRKAR2B (SEQ ID NO: 4) |
| | | | dependent, regulatory, | |
| | | | type II, beta | |
| 155 | 2.304 | 843_at | protein tyrosine | PTP4A1 (SEQ ID NO: 95) |
| | | | phosphatase type IVA, | |
| | | | member 1 | |
| 4 | 2.182 | 39072_at | MAX interactor 1 | MXI1 (SEQ ID NO: 6) |
| 12 | 2.106 | 31792_at | annexin A3 | ANXA3 (SEQ ID NO: 7) |
| 23 | 2.103 | 1577_at | androgen receptor | AR (SEQ ID NO: 8) |
| | | | (dihydrotestosterone | |
| | | | receptor; testicular | |
| | | | feminization; spinal and | |
| | | | bulbar muscular atrophy; | |
| | | | Kennedy disease) | |
| 24 | 2.057 | 39032_at | transforming growth factor | TGFB1I4 (SEQ ID NO: 9) |
| | | | beta 1 induced transcript 4 | |
| 96 | 2.04 | 654_at | MAX interactor 1 | MXI1 (SEQ ID NO: 6) |
| 135 | 1.942 | 1295_at | v-rel reticuloendotheliosis | RELA (SEQ ID NO: 96) |
| | | | viral oncogene homolog A, | |
| | | | nuclear factor of kappa | |
| | | | light polypeptide gene | |
| | | | enhancer in B-cells 3, p65 | |
| | | | (avian) | |
| 26 | 1.897 | 1237_at | immediate early response 3 | IER3 (SEQ ID NO: 10) |
| 127 | 1.881 | 36645_at | v-rel reticuloendotheliosis | RELA (SEQ ID NO: 96) |
| | | | viral oncogene homolog A, | |
| | | | nuclear factor of kappa | |
| | | | light polypeptide gene | |
| | | | enhancer in B-cells 3, p65 | |
| | | | (avian) | |
| 183 | 1.877 | 38985_at | leptin receptor overlapping | LEPROTL1 (SEQ ID NO: 97) |
| | | | transcript-like 1 | |
| 87 | 1.836 | 38375_at | esterase | ESD (SEQ ID NO: 11) |
| | | | D/formylglutathione | |
| | | | hydrolase | |
| 119 | 1.763 | 37908_at | guanine nucleotide | GNG11 (SEQ ID NO: 98) |
| | | | binding protein (G | |
| | | | protein), gamma 11 | |
| 52 | 1.688 | 1909_at | B-cell CLL/lymphoma 2 | BCL2 (SEQ ID NO: 14) |
| 122 | 1.688 | 40868_at | hypothetical protein | FLJ20274 (SEQ ID NO: 99) |
| | | | FLJ20274 | |
| 21 | 1.687 | 41690_at | AT rich interactive domain | ARID5B (SEQ ID NO: 15) |
| | | | 5B (MRF1-like) | |
| 57 | 1.684 | 41141_at | Protein-kinase, interferon- | PRKRIR (SEQ ID NO: 16) |
| | | | inducible double stranded | |
| | | | RNA dependent inhibitor, | |
| | | | repressor of (P58 | |
| | | | repressor) | |
| 68 | 1.672 | 31800_at | MRNA; cDNA | |
| | | | DKFZp586L141 (from | |
| | | | clone DKFZp586L141) | |
| 25 | 1.666 | 40838_at | zinc finger protein 292 | ZNF292 (SEQ ID NO: 17) |
| 79 | 1.655 | 36097_at | immediate early response 2 | IER2 (SEQ ID NO: 18) |
| 111 | 1.624 | 36514_at | cell growth regulator with | CGRRF1 (SEQ ID NO: 100) |
| | | | ring finger domain 1 | |
| 151 | 1.62 | 2036_s_at | CD44 antigen (homing | CD44 (SEQ ID NO: 101) |
| | | | function and Indian blood | |
| | | | group system) | |
| 51 | 1.587 | 34819_at | CD164 antigen, | CD164 (SEQ ID NO: 19) |
| | | | sialomucin | |
| 66 | 1.579 | 38764_at | Dicer1, Dcr-1 homolog | DICER1 (SEQ ID NO: 20) |
| | | | (Drosophila) | |
| 29 | 1.559 | 37661_at | ATPase, Ca++ | ATP2B1 (SEQ ID NO: 23) |
| | | | transporting, plasma | |
| | | | membrane 1 | |
| 31 | 1.542 | 39028_at | karyopherin (importin) | KPNB3 (SEQ ID NO: 24) |
| | | | beta 3 | |
| 188 | 1.542 | 41542_at | zinc finger protein 216 | ZNF216 (SEQ ID NO: 102) |
| 303 | 1.542 | 40859_at | nuclear protein UKp68 | FLJ11806 (SEQ ID NO: 103) |
| 2 | 1.541 | 38049_g_at | RNA binding protein with | RBPMS (SEQ ID NO: 25) |
| | | | multiple splicing | |
| 28 | 1.54 | 38765_at | Dicer1, Dcr-1 homolog | DICER1 (SEQ ID NO: 20) |
| | | | (Drosophila) | |
| 15 | 1.531 | 38242_at | B-cell linker | BLNK (SEQ ID NO: 26) |
| 14 | 1.528 | 38663_at | barrier to autointegration | BANF1 (SEQ ID NO: 28) |
| | | | factor 1 | |
| 7 | 1.526 | 38568_at | topoisomerase I binding, | TOPORS (SEQ ID NO: 29) |
| | | | arginine/serine-rich | |
| 19 | 1.523 | 37162_at | coiled-coil domain | CCDC6 (SEQ ID NO: 30) |
| | | | containing 6 | |
| 113 | 1.521 | 40066_at | ubiquitin-activating | UBE1C (SEQ ID NO: 104) |
| | | | enzyme E1C (UBA3 | |
| | | | homolog, yeast) | |
| 179 | 1.518 | 39806_at | ASF1 anti-silencing | ASF1A (SEQ ID NO: 105) |
| | | | function 1 homolog A (S. cerevisiae) | |
| 125 | 1.509 | 37723_at | cyclin G2 | CCNG2 (SEQ ID NO: 106) |
| 32 | 1.489 | 32219_at | tousled-like kinase 1 | TLK1 (SEQ ID NO: 33) |
| 120 | 1.482 | 34445_at | expressed in | HHL (SEQ ID NO: 107) |
| | | | hematopoietic cells, heart, | |
| | | | liver | |
| 41 | 1.478 | 38581_at | Clone IMAGE: 5289004, | |
| | | | mRNA | |
| 176 | 1.476 | 39132_at | SWI/SNF related, matrix | SMARCA5 (SEQ ID |
| | | | associated, actin | NO: 108) |
| | | | dependent regulator of | |
| | | | chromatin, subfamily a, | |
| | | | member 5 | |
| 92 | 1.453 | 1258_s_at | excision repair cross- | ERCC4 (SEQ ID NO: 35) |
| | | | complementing rodent | |
| | | | repair deficiency, | |
| | | | complementation group 4 | |
| 336 | 1.452 | 1817_at | prefoldin 5 | PFDN5 (SEQ ID NO: 109) |
| 100 | 1.451 | 32165_at | splicing factor, | SFRS7 (SEQ ID NO: 110) |
| | | | arginine/serine-rich 7, | |
| | | | 35 kDa | |
| 16 | 1.45 | 37316_r_at | chromosome 14 open | C14orf11 (SEQ ID NO: 36) |
| | | | reading frame 11 | |
| 3 | 1.443 | 37994_at | fragile × mental | FMR1 (SEQ ID NO: 37) |
| | | | retardation 1 | |
| 45 | 1.427 | 41152_f_at | ribosomal protein L36a- | RPL36AL (SEQ ID NO: 39) |
| | | | like | |
| 42 | 1.423 | 32752_at | NADH dehydrogenase | NDUFA7 (SEQ ID NO: 40) |
| | | | (ubiquinone) 1 alpha | |
| | | | subcomplex, 7, 14.5 kDa | |
| 35 | 1.416 | 41533_at | hypothetical protein | MGC39325 (SEQ ID NO: 41) |
| | | | MGC39325 | |
| 85 | 1.412 | 38659_at | soc-2 suppressor of clear | SHOC2 (SEQ ID NO: 42) |
| | | | homolog (C. elegans) | |
| 123 | 1.41 | 36857_at | RAD1 homolog (S. pombe) | RAD1 (SEQ ID NO: 111) |
| 30 | 1.404 | 36571_at | topoisomerase (DNA) II | TOP2B (SEQ ID NO: 43) |
| | | | beta 180 kDa | |
| 17 | 1.401 | 38368_at | dUTP pyrophosphatase | DUT (SEQ ID NO: 44) |
| 94 | 1.396 | 35677_at | hypothetical protein | MGC9084 (SEQ ID NO: 112) |
| | | | MGC9084 | |
| 124 | 1.395 | 33378_at | IDN3 protein | IDN3 (SEQ ID NO: 113) |
| 93 | 1.394 | 40086_at | KIAA0261 | KIAA0261 (SEQ ID NO: 114) |
| 350 | 1.392 | 1882_g_at | | |
| 146 | 1.384 | 40610_at | zinc finger RNA binding | ZFR (SEQ ID NO: 115) |
| | | | protein | |
| 67 | 1.377 | 39091_at | cytoskeleton related | JWA (SEQ ID NO: 47) |
| | | | vitamin A responsive | |
| | | | protein | |
| 75 | 1.373 | 40045_g_at | chromosome 18 open | C18orf1 (SEQ ID NO: 48) |
| | | | reading frame 1 | |
| 54 | 1.368 | 38892_at | KIAA0240 | KIAA0240 (SEQ ID NO: 49) |
| 258 | 1.365 | 41131_f_at | heterogeneous nuclear | HNRPH2 (SEQ ID NO: 116) |
| | | | ribonucleoprotein H2 (H′) | |
| 236 | 1.364 | 41503_at | zinc fingers and | ZHX2 (SEQ ID NO: 117) |
| | | | homeoboxes 2 | |
| 47 | 1.358 | 34699_at | CD2-associated protein | CD2AP (SEQ ID NO: 50) |
| 64 | 1.337 | 32857_at | Ras association | RASSF3 (SEQ ID NO: 51) |
| | | | (RalGDS/AF-6) domain | |
| | | | family 3 | |
| 300 | 1.335 | 35991_at | LSM6 homolog, U6 small | LSM6 (SEQ ID NO: 118) |
| | | | nuclear RNA associated | |
| | | | (S. cerevisiae) | |
| 197 | 1.319 | 763_at | glia maturation factor, beta | GMFB (SEQ ID NO: 119) |
| 55 | 1.31 | 38119_at | glycophorin C (Gerbich | GYPC (SEQ ID NO: 52) |
| | | | blood group) | |
| 63 | 1.31 | 36033_at | splicing factor, | SFRS12 (SEQ ID NO: 120) |
| | | | arginine/serine-rich 12 | |
| 86 | 1.306 | 41343_at | CDP-diacylglycerol | CDS2 (SEQ ID NO: 54) |
| | | | synthase (phosphatidate | |
| | | | cytidylyltransferase) 2 | |
| 72 | 1.301 | 41573_at | Sp3 transcription factor | SP3 (SEQ ID NO: 55) |
| 102 | 1.3 | 35838_at | zinc finger protein 410 | ZNF410 (SEQ ID NO: 121) |
| 112 | 1.297 | 32539_at | COP9 constitutive | COPS8 (SEQ ID NO: 122) |
| | | | photomorphogenic | |
| | | | homolog subunit 8 | |
| | | | (Arabidopsis) | |
| 70 | 1.296 | 35739_at | myotubularin related | MTMR3 (SEQ ID NO: 56) |
| | | | protein 3 | |
| 115 | 1.292 | 39774_at | oxidase (cytochrome c) | OXA1L (SEQ ID NO: 123) |
| | | | assembly 1-like | |
| 58 | 1.287 | 41457_at | KIAA0423 | KIAA0423 (SEQ ID NO: 57) |
| 198 | 1.284 | 35019_at | zinc finger protein 539 | ZNF539 (SEQ ID NO: 124) |
| 82 | 1.283 | 32169_at | F-box only protein 21 | FBXO21 (SEQ ID NO: 58) |
| 187 | 1.282 | 36164_at | pyruvate dehydrogenase | PDHX (SEQ ID NO: 125) |
| | | | complex, component X | |
| 50 | 1.276 | 41595_at | KIAA0947 protein | KIAA0947 (SEQ ID NO: 59) |
| 78 | 1.274 | 33348_at | transcription factor 12 | TCF12 (SEQ ID NO: 60) |
| | | | (HTF4, helix-loop-helix | |
| | | | transcription factors 4) | |
| 181 | 1.274 | 37826_at | ubiquitin-conjugating | UBE2D1 (SEQ ID NO: 126) |
| | | | enzyme E2D 1 (UBC4/5 | |
| | | | homolog, yeast) | |
| 134 | 1.263 | 35255_at | importin 7 | IPO7 (SEQ ID NO: 127) |
| 363 | 1.259 | 37690_at | ilvB (bacterial acetolactate | ILVBL (SEQ ID NO: 128) |
| | | | synthase)-like | |
| 126 | 1.251 | 38654_at | heterogeneous nuclear | HNRPU (SEQ ID NO: 129) |
| | | | ribonucleoprotein U | |
| | | | (scaffold attachment factor | |
| | | | A) | |
| 74 | 1.248 | 37361_at | fibroblast growth factor | FIBP (SEQ ID NO: 61) |
| | | | (acidic) intracellular | |
| | | | binding protein | |
| 171 | 1.246 | 40515_at | eukaryotic translation | EIF2B2 (SEQ ID NO: 130) |
| | | | initiation factor 2B, subunit | |
| | | | 2 beta, 39 kDa | |
| 165 | 1.231 | 36463_at | BCL2-associated | BAG5 (SEQ ID NO: 131) |
| | | | athanogene 5 | |
| 148 | 1.229 | 31879_at | far upstream element | FUBP3 (SEQ ID NO: 132) |
| | | | (FUSE) binding protein 3 | |
| 170 | 1.221 | 41628_at | fucosyltransferase 8 | FUT8 (SEQ ID NO: 133) |
| | | | (alpha (1,6) | |
| | | | fucosyltransferase) | |
| 325 | 1.215 | 842_at | protein kinase C binding | PRKCBP1 (SEQ ID |
| | | | protein 1 | NO: 134) |
| 385 | 1.202 | 1695_at | neural precursor cell | NEDD8 (SEQ ID NO: 135) |
| | | | expressed, | |
| | | | developmentally down- | |
| | | | regulated 8 | |
| 218 | 1.2 | 33472_at | flavin containing | FMO4 (SEQ ID NO: 136) |
| | | | monooxygenase 4 | |
| 335 | 1.193 | 37725_at | protein phosphatase 1, | PPP1CC (SEQ ID NO: 137) |
| | | | catalytic subunit, gamma | |
| | | | isoform | |
| 212 | 1.186 | 37077_at | pyruvate kinase, liver and | PKLR (SEQ ID NO: 138) |
| | | | RBC | |
| 238 | 1.186 | 751_at | phosphatidylinositol | PIGC (SEQ ID NO: 139) |
| | | | glycan, class C | |
| 327 | 1.171 | 38390_at | component of oligomeric | COG2 (SEQ ID NO: 140) |
| | | | golgi complex 2 | |
| 393 | 1.169 | 33883_at | embryonal Fyn-associated | EFS (SEQ ID NO: 141) |
| | | | substrate | |
| 433 | 0.864 | 39157_at | immunoglobulin lambda | IGLVIVOR22-2 |
| | | | variable (IV)/OR22-2 | |
| 331 | 0.851 | 35850_at | phosphatidylserine | PTDSR (SEQ ID NO: 142) |
| | | | receptor | |
| 333 | 0.848 | 33169_at | neogenin homolog 1 | NEO1 (SEQ ID NO: 143) |
| | | | (chicken) | |
| 318 | 0.847 | 32660_at | KIAA0342 gene product | KIAA0342 (SEQ ID NO: 144) |
| 250 | 0.845 | 189_s_at | plasminogen activator, | PLAUR (SEQ ID NO: 145) |
| | | | urokinase receptor | |
| 312 | 0.845 | 33441_at | T-cell leukemia | TCTA (SEQ ID NO: 146) |
| | | | translocation altered gene | |
| 347 | 0.844 | 1277_at | Rho guanine exchange | ARHGEF16 (SEQ ID |
| | | | factor (GEF) 16 | NO: 147) |
| 223 | 0.84 | 39432_at | UDP-Gal: betaGlcNAc beta | B4GALT4 (SEQ ID NO: 148) |
| | | | 1,4-galactosyltransferase, | |
| | | | polypeptide 4 | |
| 421 | 0.837 | 36218_g_at | serine/threonine kinase 38 | STK38 (SEQ ID NO: 149) |
| 286 | 0.831 | 1014_at | polymerase (DNA | POLG (SEQ ID NO: 150) |
| | | | directed), gamma | |
| 285 | 0.812 | 38832_r_at | guanine nucleotide | GNB2 (SEQ ID NO: 151) |
| | | | binding protein (G | |
| | | | protein), beta polypeptide 2 | |
| 329 | 0.809 | 41162_at | protein phosphatase 1G | PPM1G (SEQ ID NO: 152) |
| | | | (formerly 2C), | |
| | | | magnesium-dependent, | |
| | | | gamma isoform | |
| 334 | 0.808 | 40329_at | solute carrier family 39 | SLC39A7 (SEQ ID NO: 153) |
| | | | (zinc transporter), member 7 | |
| 169 | 0.805 | 40083_at | senataxin | KIAA0625 (SEQ ID NO: 154) |
| 175 | 0.795 | 40127_at | paired related homeobox 1 | PRRX1 (SEQ ID NO: 155) |
| 114 | 0.781 | 35630_at | lethal giant larvae | LLGL2 (SEQ ID NO: 156) |
| | | | homolog 2 (Drosophila) | |
| 62 | 0.767 | 882_at | colony stimulating factor 1 | CSF1 (SEQ ID NO: 62) |
| | | | (macrophage) | |
| 130 | 0.766 | 31953_f_at | G antigen 3 | GAGE3 (SEQ ID NO: 157) |
| 60 | 0.754 | 35309_at | suppression of | ST14 (SEQ ID NO: 63) |
| | | | tumorigenicity 14 (colon | |
| | | | carcinoma, matriptase, | |
| | | | epithin) | |
| 22 | 0.751 | 33863_at | hypoxia up-regulated 1 | HYOU1 (SEQ ID NO: 64) |
| 39 | 0.737 | 32566_at | chondroitin polymerizing | CHPF (SEQ ID NO: 66) |
| | | | factor | |
| 219 | 0.733 | 41742_s_at | optineurin | OPTN (SEQ ID NO: 158) |
| 192 | 0.73 | 36963_at | phosphogluconate | PGD (SEQ ID NO: 159) |
| | | | dehydrogenase | |
| 53 | 0.711 | 40237_at | pleckstrin homology-like | PHLDA2 (SEQ ID NO: 67) |
| | | | domain, family A, member 2 | |
| 98 | 0.704 | 1933_g_at | ATP-binding cassette, | ABCC5 (SEQ ID NO: 68) |
| | | | sub-family C | |
| | | | (CFTR/MRP), member 5 | |
| 104 | 0.701 | 41231_f_at | high-mobility group | HMGN2 (SEQ ID NO: 160 |
| | | | nucleosomal binding | |
| | | | domain 2 | |
| 37 | 0.699 | 33212_at | ribosome binding protein 1 | RRBP1 (SEQ ID NO: 69) |
| | | | homolog 180 kDa (dog) | |
| 77 | 0.698 | 1644_at | eukaryotic translation | EIF3S2 (SEQ ID NO: 70) |
| | | | initiation factor 3, subunit | |
| | | | 2 beta, 36 kDa | |
| 27 | 0.681 | 33667_at | peptidylprolyl isomerase A | PPIA (SEQ ID NO: 71) |
| | | | (cyclophilin A) | |
| 251 | 0.678 | 37585_at | small nuclear | SNRPA1 (SEQ ID NO: 161 |
| | | | ribonucleoprotein | |
| | | | polypeptide A′ | |
| 18 | 0.672 | 38760_f_at | butyrophilin, subfamily 3, | BTN3A2 (SEQ ID NO: 72) |
| | | | member A2 | |
| 178 | 0.652 | 34264_at | RUN and SH3 domain | RUSC1 (SEQ ID NO: 162 |
| | | | containing 1 | |
| 150 | 0.652 | 32275_at | secretory leukocyte | SLPI (SEQ ID NO: 163 |
| | | | protease inhibitor | |
| | | | (antileukoproteinase) | |
| 69 | 0.651 | 40116_at | phosphofructokinase, liver | PFKL (SEQ ID NO: 74) |
| 33 | 0.615 | 32116_at | epidermodysplasia | EVER1 (SEQ ID NO: 76) |
| | | | verruciformis 1 | |
| 49 | 0.614 | 32332_at | isocitrate dehydrogenase | IDH2 (SEQ ID NO: 77) |
| | | | 2 (NADP+), mitochondrial | |
| 180 | 0.611 | 38790_at | epoxide hydrolase 1, | EPHX1 (SEQ ID NO: 164 |
| | | | microsomal (xenobiotic) | |
| 301 | 0.596 | 34703_f_at | | |
| 9 | 0.589 | 41220_at | MLL septin-like fusion | MSF (SEQ ID NO: 78) |
| 434 | 0.589 | 1180_g_at | | |
| 8 | 0.57 | 40290_f_at | sialyltransferase 4A (beta- | SIAT4A (SEQ ID NO: 79) |
| | | | galactoside alpha-2,3- | |
| | | | sialyltransferase) | |
| 5 | 0.569 | 32378_at | pyruvate kinase, muscle | PKM2 (SEQ ID NO: 80) |
| 6 | 0.562 | 36614_at | heat shock 70 kDa protein | HSPA5 (SEQ ID NO: 81) |
| | | | 5 (glucose-regulated | |
| | | | protein, 78 kDa) | |
| 34 | 0.54 | 34885_at | synaptogyrin 2 | SYNGR2 (SEQ ID NO: 82) |
| 90 | 0.535 | 37920_at | paired-like homeodomain | PITX1 (SEQ ID NO: 83) |
| | | | transcription factor 1 | |
| 44 | 0.501 | 31960_f_at | G antigen 2 | GAGE2 (SEQ ID NO: 84) |
| 168 | 0.491 | 691_g_at | procollagen-proline, 2- | P4HB (SEQ ID NO: 165 |
| | | | oxoglutarate 4- | |
| | | | dioxygenase (proline 4- | |
| | | | hydroxylase), beta | |
| | | | polypeptide (protein | |
| | | | disulfide isomerase; | |
| | | | thyroid hormone binding | |
| | | | protein p55) | |
| 20 | 0.487 | 39708_at | signal transducer and | STAT3 (SEQ ID NO: 87) |
| | | | activator of transcription 3 | |
| | | | (acute-phase response | |
| | | | factor) | |
| 73 | 0.471 | 32174_at | solute carrier family 9 | SLC9A3R1 (also referred to |
| | | | (sodium/hydrogen | as EBP50; SEQ ID NO: 88) |
| | | | exchanger), isoform 3 | |
| | | | regulator 1 | |
| 1 | 0.45 | 41045_at | secreted and | SECTM1 (SEQ ID NO: 89) |
| | | | transmembrane 1 | |
| 80 | 0.395 | 36454_at | carbonic anhydrase XII | CA12 (SEQ ID NO: 90) |
| 153 | 0.337 | 608_at | apolipoprotein E | APOE (SEQ ID NO: 166 |
| 11 | 0.294 | 35174_i_at | eukaryotic translation | EEF1A2 (SEQ ID NO: 91) |
| | | | elongation factor 1 alpha 2 | |
| 38 | 0.281 | 1737_s_at | insulin-like growth factor | IGFBP4 (SEQ ID NO: 92) |
| | | | binding protein 4 | |
| 13 | 0.195 | 36681_at | apolipoprotein D | APOD (SEQ ID NO: 93) |

In specific embodiments, there may be one or more expressed genes identified in Table 1 and/or Table 2 as associated with tamoxifen resistance and therefore is useful for predicting therapy for an individual. In additional embodiments, there may be combinations of expressed genes identified in Table 1 and/or Table 2 as being indicative of tamoxifen resistance and therefore predictive for therapy for an individual. There may be combinations of two expressed genes, three expressed genes, four expressed genes, or five or more expressed genes, for example. In specific embodiments, the profile comprises at least a phosphatase, such as, for example, DUSP6. In other embodiments, the profile additionally or alternative comprises EBP50 and/or RhoGDIa.

A skilled artisan recognizes that the relevance of the expressed genes indicative of tamoxifen resistance may be confirmed by routine methods in the art. For example, demonstration that overexpression of a particular gene confers resistance to tamoxifen in ER-positive breast cancer cells may employ genetic engineering, and then investigation of their growth in xenograft models of human breast cancer. Furthermore, inhibitors of the appropriate signaling pathways and/or siRNA knock-down studies to reduce the levels of potential ER accessory proteins or signaling molecules may be utilized. The development of small molecule inhibitors to resistance genes are ideal targets for drug development using rational drug design, in vitro chemical library screening on either a small or large scale, and high-content target-based cellular assays.

In specific embodiments, an expressed tamoxifen-resistant and/or tamoxifen-sensitive gene is assessed in an in vivo model system. For example, vectors comprising the expressed gene in question may be delivered to a xenograft breast cancer mouse model. Following this, the mouse are administered estrogen for a period of time, after which either tamoxifin or a control estrogen is delivered. If the gene is related to tamoxifen resistance, then the tumor size of transformed mice should increase. In vitro methods may also be employed to confirm association of a particular gene with tamoxifen resistance. For example, soft agar experiments well known in the art are useful for assessing colony number in the presence of the gene in question as a function of resistance to tamoxifen.

IV. Collection of Samples

In aspects of the invention, samples are obtained from an individual for subjecting to the methods, such as from an individual suspected of developing resistance to a chemotherapy. Any suitable methods for obtaining the samples are within the scope of the invention, and exemplary methods include by fine needle aspirates obtained via a biopsy procedure. Samples may be collected commensurate with the cancer for which the chemotherapy is directed, such as via a PAP smear, ductal lavage, fine needle aspiration, prostate massage, sputum (including saliva, bronchial brush or bronchial wash), stool, semen, urine, or other bodily fluid (including ascitic fluid, cerebral spinal fluid (CSF), bladder wash, and pleural fluid). Non-limiting examples of tissues susceptible to fine needle aspiration include lymph node, lung, thyroid, breast, and liver.

One or more cells of the samples may be isolated and used to prepare the RNA from said cell(s). In specific embodiments of the invention, the isolation of one or more cells may be performed by microdissection, such as, but not limited to, laser capture microdissection (LCM) or laser microdissection (LMD). The levels and/or activities of the RNA(s) may be assayed directly or indirectly, or may be amplified in whole or in part prior to detection.

V. Identification of Tumors

In specific embodiments of the invention, resistance to chemotherapy is determined in tumors that arise during the chemotherapy treatment by evaluating an RNA expression profile of expressed RNAs in the tumor(s). One aspect of this embodiment encompasses identifying the presence of tumors in the individual. This may be done by any suitable means in the art, including palpitation, sonogram, X-ray, biopsy, and so forth. In specific embodiments, the tumors are metastatic tumors. Following identification of the presence of one or more of the tumors, expressed RNA is isolated from one or more cells of the one or more tumors, and levels are determined, such as the levels of polynucleotides listed in Table 1, Table 2, or both.

VI. Estrogen Receptors and Prediction of Response to Therapy

It has been estimated that between 60-75% of women with invasive breast cancer express ER α and β forms (Harvey 1999; Fuqua, Schiff et al. 2003). ER α expression is an important prognostic factor predicting the natural history of breast cancer after surgery in the absence of treatment (Knight, Livingston et al. 1977; Clark and McGuire 1988). The prognostic value of ER α in breast cancer has not yet been firmly established, although the majority of studies suggest that like ER α, ERβ protein expression is also associated with a better outcome in untreated patients (Omoto, Kobayashi et al. 2002). It is well-established though that ER α levels predict a better response to Tam, and that response is directly related to the amount of receptor present in a tumor (Early_Breast_Cancer_Trialists'_Collaborative_Group 1998). Although it was initially hypothesized that ERβ expression might predict HR (Paech, Webb et al. 1997), it appears that high ERβ protein expression is actually associated with a better response to Tam in the majority of studies published to date (Mann, Laucirica et al. 2001; Iwase, Zhang et al. 2003)(Hopp, in press).

An important question is whether ER loss is a significant mechanism of acquired HR? Although ER α is reduced in Tam-resistant tumors overall, the development of HR is more frequently associated with the maintenance of ER α at the time of progression (Encamacion, Ciocca et al. 1993; Nedergaard, Haerslev et al. 1995; Kuukasjarvi, Kononen et al. 1996). Further support for the continued role of ER in HR comes from the use of other endocrine therapies with distinct mechanisms of action, such as the steroidal antagonist faslodex that exhibits no ER α agonist activity, in Tam-resistant (TR) patients. About two-thirds of TR breast cancer patients respond to second-line therapy with faslodex (Howell, DeFriend et al. 1996). Similarly, the third-generation aromatase inhibitors anastrazole and letrozole are most effective in post-menopausal TR patients (Buzdar, Douma et al. 2001). Thus, resistance to Tam does not result in global HR.

VII. Mechanisms of resistance to hormonal therapies

A. Proliferation

Undoubtedly, estrogen is important for the growth of many breast cancers. One explanation for the acquisition of HR could be the deregulated expression of cell cycle components which release the cell cycle and thus tumor proliferation from normal estrogen control. In fact, many of the estrogen-regulated genes identified in estrogen-responsive breast cancer cells are genes related to cell cycle regulation, such as cyclin A1, cyclin D1, a key regulator of the G1/S phase transition of the cell cycle, and the E2F1 transcription factor (Soulez and Parker 2001; Coser, Chesnes et al 2003; Hayashi, Eguchi et al. 2003). Tam also functions as an agonist to induce the expression of genes involved in promoting cell cycle progression, including fos, myc, cyclin A2, and E2F1 (Hodges, Cook et al. 2003). Interestingly, cyclin D1 is not directly induced by Tam treatment.

Cyclin D1 is a regulatory subunit for two cyclin-dependent kinases, cdk4 and cdk6. High levels of cyclin D1 in tumors may produce sufficient cdk4 activity that G1 progression occurs independently of normal controls, and it may titrate out Cip/kip inhibitors, making a cell insensitive to their negative regulation (Zhou, Hopp et al. 2001). Cyclin D1 can also stimulate ERα activity in the absence of estrogen (Neuman, Ladha et al. 1997; Zwijsen, Wientjens et al. 1997). Furthermore, cyclin D1 can form a complex with ERα and receptor coactivators (McMahon, Suthiphongchai et al. 1999; Lamb, Ladha et al. 2000), but there are conflicting results whether cyclin D1 overexpression affects Tam response in vitro. However, a recent retrospective study in patients with long-term clinical followup demonstrated that high cyclin D1 levels were associated with a worse overall survival in patients treated with Tam (Stendahl, Kronblad et al. 2004). Thus, although it remains to be validated, high cyclin D1 might be an independent predictor of HR.

B. Apoptosis

Tissue homeostasis is a fine balance between proliferation, apoptosis, and cellular differentiation. Apoptosis plays a key role in the growth regulation of normal and cancerous tissues, and its dysregulation can lead to cancer. We know that the withdrawal of hormones and/or growth factors can induce apoptosis in breast tissues, but there are relatively few studies that have examined the role of apoptosis following hormonal treatment of breast cancer. Tam induces apoptotic death in ERα-positive breast cancer cells, but there are number of apoptotic mechanisms which are believed to be non-ERα mediated (Mandlekar and Kong 2001). Estrogen treatment does increase the levels of the antiapoptotic proteins, bcl-2 and bclxL (Gompel, Somai et al. 2000), and ERα expression in breast tumors is strongly associated with bcl-2 levels (Simon 1993). The effects of Tam on apoptosis can also be reversed by the addition of estrogen (Gompel, Somai et al. 2000), suggesting that its effects could be mediated, in part, through apoptotic mechanisms. It has also been reported that overexpression of the HER2 growth factor receptor up-regulates the expression of bcl-2 and bcl-x1 in ERα-positive breast cancer cells, and suggest that bcl-2 may be associated with the relative TR of these engineered cell lines (Kumar, Mandal et al. 1996). However retrospective studies correlating increased bcl-2 levels with TR in clinical breast cancers are equivocal (Daidone, Luisi et al. 1999), demonstrating the need for more studies powered to address the question whether enhanced bcl-2 expression is present in HR tumors.

C. ER Mutations

A number of years ago, presented an attractive hypothesis that mutations in ERα itself might be found if ERα was acting as an “oncogene” during breast tumorigenesis (Fuqua 1994). However, unlike most oncogenes, it has been estimated that only 1% of primary tumors (Roodi, Bailey et al. 1995) exhibit missense mutations in the receptor. Karnik et al. (Karnik, Kulkami et al. 1994) reported that 1 of 5 metastatic breast tumors contained an ERα mutation, and found mutations in 3 of 30 metastatic lesions (Zhang, Borg et al. 1997), however the clinical evidence of these ERα mutations in metastatic tumors playing a role in HR is lacking, but it probably warrants further study given the small number of metastatic lesions that have been examined. In addition, these earlier studies were all performed on unselected, heterogenous tumor material before the introduction of laser-capture microdissection techniques.

The present inventors have utilized microdissection and manual genomic sequence analysis of DNA from premalignant breast lesions, and discovered an A to G transition at nucleotide 908 of ERα in approximately 30% of the lesions (Fuqua, Wiltschke et al. 2000 and U.S. Pat. No. 6,821,732). The mutation results in a substitution of lysine 303 for arginine (K303R ERα, and it increases the estrogen sensitivity and transcriptional activity of the receptor in breast cancer cells. The mutation also alters ERα binding to coactivators. The results indicate that the mutation is present in invasive breast tumors in US women (Fuqua 2002), but the mutation has not been detected in breast tumors from Japanese women (Zhang, Yamashita et al. 2003). It is tempting to speculate that this disparity could be related to the recognized lower incidence of ductal hyperplasia and tumor ERα-positivity in Japanese women (Stemmermann 1991), or other ethnic differences in etiology and incidence between the two countries (Maskarinec 2000; Deapen, Liu et al. 2002). However, the clinical significance, and any potential role that the K303R ERα mutation may have in HR is currently unknown.

D. Growth Factor Crosstalk

The role of growth factor crosstalk in HR is highlighted in data showing the impact of overexpression of components of the peptide growth factor signaling network (epidermal growth factor receptor (EGFR) and HER2) on the development of TR. It was first demonstrated that HER2 overexpression in ER+ MCF-7 human breast cancer xenografts renders them resistant to Tam (Benz, Scott et al. 1993), a finding substantiated by other groups who find markedly increased levels of EGFR and HER2 in Tam-resistant MCF-7 cells (Hutcheson, Knowlden et al. 2003; Nicholson, Gee et al. 2003). TR in breast cancer cells can also be reversed with EGFR/HER2 tyrosine kinase inhibitors, and combined treatment with Tam is even more effective (Kurokawa, Lenferink et al. 2000; Moulders, Yakes et al. 2001; Massarweh, Shou et al. 2002). The inventors have recently provided clinical evidence for the significance of this crosstalk in a prospective study showing a poorer disease-free survival for those patients receiving adjuvant tamoxifen whose tumors expressed high levels of both HER2 and the ER coregulatory protein AIB 1 (Osborne, Bardou et al. 2003). Thus, it can be concluded that growth factor receptor overexpression and continued ER expression are important for the development of TR, at least in the small portion of ER+ patients who co-express HER2 or EGFR.

The molecular basis for growth factor receptor-associated TR could involve enhanced downstream signal transduction, and current models advocate that growth factor stimulation of ER activity is likely to be mediated through phosphorylation of the ER. For instance, Mitogen Activated Protein Kinases (MAPK) ERK-1 and 2, which are downstream of growth factor signaling, have been shown to be biomarkers for a shorter duration of response to Tam in one small clinical study (Gee, Robertson et al. 2001). However; when MEK1, an upstream activator of MAPK, is overexpressed in MCF-7 cells, these cells remain sensitive to Tam (Atanaskova, Keshamouni et al. 2002). On the other hand, inhibition of MAPK activity can reverse TR in a HER2-overexpressing MCF-7 model system (Kurokawa, Lenferink et al. 2000). Thus, although it is apparent that MAPK activation can contribute to estrogen-induced proliferation (Castoria, Barone et al. 1999), estrogen sensitivity (Santen, Song et al. 2002), and cell survival (Razandi, Pedram et al. 2000), do not yet have a consensus on the molecular mechanisms which coordinate with MAPK activation and are required or involved in TR.

E. Signal Transduction

We know that ERα serine residue 118 (S118) appears to be an important site that can be phosphorylated by activated MAPK, resulting in ligand-independent ER activity (Kato, Endoh et al. 1995), and also that MAPK through its downstream effector RSK can phosphorylate ERα S167 (Joel, Smith et al. 1998a). But estrogen can lead to phosphorylation of S118 by a mechanism that does not involve MAPK in some breast cancer cells (Joel, Traish et al. 1998), and S118 can also be phosphorylated by other kinases (Chen, Riedl et al. 2000), so that the exact consequences of ER activation at this phosphorylation site is yet to be defined. Furthermore, one must keep in mind that not only does phosphorylation by MAPK activate ER, but estrogen reciprocally also influences activation of MAPK [reviewed in (Driggers and Segars 2002)], suggesting the existence of feedback loops between the two signaling systems. Thus the importance of ER phosphorylation may be dependent not only on MAPK, but also on other signaling molecules that impact on MAPK, which is the subject of Aim 1 of this proposal.

ER can also be phosphorylated and activated by a number of other pathways, including the PI3K/AKT pathway (Martin, Franke et al. 2000), a mechanism that has been implicated in TR (Campbell, Bhat-Nakshatri et al. 2001), as well as Protein Kinase A signaling which can increase the estradiol-like activity of Tam (Fujimoto and Katzenellenbogen 1994), and Protein Kinase C (Cho and Katzenellenbogen 1993). Interestingly, there are also multiple feedback systems between ER and these other intracellular signaling kinases, suggesting that these feedback loops may be integrated, a concept which could help explain the heterogeneity, cell type, and tissue specific nature of ER's responses. The role of growth factors and phosphorylation in estrogen signaling is obviously a much studied area, and represents a scientific endeavor that is now bearing fruit with the recent introduction of numerous signal transduction inhibitors into clinical practice (Johnston, Head et al. 2003).

F. Membrane Initiated Steroid Signaling

In addition to the classical effects of ERα acting as a transcription factor, it has been demonstrated that estrogen can exert early membrane signaling events that do not require classical genomic transcription, although these effects, termed membrane initiated steroid signaling, or MISS can overlap with and potentially synergize with classical transcriptional mechanisms, thus complicating the dissection of MISS in ultimate hormone action. There is growing supportive evidence for the existence of a plasma membrane receptor localized mainly in caveolae (Pappas, Gametchu et al. 1995; Chambliss, Yuhanna et al. 2000; Razandi, Pedram et al. 2000; Razandi, Pedram et al. 2003). Membrane-bound ERα has been shown to stimulate the release of heparin-binding epidermal growth factor which subsequently activates the EGFR (Prenzel, Zwick et al. 2000). Thus, MISS can activate proliferation through the EGFR/ras/MAPK signaling cascade, and conversely inhibit apoptosis via negative regulation of JNK-dependent mechanisms, possibly through bcl-2 (Razandi, Pedram et al. 2000). There are reports that the membrane form of ERα represents a 46 kDa variant form of the receptor (Li, Haynes et al. 2003), however, clinical evidence for a role of this form in breast cancer is currently lacking. MISS can also activate other signaling cascades, such as the IGF-1 pathway, P13 kinase, and G protein coupled receptors (Razandi, Pedram et al. 1999) (Kahlert, Nuedling et al. 2000) (Song, Santen et al. 2002). How MISS balances with the nuclear functions of ERα, and its role if any, in HR are an open area of research. However, an understanding of the molecular mechanisms associated with MISS may introduce new possibilities for targeted therapies to augment current hormonal therapies.

G. Progesterone Receptors (PRs) A AND B

The two PR isoforms, PR-A and PR-B, possess different in vitro and in vivo activities, suggesting that in tumors the ratio of their expression may control hormone responsiveness. In general, PR-B are strong transcriptional activators while PR-A can act as dominant repressors of PR-B and ER. Thus their balance may affect tamoxifen response in breast cancers. Expression of the two isoforms correlated with each other, as well as with ER. Further analyses revealed that patients with PR-positive tumors but high PR-A/PR-B ratio, which were often caused by high PR-A levels, were 2.76 times more likely to relapse than patients with lower ratios, indicating resistance to tamoxifen. In breast cancers, total PR (as measured by ligand binding assay) has many of the same prognostic and predictive implications as ER (Ravdin, Green et al. 1992; Fisher, Perera et al. 1998). Approximately half of primary breast tumors are positive for both PR and ER, whereas less than 5% are negative for ER but still positive for PR. In addition, well-differentiated tumors are more likely to be PR-positive than are poorly differentiated tumors. Several clinical studies have confirmed that elevated total PR levels correlate with an increased probability of response to tamoxifen, longer time to treatment failure, and longer overall survival (Clark, McGuire et al. 1983; Gelbfish, Davidson et al. 1988; Stonelake, Baker et al. 1994).

Furthermore, study also indicates that the high PR-A/PR-B ratios, at least in this study population, were frequently caused by excess PR-A, rather than low PR-B. Predominance of PR-A could cause tamoxifen resistance by directly repressing the transcriptional activity of ER as suggested by several in vitro studies (Vegeto, Shahbaz et al. 1993; McDonnell, Shahbaz et al. 1994; Wen, Xu et al. 1994; Kraus, Weis et al. 1995), or indirectly by PR-A directed up-regulation of genes known to be involved in tumor aggressiveness or prognosis. These data support other work indicating that PR-A rich tumors have heightened aggressiveness, and that abnormal PR-A excess is found in the healthy breasts of women with BRCA1/2 mutations (Mote, Leary et al. 2004).

Second, are the recent provocative results from the ATAC study showing only a modest advantage for anastrazole compared to tamoxifen in the ER+/PR+ group, while there was a major benefit for anastrazole in the ER+/PR− subgroup (The_ATAC_Trialists'_Group 2002; Dowsett 2003). Although this study is undoubtedly preliminary and awaits confirmation, it did involve thousands of patients, and supports the data from Bardou and colleagues (Bardou, Arpino et al. 2003). Finally, in view of recent trials showing a significant advantage for the sequence of tamoxifen followed by an aromatase inhibitor, it is an intriguing possibility that PR status could be used to select initial therapy. ER and PR-positive tumors might best be treated by tamoxifen followed by an aromatase inhibitor, while ER+/PR— tumors might receive initial treatment with an aromatase inhibitor because of their relative resistance to tamoxifen. This hypothesis should be tested in ongoing clinical trials.

VIII. Additional Breast Cancer Therapies

In an embodiment of the invention, a chemotherapy-resistant tumor, such as a tamoxifen-resistant tumor, is predicted/diagnosed in individuals with breast cancer using methods described herein. For those individuals wherein tamoxifen resistance occurs and is no longer effective, it is desirable to employ an additional/alternative regimen for treatment of their breast cancer. This regimen may utilize chemotherapy, radiation, surgery, immunotherapy, hormone therapy, gene therapy, and so forth, and combinations thereof.

A. Chemotherapy

Examples of chemotherapeutic agents that may be employed upon development of resistance to tamoxifen include Letrozole, cyclophosphamide, pamidronate, doxyrubicin, doxyrubicin/adriamycin, capecitabine and/or docetaxel, 5′ fluorouracil, arimidex, fulvestrant, cyclophosphamide, raloxifene, gefitinib, trastuzumab, petuzumab, herceptin, and/or paclitaxel. In addition to these exemplary chemotherapeutic agents, any analog or derivative variant thereof are within the scope of the invention.

B. Radiotherapy

Radation-based therapies are useful in conjunction with tumors resistant to therapy. That is, other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell may bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered to those individuals have tamoxifen-resistant tumors. Delivery of a vector comprising a polynucleotide encoding a cancer-treating activity will have an anti-hyperproliferative effect on target tissues. A variety of proteins are encompassed within the invention, exemplary embodiments of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p161NK4 has been biochemically characterized as a protein that specifically binds to and inhibits CDK4, and thus may regulate Rb phosphorylation (Serrano et al., 1993; Serrano et al., 1995). Since the p161NK4 protein is a CDK4 inhibitor (Serrano, 1993), deletion of this gene may increase the activity of CDK4, resulting in hyperphosphorylation of the Rb protein. p16 also is known to regulate the function of CDK6.

p161NK4 belongs to a newly described class of CDK-inhibitory proteins that also includes p16B, p19, p21Waf1/Cip1, and p27KIP1. The p161NK4 gene maps to a chromosome region frequently deleted in many tumor types. Homozygous deletions and mutations of the p161NK4 gene are frequent in human tumor cell lines. This evidence suggests that the p161NK4 gene is a tumor suppressor gene. This interpretation has been challenged, however, by the observation that the frequency of the p161NK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p161NK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, Bik/p27 fusions, anti-thrombotic genes (e.g., COX-1, TFPI), PGS, Dp, E2F, ras, myc, neu, raf, erb, fins, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl 2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl 2 cell death regulatory proteins which share in common structural and sequence homologies. These different family members have been shown to either possess similar functions to Bcl 2 (e.g., BclXL, BclW, BclS, Mcl-1, A1, Bfl-1) or counteract Bcl 2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

IX. DUSP6, EBP50, and/or RhoGDIa in Breast Cancer and Resistance

In certain embodiments of the invention, an expression profile indicative of resistance to cancer therapy comprises one or more of DUSP6, EBP50, or RhoGDIa.

DUSP6 is a dual-specific phosphatase that dephosphorylates both pSer/Thr and pTyr. It is known to be specific for ERK1 and ERK2, but it does not dephosphorylate JNK or p38. It has been demonstrated to be a potential tumor suppressor in pancreatic tumor cells. The present invention demonstrates that DUSP6 (dual specificity phosphatase 6) (which at least may also be referred to as MAP kinase phosphatase 3 (MKP3), serine/threonine specific protein phosphatase, HGNC:3072, or PYST1) is involved in breast cancer, such as being involved in breast cancer resistance, and in specific embodiments DUSP6 is utilized at least in part as a marker of resistance. In specific embodiments, overexpression of DUSP6 is associated with breast cancer resistance (see Examples herein).

Although the expression level of DUSP6 is useful as a marker of breast cancer resistance, the expression level may be ascertained by any suitable methods in the art. For example, the expression level of DUSP6 may be determined as one of a plurality of polynucleotides being monitored for expression, such as with the methods of the present invention, or the expression level of DUSP6 may be identified alone. In specific embodiments, the expression levels of DUSP6 are identified based on RNA levels, based on corresponding protein levels, or both. The expression level of DUSP6 may be identified in a microarray, northern blot, western blot, quantitative RT-PCR, and so forth.

Additionally, or alternatively, the expression level of EBP50 and/or RhoGDIA may be ascertained by any suitable methods in the art, and the level may be determined alone or with a plurality of polynucleotides being monitored for expression. In specific embodiments, the expression levels of EBP50 and/or RhoGDIa are identified based on RNA levels, based on corresponding protein levels, or both. The expression level of EBP50 and/or RhoGDIa may be identified in a microarray, northern blot, western blot, RT-PCR, and so forth.

### EXAMPLES

The following examples are offered by way of example and are not intended to limit the scope of the invention in any manner.

### Example 1

### Exemplary Materials and Methods

Reagents, Hormones, and Antibodies

17β-Estradiol (E2) and 4-hydroxy-tamoxifen (4-OH-Tam) were from Sigma (St. Louis, Mo.). ICI 182,780 was obtained from Astrazeneca (Macclesfield, UK). The MEK1,2 inhibitor PD98065 was from Calbiochem (La Jolla, Calif.). p-nitrophenyl phosphate (PNPP) and pNPP assay buffer were obtained from Upstate Biotechnology (Charlottesville, Va.). Antibodies used for immunoblotting were to; phospho-MAPK (Thr202/Tyr204), phospho-Ser118-ERα, and total MAPK p42/44 (Cell Signaling Technology, Beverly, Mass.); ERα (6F-11, Vector Labs Inc., Newcastle, UK); AIB1, p190 (BD Transduction Lab, Los Angeles, Calif.); ERK2 (C-14), PR(C-19, and H190), RhoGDI (Santa Cruz, Santa Cruz, Calif.); Cyclin D1 (Upstate Biotechnology); V5 (InVitrogen, Carlsbad, Calif.); HA (Covance, Berkeley, Calif.).

Tumor Specimens, Expression Microarray Analysis, and Semiquantitative RT-PCR

A cohort of frozen breast tumor specimens from nine patients who received adjuvant Tam was selected from the tumor bank of The Breast Center, Baylor College of Medicine, for use in the RNA analyses. This study was approved by the Baylor College of Medicine Institutional Review Board in accordance with federal human research study guidelines. Within this cohort, metastatic tumors from five patients who developed their recurrent lesion within 1-11 months while undergoing Tam treatment (Tam-resistant), and four primary tumors that were collected at the time of initial diagnosis from patients who remained disease-free with a median follow-up of 106 months (Tam-sensitive) were included.

Total cellular RNA was extracted from 100 mg of pulverized tumor powder using Trizol reagent (InVitrogen) followed by Qiagen RNeasy column purification (Qiagen, Valencia, Calif.). Double stranded cDNA synthesis, combined in vitro transcription (Enzo Biochem, New York, N.Y.), and biotin labeling was carried out in accordance with protocols recommended by Affymetrix (Santa Clara, Calif.). For each tumor specimen, 15 μg of cRNA was hybridized onto Affymetrix HGU95A GeneChips using recommended procedures for hybridization, washing, and staining with streptavidin-phycoerythirin.

The GeneChips were scanned, and feature quantitation was performed using MAS5.0 (Affymetrix). Data were normalized using mean intensity, and modeled to estimate expression using dChip analysis with the perfect match-only modeling algorithm (15); class comparisons were performed using BRB Array Tools developed by Richard Simon and Amy Peng and available on the World Wide Web, and t-tests were performed with randomized variance modeling. MKP3 expression data for Tam sensitive and resistant tumors are displayed using a box-and-whisker plot, and compared using a Wilcoxon rank sum test.

Semi-quantitative RT-PCR (sqRT-PCR) was performed of the tumor total RNAs subjected first to DNase treatment prior to cDNA synthesis and amplification. Briefly, primers to MKP3 and the GAPDH control gene were utilized. Duplicate reactions were prepared and samples were taken at alternating cycle numbers between 20 and 28 cycles to ensure that the sqRT-PCR reaction products were in a linear range of amplification. These samples were then diluted with μl loading buffere, and μl of each sample was loaded onto % acrylamide gels. After electrophoresis at 20V for 18 h, gels were fixed, transferred to filter paper, and dried. The absolute intensities of single bands for MKP3 and GAPDH were analyzed using phosphorimager quantification (Bio-Rad Laboratories, Hercules, Calif.). sqRT-PCR PCR product band intensities were then quantitatively compared with normalized, model-based estimates of expression from the GeneChip data.

Cells and Stable Transfection

MCF-7 cells were originally obtained from Dr. Benita Katzenellebogen, but have been maintained in the inventors' laboratory for over 10 years (Fuqua et al., 2000). MCF-7 cells were maintained in Minimal Essential Medium (MEM, InVitrogen) supplemented with 5% fetal bovine serum (Summit Biotechnology, Fort Collins, Colo.), 200 U/ml penicillin, and 200 μg/ml streptomycin. Cells were incubated at 37° C. in 5% CO₂. To generate MCF-7 cells stably overexpressing MKP3, 5 μg of the plasmid pcDNA3-MKP3-V5 (Furukawa et al., 2003) or empty vector (pcDNA3-His-V5, InVitrogen), were transfected into the cells using Fugene 6 reagent Roche Clinical Laboratories, Indianapolis, Ind.) in 100 mm tissue culture dishes following the manufacturer's protocol. Stable clones overexpressing MKP3 were selected as described (Fuqua et al., 2000), and positive clones were identified using immunoblot analysis with an anti-V5 antibody (1:5000 dilution)

Cell Extracts and Immunoblots

Cells grown in 100 mm dishes were starved in phenol red-free (PRF), serum-free MEM (Specialty Media, Phillipsburg, N.J.) for 48 hours, and were then treated for 2 hours with vehicle (ethanol), estrogen (100 nM, 20), or OHT (100 nM). After treatment, cells were rinsed twice with ice-cold phosphate-buffered saline (PBS) and were then lysed immediately with 200 μl of cell lysis buffer (20 mM Tris HCl, pH 7.4, 150 mM NaCl, 1 mM B-glycerophosphate, 1 mM sodium orthovanadate, and 10% glycerol plus 1:100 proteinase inhibitor cocktail III) (Calbiochem, LaJolla, Calif.) per 100 mm tissue culture. The cell lysates were cleared by centrifugation at 16,100×g for 10 minutes at 4° C. Protein concentration was determined with the BCA Protein Assay kit (Pierce, Rockfold, Ill.) according to the manufacturer's directions. Equal amount of cell extracts were resolved under denaturing conditions by electrophoresis in 8-10% polyacrylamide gels containing sodium dodecyl sulfate (SDS-PAGE), and transferred to nitrocellulose membranes by electroblotting (Schleicher & Schuell, Keene, NH). After blocking of the transferred nitrocellulose membrane with TBST (20 mM Tris pH 7.6, 150 mM NaCl, 0.1% Tween-20) supplemented with 5% non-fat milk for 1 hour at room temperature, the membrane was incubated with primary antibodies either for 1 hour at room temperature (anti-ERα, 1:100 dilution; anti-AIB1, 1:100 dilution; anti-ERK2, 1:500 dilution; anti-Cyclin D1,1:500 dilution; anti-PR, C-19, 1:200 dilution; anti-p190, 1:1000 dilution; anti-V5, 1:5000 dilution), or overnight at 4° C. (anti-phospho MAPK, 1:1000 dilution; anti-phosphor-Ser118-ERα; 1:1000 dilution; MAPK p42/44, 1:1000 dilution), incubated with secondary antibodies for 1 hour at room temperature and then developed with Enhanced Chemiluminescence Reagents (Amersham Pharmacia Biotech, Piscataway, N.J.).

Transient Transfection and Ubiquination Assays

Cells were staved in PRF MEM for 24 hours, and then 2×10⁶ cells were seeded into 100 mm tissue culture plates, and incubated for another 24 hours. Cells were transiently transfected with Fugene 6 reagent (Roche, Indianapolis, Ind.) following the manufacturer's protocol. Each plate was transfected with 1 μg pcDNA 3.1-PR-B (subcloned by BamHI digestion from pSG5-human PR-B clone (Richer et al., 1998) obtained from Dr. Kathryn Horwitz, University of Colorado Health Sciences Center, 1 μg MKP3-V5, and 3 μg pcDNA-HAUbiquitin (kindly provided by Dr. Xinhua Feng, Baylor College of Medicine). Twenty-four hours later, cells were switched to serum free, PRF MEM, incubated for another 24 hours. In some experiments were pretreated with the 50 μM MG-132 (Calbiochem, LaJolla, Calif.) for 2 hours, and then treated with vehicle (ethanol), estradiol (E2, 100 nM), or 4-OH-Tam (100 nM) for an additional 2 hours. Cells were then harvested in 1 ml cell lysis buffer supplemented with 50 μM MG-132. Cell lysates from each treatment were precleared with the addition of 50 μl 50% protein A slurry (Amersham), and immunoprecipitated with the addition of anti-PR antibodies C-19 and H190 (2 μg each), and 20 μl 50% protein A slurry. The pellets were collected by centrifugation, washed 3 times with 1 ml cell lysis buffer, solubilized by boiling in sodium dodecyl sulfate (SDS) loading buffer, and the proteins separated resolved under denaturing conditions by electrophoresis in 8-10% polyacrylamide gels containing SDS (SDS-PAGE), and transferred to nitrocellulose membranes (Schleicher & Schuell) by electroblotting. After blocking of the transferred nitrocellulose membrane with TBST supplemented with 5% non-fat milk for 1 hour at room temperature, the membrane was incubated with anti-HA antibody (anti-HA11, 1:1000 dilution) for 1 hour at room temperature, incubated with secondary antibody for 1 hour at room temperature, and developed with Enhanced Chemiluminescence Reagents (Amersham).

Immunoprecipitation and Phosphatase Assays

MKP3-overexpressing cells and control vector transfected cells were treated as described above with ethanol, E2, or 4-OH-Tam, and lysed in buffer B containing 20 mM Tris-HCl (pH 7.0), 150 mM NaCl, 1% Triton X-100, 0.25 M sucrose, 1 mM EDTA, 1 mM EGTA, 0.1% β-mercaptoethanol, 1 mM PMSF, and 1 μg/ml leupeptin. The cell lysates were cleared and the protein concentrations were determined as detailed above. 300 μg of cell lysate at 1 μg/μl was immunoprecipitated by the addition of 1 μg anti-V5 antiserum, and 20 μl 50% protein G slurry. After three times washing of the pellets with 1 ml buffer B, a phosphatase reaction (Kim et al., 2003) was initiated by the addition of 50 mM pNPP in 80111 of 50 mM Tris-HCl (pH 7.0), and incubated at 30° C. for 1 hour. The reactions were then quenched by the addition of 20 μl 5N NaOH, and pNPP hydrolysis was measured at 405 nm. The nonenzymatic hydrolysis of the substrate was corrected by measuring the control vector transfected immunoprecipitates. The amount of product p-nitrophenol was determined from the absorbance at 405 nm. The phosphatase assays were performed in triplicate, n=3 separate experiments. To detect MKP3 protein in the extracts, duplicate immunoprecipitates were resolved by SDS-PAGE, transferred to nitrocellulose, and immunobloted with anti-ERK2 antibody. After exposure, the same nitrocellulose filter was then stripped in the stripping solution (Pierce), and reimmunoblotted with anti-V5 antibody.

Xenograft Studies

MCF-7 vector control and MKP3-transfected cells were established as xenografts in ovariectomized 5- to 6-week-old BALB/c athymic nude mice (Harlan Sprague Dawley, Madison, Wis.) supplemented with 0.25-mg 21-day-release E2 pellets (Innovative Research, Sarasota, Fla.) by inoculating the mice subcutaneously with 5×10⁶ cells, as described previously (Osborne et al., 1995). When tumors reached ˜250 mm³ (i.e., in 21 days) animals were randomly allocated to continue E2 (n=6 per group), or to estrogen withdrawal plus tamoxifen citrate (+Tam, n=6 per group; 500 μg/animal given subcutaneously in peanut oil, 5 days/week) for another 30 days. Tumor growth was assessed and tumor volumes were measured as described previously (Osborne et al., 1995). Mice were anesthetized with isoflurane before tumor removal; tumor tissues were kept at −190 0 C for later analyses. Animal care was in accordance with institutional guidelines.

Anchorage-Independent Growth Assays

Cells were starved for 2 days in PRF MEM (InVitrogen) supplemented with 5% fetal bovine serum (FBS, Summit Biotechnology, Fort Collins, Colo.), 6 ng/ml insulin, 200 units/ml penicillin, and 200 μg/ml streptomycin. Soft agar assays were performed in sixwell plates. Into each well, 1.5 ml of prewarmed (50° C.) 0.7% SeaPlaque agarose (FMC, Rockland, Me.) was added and dissolved in PRF MEM supplemented with 5% charcoal stripped FBS (Summit, Fort Collins, Colo.) to serve as the bottom layer, and allowed to solidify at 4° C. until to use. 0.5×10⁴ cells as a single cell suspension were suspended in prewarmed (37° C.) of the same media, 4 ml of 0.5% SeaPlaque agarose was then added, and this suspension then plated as the top layer by adding it dropwise to the solidified bottom layer plates. Plates were cooled for 2 hours, and then transferred to a 37° C. humidified incubator. Two days after plating, media containing control vehicle, 1 nM estradiol, 100 nM 4-OH-Tam, 100 nM OHT plus 10 nM ICI, or 100 nM OHT plus 20 nM PD98065 was added to the top cell layer, and the appropriate media was replaced every two days. After 14 days, the colonies were fixed, and the colony number of colonies >50 cells from quadruplicate assays were then counted. The data shown is the mean colony number of four plates, and is representative of two independent experiments.

### Example 2

### MKP3 Overexpression and TR

To identify genes whose expression is associated with the development of TR, compared primary tumors with metastatic tumors that arose during adjuvant Tam treatment using expression microarray and sqRT-PCR analyses. The approach and tumor selection criteria differed from that recently reported by Ma et al. (2004), who compared primary tumors of patients that were disease-free after adjuvant Tam treatment to primary tumors of patients that recurred with a median time to recurrence of 4 years to generate a gene expression profile to predict clinical outcome (Ma et al., 2003). In specific embodiments, it is more likely to identify genes whose differential expression reflected acquired TR with this selection criteria, and whose function might itself be affected by Tam treatment.

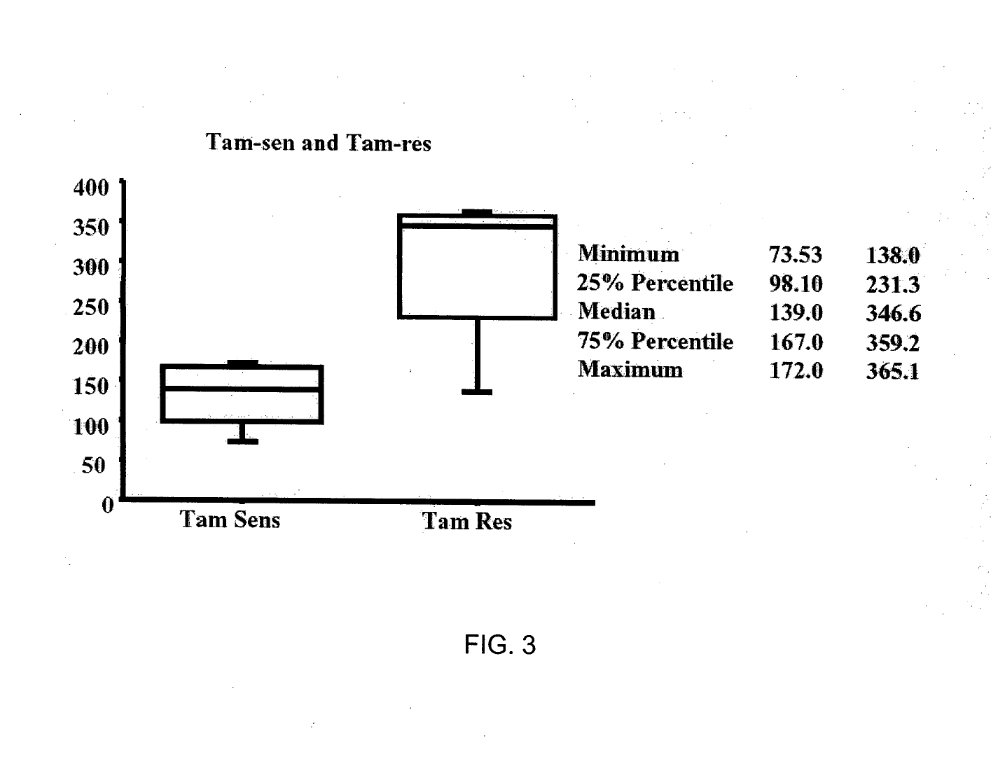

Gene expression analysis of the cohort identified MKP3 as being more highly expressed in the Tam-resistant tumor group as compared to the Tam-sensitive tumor group (p<0.007, FIG. 3). The data is shown in the form of a whisker box plot where the minimum, maximum, 25th, and 75th percentiles are presented. To confirm the measurement of MKP3 RNA levels, expression values derived from the Affymetrix data were correlated with values obtained from sqRT-PCR of RNA from individual tumors (Table 1 elsewhere herein).

Differentially expressed genes were identified using two sample t-test and classifiers predicting resistance were determined at either the p=0.01 level of significance (Table 1) or p=0.05 level of significance (Table 2). These genes showed 1.2-5.3-fold decreases or 1.2-2.8-fold increases in geometric mean gene expression in resistant compared to sensitive tumors.

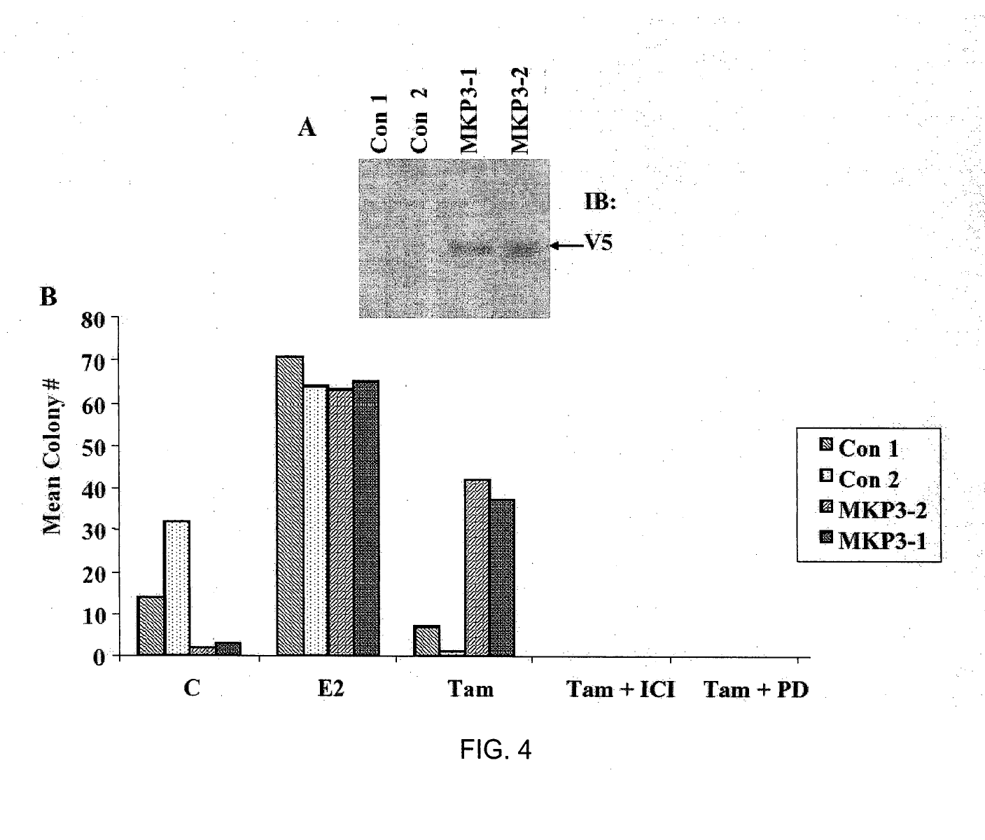

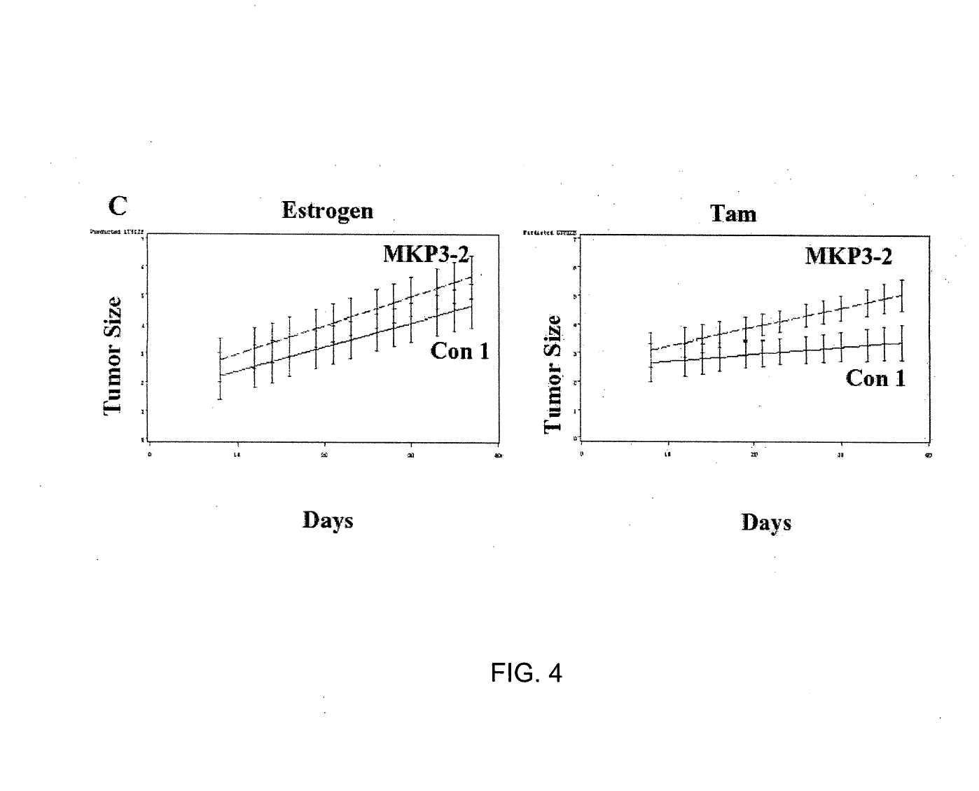

To generate a model of TR, ERα-positive MCF-7 cells were stably transfected with either empty vector plasmid as a control, or a plasmid encoding MKP3. Cells were drug selected for plasmid expression, cloned by limiting dilution, and several resulting clones were screened for expression of MKP3 using an anti-V5 antibody to the V5 tag introduced at the carboy-terminus of MKP3 (Fuqua et al., 2003). An immunoblot analysis of two MCF-7-control clones (FIG. 4A, Con 1 and 2) and two MCF-7-MKP3-overexpressing clones (FIG. 4A, MKP3-1 and 2) demonstrates ectopic expression of MKP3 in the stable transfectants. Subsequent stripping and reprobing of the membrane with P190 antibodies verified equal sample loading.

In pancreatic cells, ectopic expression of MKP3 is reported to result in a suppression of cell growth (Fuqua et al., 2003). Thus, the growth characteristics of the MCF-7 stable MKP3 transfectants under different hormonal conditions were investigated using anchorageindependent soft agar assay (FIG. 4B). As expected, MCF-7 vector control cells exhibited low colony formation in the absence of E2 (C1 and C2, C treatment); treatment with E2 increased the number of colonies, and Tam treatment reduced the number of colonies of control cells. Ectopic expression of MKP3 reduced the number of colonies formed in soft agar in estrogen-deprived conditions (MKP3-1 and MKP3-2, C), however the estrogen-induced colony formation of the MCF-7-MKP3 clones was equivalent to those obtained with vector control cells. In contrast, the number of soft agar colonies formed in the presence of Tam was increased ˜20-fold in the two MKP3-overexpressing transfectants compared to vector control cells (P<0.05). Both the steroidal ERα antagonist ICI182,780, and the MEK1,2 inhibitor PD98065 completely blocked Tam-induced colony formation in vector control and MKP3-transfected cells. These results indicate that whereas the basal growth of MCF-7-MKP3 cells may be negatively affected concomitant with MKP3 overexpression, Tam treatment might either relieve this growth suppression or act as an agonist to increase the colony forming efficiency of MKP3-overexpressing breast cancer cells.

To analyze the endocrine sensitivity of the MKP3-overexpressing cells in more detail in vivo, the ability of MCF-7-vector control 1 and MCF-7-MKP3-2 transfectants to form tumors in athymic mice was examined. Xenografts were established in ovariectomized mice supplemented with estrogen for 21 days. Mice were then randomized to continue estrogen treatment, or estrogen withdrawal plus Tam, and tumor growth was monitored over time (FIG. 4C). The main questions that were addressed were whether MKP3-expressing tumors grew differently in the presence of estrogen, and whether they responded similarly to Tam treatment. This was examined by fitting the data in an exponential growth model, and testing whether growth rates were different between the groups; the analyses were done separately for estrogen-treated (FIG. 4C, left panel) and Tam-treated animals (right panel). There was no difference in the estrogen-stimulated growth of the MCF-7-vector control and MKP3-overexpressing cells (P=0.52). However, the growth rate of MKP3-expressing cells was significantly increased in Tam-treated tumors (P=0.047). These in vivo data are thus consistent with the observed effect of Tam treatment on MKP3-overexpressing cells in the colony formation assay, and with the discovery of higher levels of MKP3 RNA in Tam-resistant metastatic breast tumors.

Cross-Talk Between MKP3, MAPK, and ERα Signaling Pathways

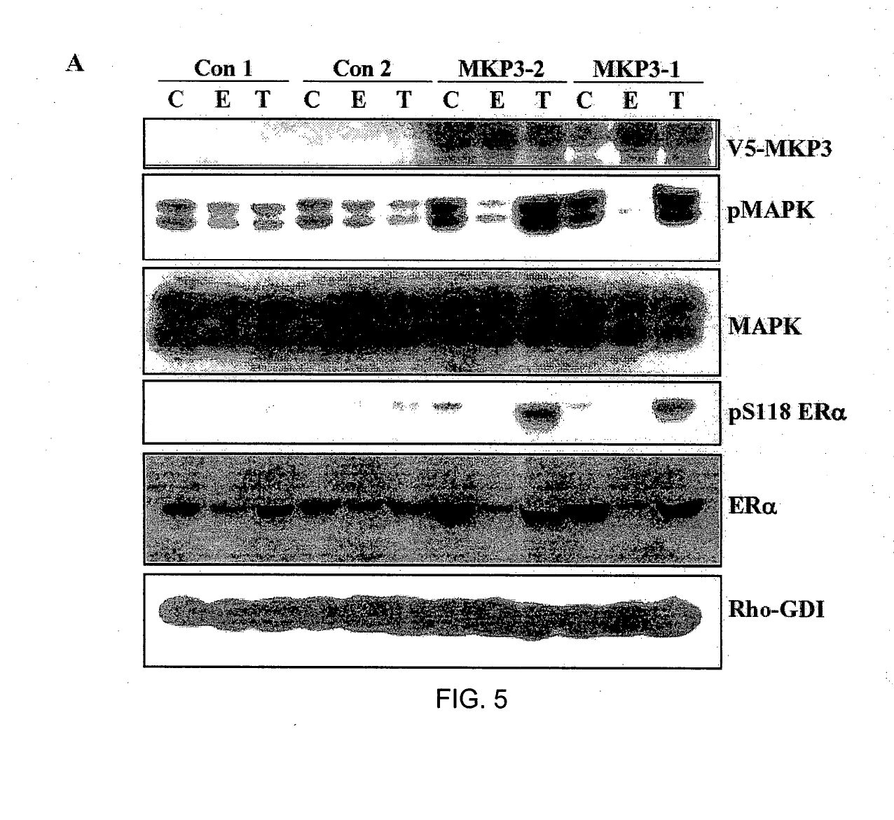

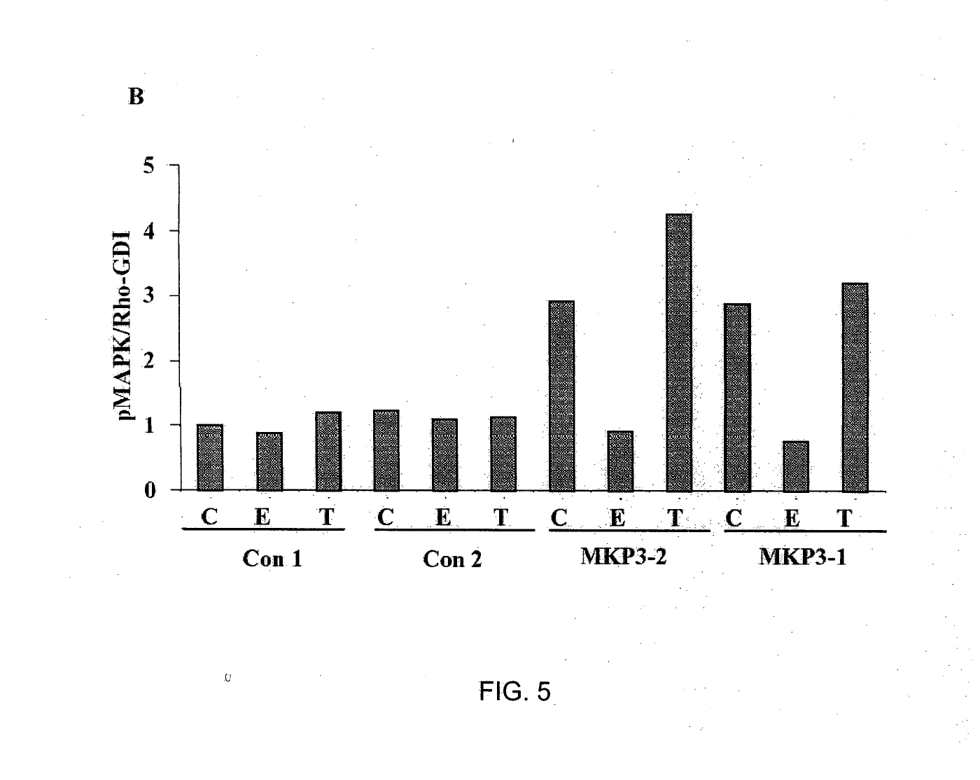

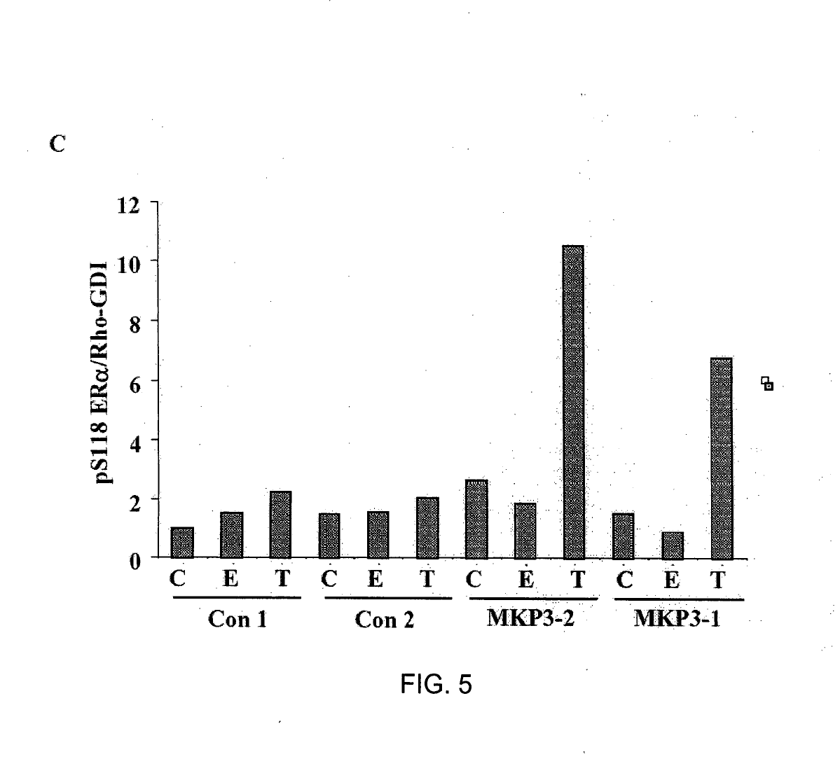

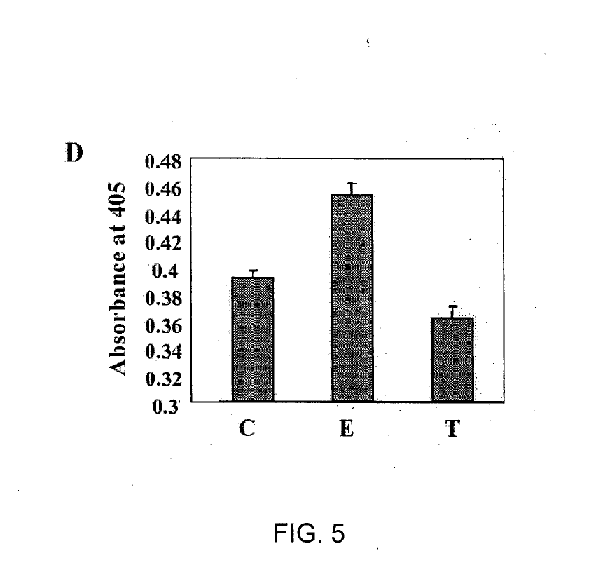

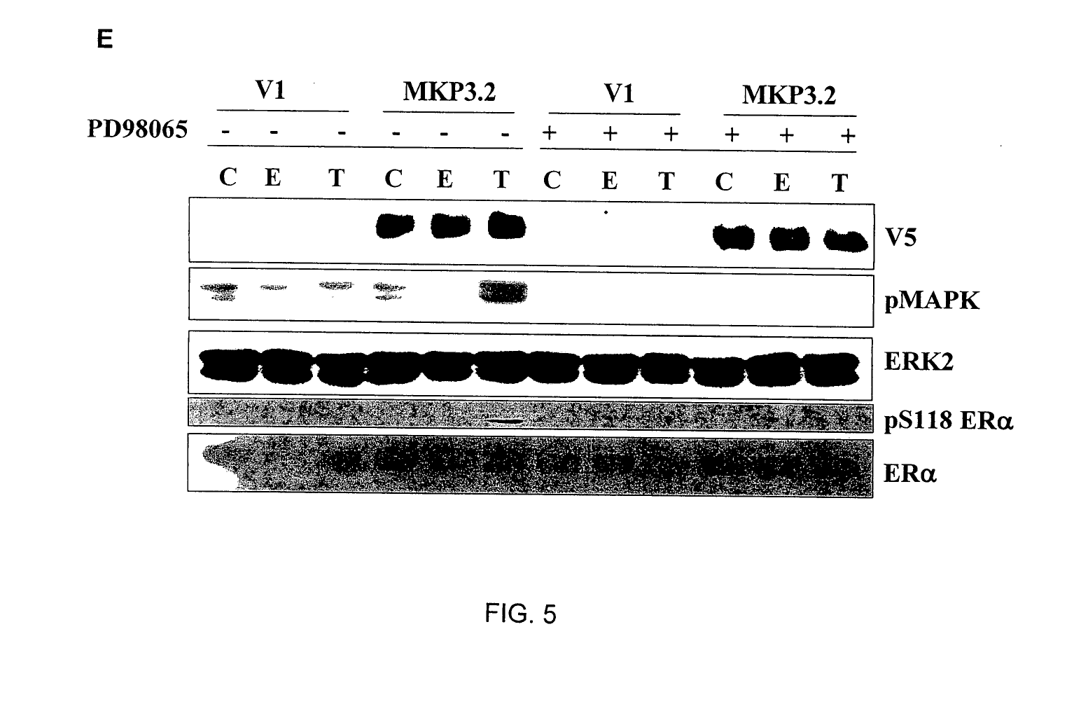

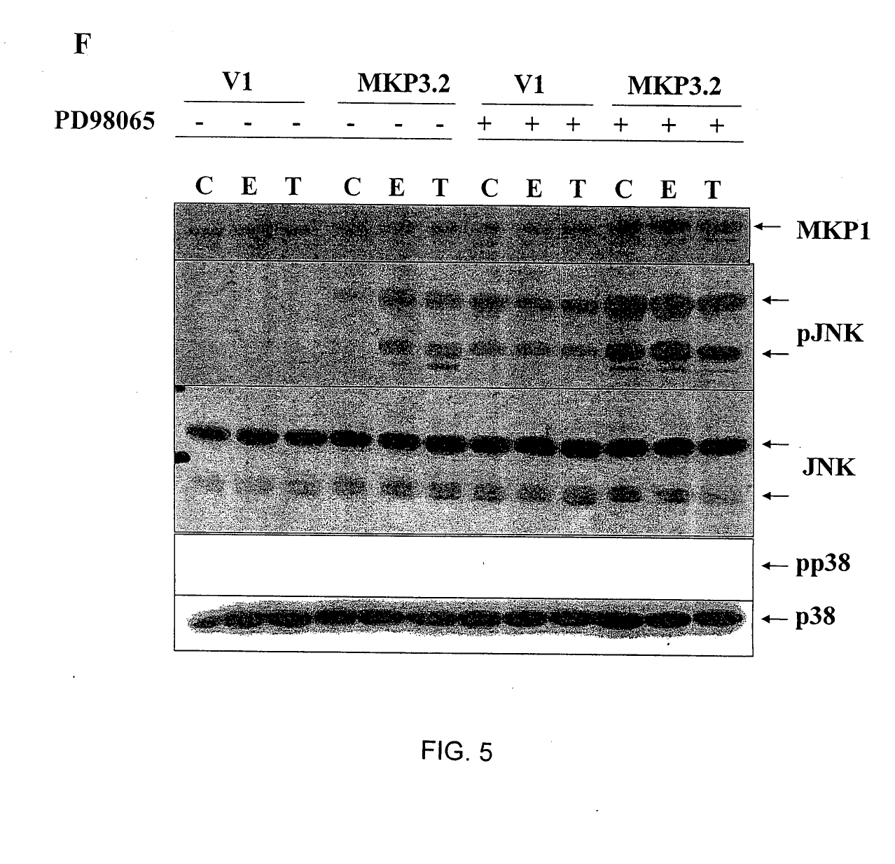

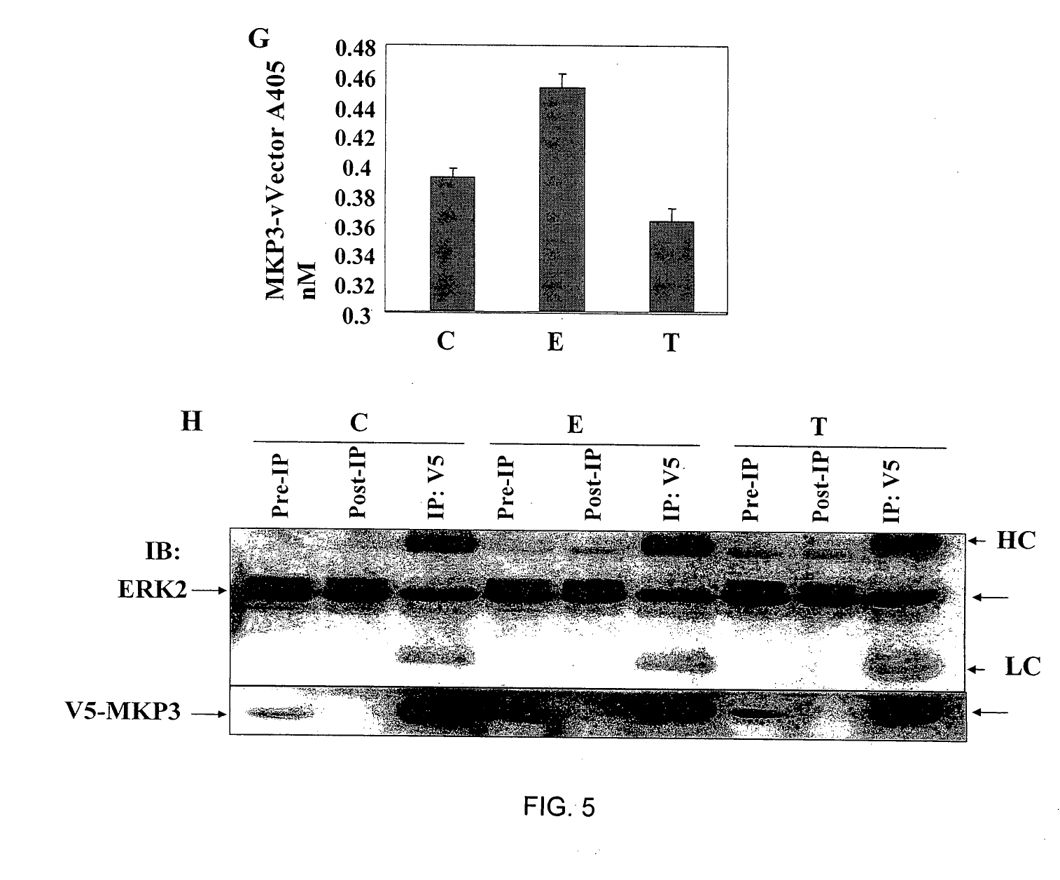

MAPK activity is tightly regulated by phosphorylation and dephosphorylation. MKP3 participates in a bidirectional regulatory loop with ERK2 MAPK, whereby ERK2 substrate binding is associated with catalytic activation of MKP3, and activated MKP3 negatively regulates pERK2 (Camps et al., 1998). Since there are conflicting reports concerning whether estrogen stimulation activates MAPK in MCF-7 breast cancer cells (Lobenhofer et al., 2000; Migliaccio et al., 1996), it was next assessed the effect of estrogen or Tam on the activation of MAPK in MCF-7 vector controls and MKP3 transfectants (FIG. 5A, Con1 and 2, and MKP3-1 and 2). Cells were maintained under estrogen-depleted conditions for 2 days, treated for 2 hours with either estrogen or Tam, and cellular extracts prepared. Immunoblot analysis with anti-V5 was used to detect ectopic MKP3 expression in the two MKP3 transfectants (FIG. 5A, top panel). FIGS. 5B and 5C provide quantitation of results in FIG. 5A.

In vector-alone transfectants, pMAPK was not induced with either short-term (2-30 minutes, data not shown), or two hours of hormonal treatment (FIG. 5). In contrast, higher levels of pMAPK were seen in the control and Tam-treated MKP3-overexpressing cells compared to that seen in the estrogen-treated cells (FIGS. 5A and 5B). These results were rather paradoxical, in that the inventors predicted to find lower levels of pMAPK in cells concomitant with MKP3 overexpression, but instead observed hormonal influences on the ability of overexpressed MKP3 to modulate pMAPK. Levels of total MAPK did not appear to be affected by MKP3 overexpression. The highest activation of MAPK was observed in the Tam-treated MKP3-overexpresing cells (graphically represented in FIG. 5B).

ERα is a downstream target of pMAPK in breast cancer cells (Kato et al., 1995). It was observed that levels of phosphorylation at ERα serine 118 (PS 118 ERα) were highly induced with Tam treatment in the MKP3 transfectants (FIGS. 5A and 5C). The question is whether this dramatic induction was coupled with changes in the levels of total ERα? It has been demonstrated that ERα undergoes estrogen-dependent down regulation via the proteasomal degradation pathway (Nawaz et al., 1999). Down regulation of ERα protein was observed in both control transfected and the MKP3 overexpressing cells in the presence of estrogen. Similarly, it has been reported that Tam stabilizes the receptor (Wijayaratne and McDonnell, 2001), and this stabilization was observed in both groups of transfectants. Thus, although ERα hormonal regulation appeared to proceed normally, higher levels of pERα were induced by Tam in the MKP3 overexpressing cells, which was not associated with higher levels of total ERα as compared to control treated cells.

The effect of the MEK1,2 inhibitor PD98059 was tested on the ability of Tam to induce phosphorylation of MAPK and ERα in MKP3 overexpressing cells (FIG. 5E). It was found that PK98065 effectively inhibited the increase in pMAPK and pS118 ERα in control and Tam-treated MKP3.2 cells. The MEK inhibitor also blocked activation of MAPK in vector control cells under all the treatment conditions. This result suggests that Tam's effects in MKP3 overexpressing cells involve the MEK-ERK MAPK signaling pathway.

As a control the levels of MKP1 were examined, which is more specific for JNK and p38, but did not see changes in the levels of MKP1 concomitant with MKP3 overexpression (FIG. 5F). Similarly, no activation of p38 or changes in total p38 in MKP3 transfectants were seen. However, there was a surprising increase in pJNK in MKP3 transfectants under all hormonal conditions. When vector control cells were treated with the MEK1,2 inhibitor, pJNK was elevated, and higher levels of activation were observed in MKP3 transfectants in the presence of the inhibitor. These results were not expected, and in a specific embodiment indicates that the MEK inhibitor affects the decreased levels or activity of another MKP that up-regulates pJNK. In specific embodiments of the invention, this indicates that treatment of breast cancer cells with MEK inhibitors will concomitantly increase JNK signaling in cells, a consequence that has therapeutic relevance in resistant disease necessitating combination therapy with signal transduction inhibitors, in particular aspects of the invention.

The observed changes in pMAPK levels in MKP3 overexpressing cells was further characterized. It was questioned whether either changes in MKP3 phosphatase activity or changes in binding between MKP3 and ERK2 might underlie the changes in pMAPK. Using the artificial substrate pNPP to measure endogenous phosphatase activity, there was an inverse relationship between measured phosphatase activity and pMAPK levels in the MKP3 transfectants. As shown in FIG. 5G, levels of phosphatase activity were highest in the estrogen-treated, lowest in Tam, and intermediate in the control-treated cells. These findings in activity were inversely related to the levels of pMAPK in MKP3 overexpressing cells (FIG. 5A). These results indicate that Tam influences the ability of MKP3 to negatively regulate MAPK, in specific embodiments. There were no observed changes in the ability of MKP3 to bind to MAPK, as measured by co-immunoprecipitation and immunoblot analysis (FIG. 5H). When the levels of MKP3-bound ERK2 were compared between the different hormonal treatments, no differences were detected (C, E, T, IP:V5 lanes). Control levels of MKP3 and ERK2 were also examined in the pre-IP and post-IP extracts to ensure that adequate pulldown of IP proteins were obtained; no differences were detected. Thus, hormonal modulation of MKP3 phosphatase activity, but not changes in the ability of MKP3 to interact with MAPK may be a determinant of MAPK activation in breast cancer cells.

### Example 3

### DUSP6 (MKP3) as a Coactivator of Estrogen Receptor

There are a large number of estrogen receptor (ER) coregulatory proteins that function as signaling intermediates between the ERs and the general transcriptional machinery (for reviews, see McKenna et al. 1999, and Horwitz et al. 1996). These coregulatory proteins are components of a complex of proteins bound to the nuclear receptors, and their presence or absence can help determine if the receptors act as a transcriptional repressor or activator. Some of the coregulatory proteins, called coactivators, possess enzymatic activity, but the precise mechanism by which coactivators enhance ER transactivation function remains to be determined. The receptor interacting motif LXXLL (called the ‘NR’ box; SEQ ID NO:167) has been identified within nearly all coactivators, and these residues are indispensable for receptor interaction with coactivators (Heery et al. 1997). It may be a relative imbalance of coregulator proteins that ultimately determine ER's function and tamoxifen resistance in individual breast cancer patients, as indicated for MKP3 overexpression in patients.

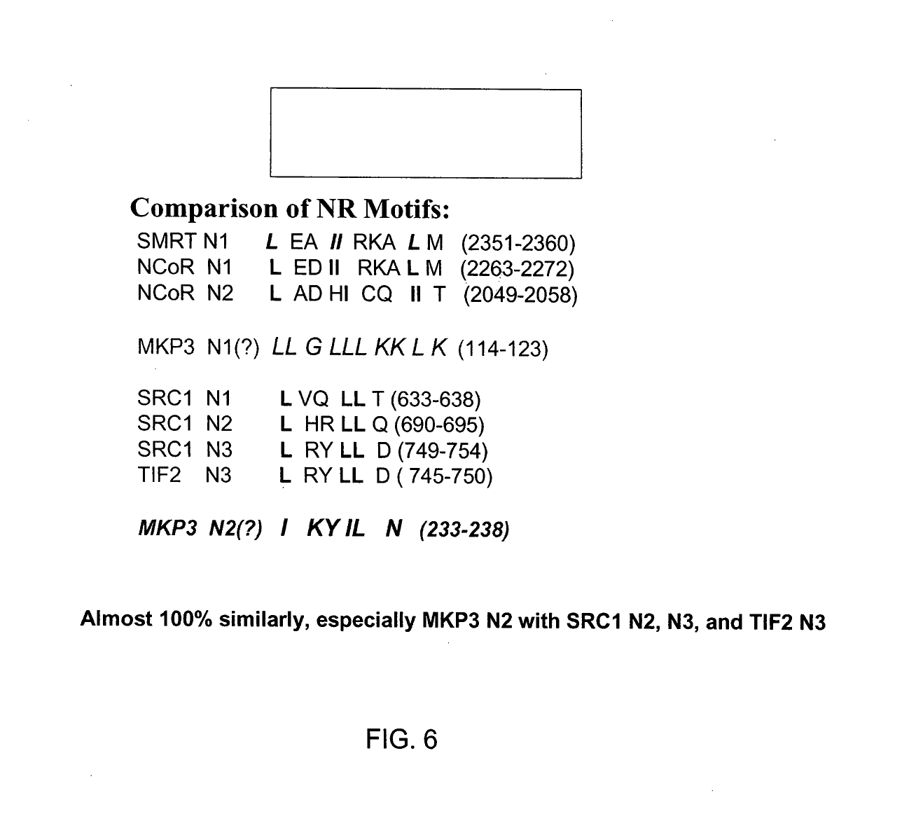

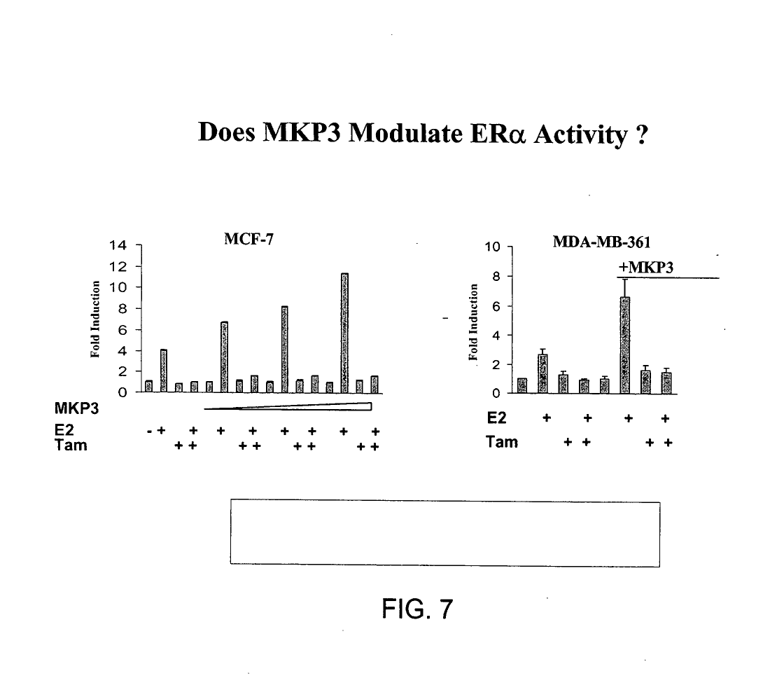

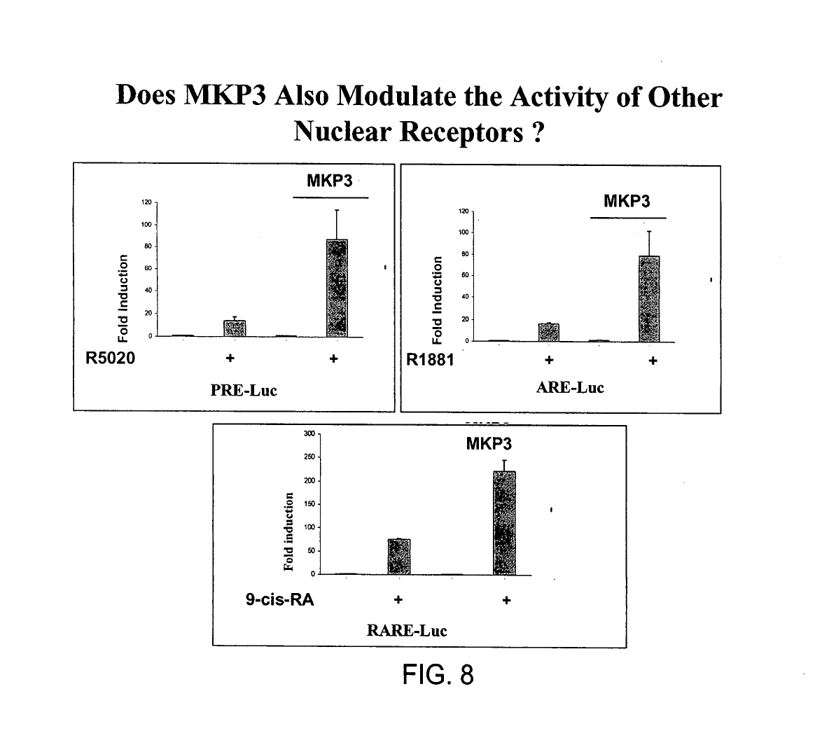

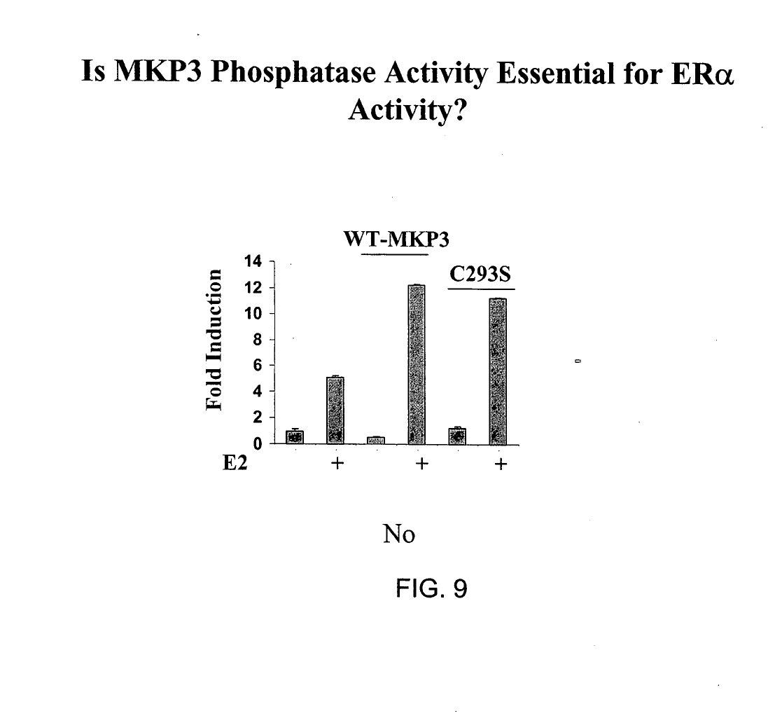

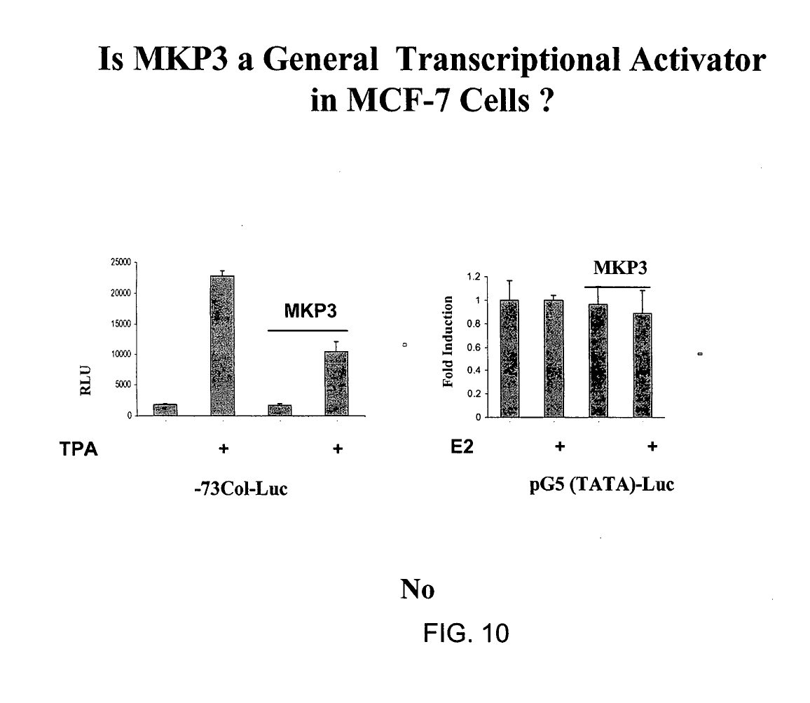

Two NR boxes were identified in MKP3 (residues #114-123, FIG. 6), indicating that MKP3 interacts with the ER and function as a receptor coactivator, in specific aspects of the invention. To demonstrate that MKP3 expression acts to increase ERα transcriptional activity (thus is a coativator), transient transactivation assays were employed using an estrogen-responsive luciferase reporter. Exogenous expression of MKP3 in two breast cancer cells lines (MCF-7 and MDA-361) increased ERα transcriptional activity (FIG. 7). MKP3 also increased progesterone receptor, androgen receptor, and retionoic acid receptor activities (FIG. 8). Thus, similar to almost all coactivators that have been discovered, MKP3 acts as a coactivator on a number of different nuclear receptors. It is also shown that MKP3 enzymatic activity is not required for it to function as a coactivator by deletion of the MKP3-residues critical for its activity (FIG. 9). MKP3 is not a general transcriptional activator, however, thus demonstrating specificity for nuclear receptors (FIG. 10). These results indicate that MKP3 in specific embodiments is mechanistically functioning directly through the ER to cause tamoxifen resistance; however, it might also be acting indirectly through its regulation of Erk1/2 MAPK, a pathway which can then affect ER function (FIGS. 5A and 5D).

### Example 4

### ER Regulated Gene Transcription

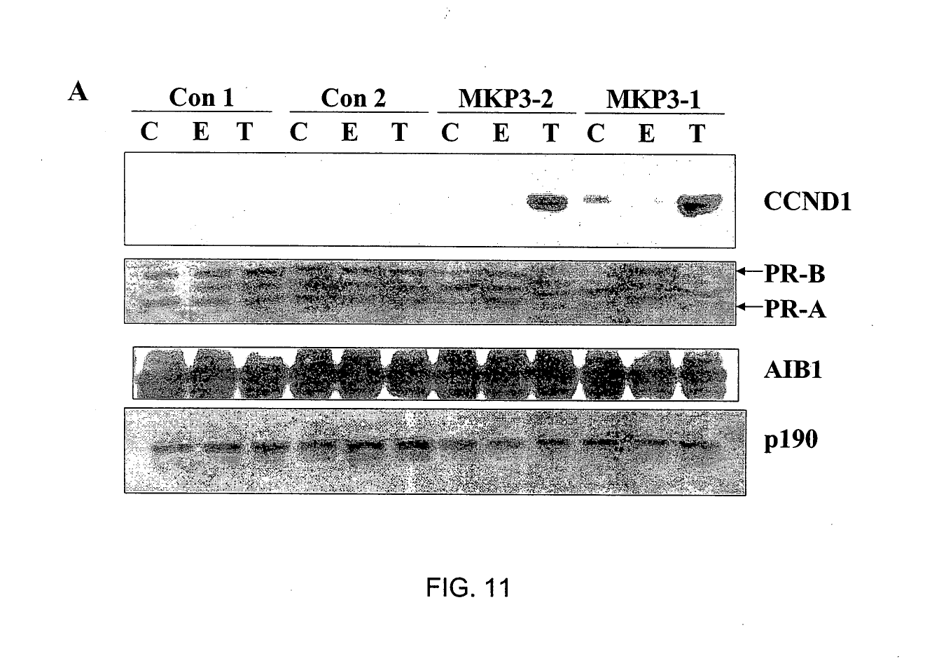

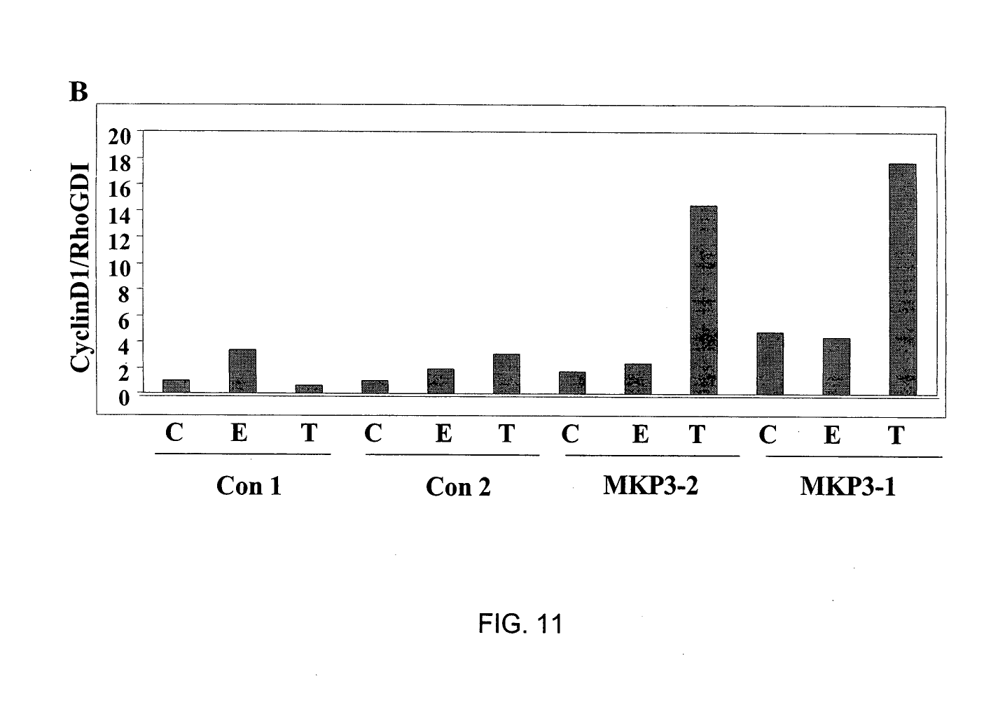

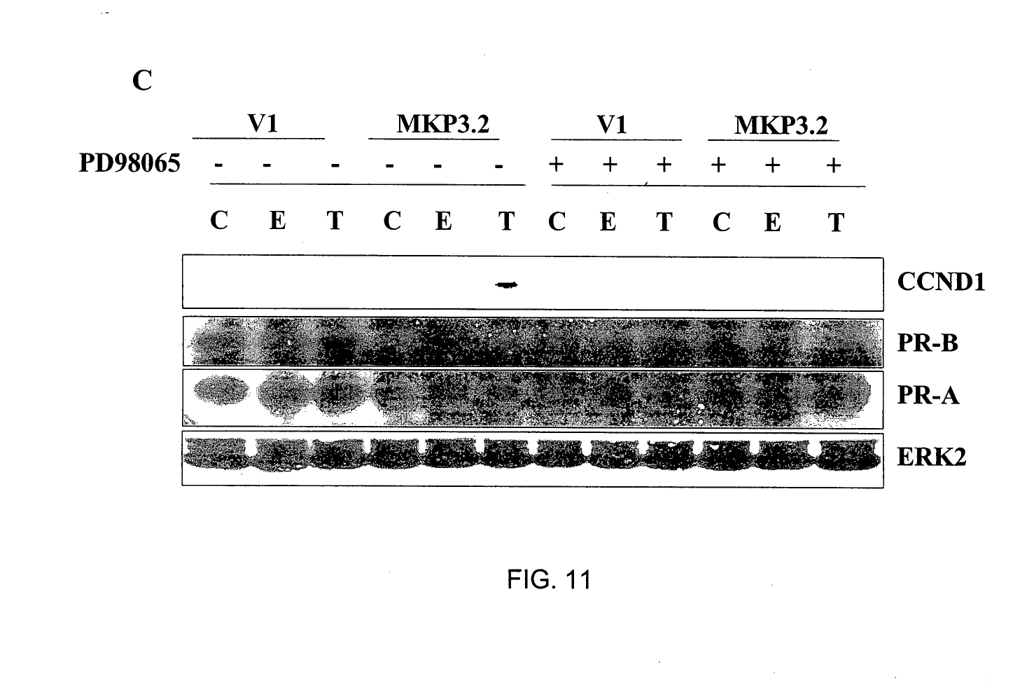

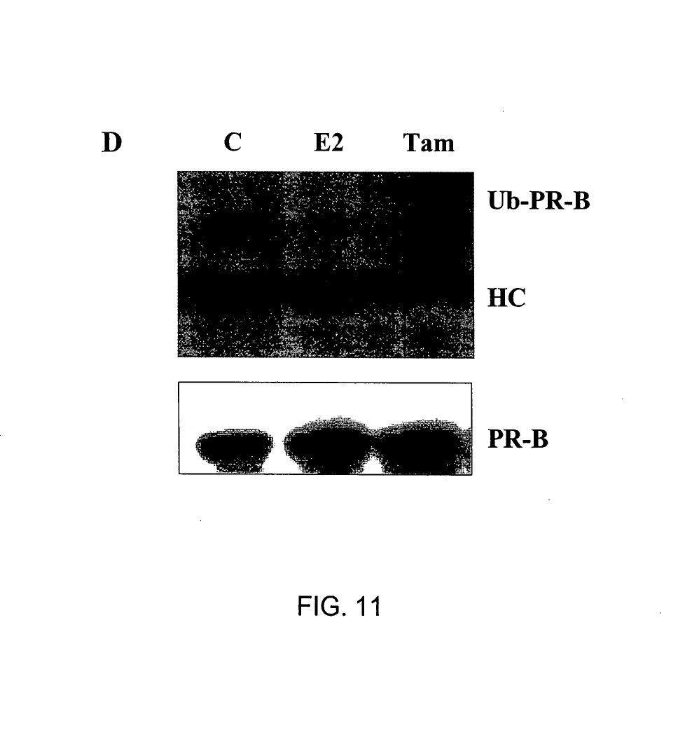

The cell cycle regulatory protein cyclin D1 (CCND1) is amplified and/or overexpressed in breast cancer, and higher levels have been associated with TR in the clinical setting (Stendahl et al., 2004). CCND1 is an estrogen-induced protein, and is also a downstream marker of activated MAPK signaling in breast cancer cells (Doisneau-Sixou et al., 2003). High levels of CCND1 were found in Tam-treated MKP3 overexpressing cells using immunoblot analysis (FIG. 1I A, top panel); densitometric scanning of the CCND1 immunoblot is shown in FIG. 11B. Thus, the Tam-stimulated soft agar growth and xenograft growth that was observed concomitant with MKP3 overexpression was coupled with the induction of the MAPK downstream marker of proliferation, CCND1.

Similarly, the progesterone receptor (PR) A and β forms are induced by estrogen, and recently reported that a change in the ratio of PR-A to PR-B in breast cancer predicts for a poor response to adjuvant Tam therapy (Hopp et al., 2004). As expected with two hours of estrogen treatment, no induction of the two PR isoforms were detected in either the control vector (Con 1 and 2), or in the MKP3-transfected cells (FIG. 11A). However, there was a striking diminution of PR-B levels in the two MKP3 transfectants treated with Tam, which was not observed in the control MCF-7 transfectants. In contrast, the levels of the estrogen-inducible amplified in breast (AIB) ER coactivator protein (Anzick et al., 1997) were unchanged with MKP3 overexpression; P190 was used as a loading contol in these experiments (lower panel). Furthermore, it was found that the MEK1,2 inhibitor PD98059 blocked tamoxifen induction of CCND1, and restored PR-B levels in MKP3 overexpressing cells (FIG. 11C).

How might MKP3 be influencing PR-B levels? It has been shown that progestin-mediated degradation of PR-B occurs by a mechanism involving pMAPK. Therefore, PR ubiquitination in MKP3 overexpressing cells was examined (FIG. 11D). To demonstrate ubiquitin-conjugated PR-B, HA-tagged ubiquitin and PR-B were transiently overexpressed in HeLa cells by cotransfection of the two plasmids. Cells were treated with E2 or Tam for 2 hrs in the presence of a proteasomal inhibitor to allow for the accumulation of PR-ubiquitin conjugates. PR-B was then immunoprecipitated and visualized by immunoblotting with HA-specific antibody. The level of polyubiquitin-conjugated PR-B was increased in Tam-treated cells which correlated with the lower steady state levels of PR-B in MKP3 overexpressing cells. These results suggest that the Tam-resistant phenotype of the MKP3 overexpressing cells is associated with two biomarkers of clinical resistance, elevated CCND1 and altered PR-A to PR-B ratios, downstream of MAPK activation.

### SUMMARY

The development of tamoxifen resistance (TR) is most frequently associated with the continued presence of ERα at the time of tumor progression (Encamacion et al., 1993). One current hypothesis is that ERα remains essential to the problem of resistance, due to its molecular crosstalk with growth factors, and/or downstream intracellular signaling molecules. Support for this hypothesis is garnered by data showing that overexpression of specific genes into ERα-positive cells, such as the growth factor receptors EGFR (Knowlden et al., 2003) and HER-2/neu (Benz et al., 1993), the receptor tyrosine kinase EphA2 (Lu et al., 2003), the tyrosine kinase Akt-3 (Faridi et al., 2003), and the ERα coactivator AIB (Loui et al., 2004), all promote tamoxifen-resistant growth. Recently, the growth factor receptor tyrosine kinase inhibitor gelfitinib, and HER-2/neu receptor blocking antibodies have been used to restore tamoxifen's growth-inhibitory effects in Tam-resistant breast cancer cells (Moulders et al., 2001; Shou et al., 2004) There is also a growing body of evidence implicating the mitogen-activated protein kinase extracellular signal-regulated-kinases ERK 1,2 MAPKs in the growth factor phosphorylation cascade, and its interaction with ERα signaling in TR (Kurokawa et al., 2000). Indeed, phosphorylation of ERK 1,2 MAPK has been associated with a poorer quality of response to tamoxifen in breast cancer patients (Gee et al., 2001).

It is known that ERα can be phosphorylated by activated MAPK, resulting in ligand independent ER activity (Kato et al., 1995). An emerging area of research in MAPK signaling is the role of specific protein phosphatases in the control of MAPK activation, and their role in specific biological responses. MAPK phosphatase 3 (MKP3, also called dual specificity phosphastase 6 DUSP6 and Pyst1) is a member of a phosphatase family that inactivates MAPK function by dephosphorylating both phosphoserine/threonine and phosphotyrosine residues [reviewed in (Camps et al., 2000)]. MKP3 is in a regulatory feedback loop with ERK 1,2 MAPK because it is both activated by binding to ERK2, and reciprocally inactivates this MAPK (Zhou et al., 2001).

### Example 5

### Significance of MKP3 Embodiments

Tam has been the most frequently prescribed antiestrogen for the treatment of women with early-stage and metastatic ERα-positive breast cancer. Although many patients will initially benefit from Tam treatment, the emergence of resistance is a major clinical problem. ERα and PR status have been used for over 30 years to predict response to Tam in the clinical setting (Early Breast Cancer Trialists' Collaborative Group, 1998; Bardou et al., 2003). Recent evidence demonstrates the activation of HER-2 and/or ERα coregulatory proteins, such as AIB1, in a subset of patients with Tam resistance (Osborne et al., 2003; Gutierrez et al., 2005). There is also preclinical evidence that a number of diverse receptor tyrosine kinases, such as the EphA2 receptor (Lu et al., 2003) and the epidermal growth factor receptor (Knowlden et al., 2003), or intracellular signaling molecules AND-34/BCAR3 (Felekkis et al., 2005), AKT3 (Faridi et al., 2003) and pMAPK (Gee et al., 2001) could be significant in the development of resistance to hormone therapy. It has been hypothesized that these pathways might impact on ERα activity, and hence Tam effectiveness. It has also been shown that PKA activation, via down-regulation of a negative regulator of PKA (PKA-R1α), is associated with resistance through signaling to ER α (Michalides et al., 2004). Thus, there are multiple feedback systems between ER α and other intracellular signaling effectors which can contribute to resistance, and have examined herein a MAPK signaling network which contributes to the therapeutic response of breast cancer cells. Increased dependence on MAPK signaling has been previously demonstrated to be important for both TR and adaptive resistance to estrogen deprivation in MCF-7 cells (Larsen et al., 1999; Song et al., 2002). The inventors identified MKP3, a negative regulator of MAPK, as being expressed at higher levels in Tam-resistant metastatic lesions, and demonstrated in preclinical studies that its overexpression can confer Tam-resistant growth of MCF-7 cells in vitro or as xenografts in athymic nude mice.

The inventors employed microarray expression profiling to identify genes associated with Tam resistance in breast cancer patients as a means of exploring new regulatory mechanisms operative during the selective pressure of Tam treatment. Microarray studies are increasingly being employed to develop prognostic and predictive models of patient outcome in breast cancer (Chang et al., 2005). Recently a two-gene expression Tam prediction model was developed using microarray analysis comparing primary breast tumors from patients treated with adjuvant Tam who remained disease-free, to those patients who developed distant recurrence (Ma et al., 2004). Although these results have been recently challenged (Reid et al., 2005), microarray technologies have shown great promise in identifying molecular features of hormone responsiveness (Jansen et al., 2005). However, the development of reliable predictive models will undoubtedly require large sample sizes due to the heterogeneous nature of breast cancer, and the multifactoral problem of treatment resistance. The present invention differed in both experimental design and goal compared to the microarray study of Ma et al. (2004). Although the inventors used a similarly-defined Tam sensitive group of primary tumors, they chose to compare these to metastatic lesions from patients who recurred while on Tam, with the goal to identify gene candidates that could then examine further for a mechanistic role in resistance. They did not seek to identify a predictive Tam response expression profile due to the small number of metastatic lesions available for analysis. It is unfortunate that metastatic lesions are infrequently biopsied for diagnostic purposes in recurrent breast cancer, which will ultimately limit the development of reliable predictive studies with these lesions.

Neither Ma et al. (2004), or a similarly designed microarray study reported by Jansen et al. (2005) found MKP3 RNA levels to be differentially associated with the outcome of Tam-treated primary tumors. This difference can be attributed to either experimental design, or in the diverse array platforms utilized between the studies. Interestingly, it was found that the levels of pMAPK protein were highest in Tam-treated and lowest in the presence of estradiol when MCF-7 cells were engineered to overexpress MKP3. Furthermore, it was found that MEK inhibition reversed Tam-induced soft agar growth of MKP3 overexpressing cells, further implicating MAPK signaling in resistance in the model. This result indicates that activated MAPK remains a common component of Tam-resistant growth in the preclinical model.

The inventors demonstrated that MKP3 enzymatic activity was particularly sensitive to regulation by hormones, and propose that its hormonal modulation may be an alternative and novel mechanism by which ERK1,2 can be activated and regulated in breast cancer cells. Several of the MKPs are known to be induced following exposure to stress and/or growth factor stimulation (Camps et al., 2000), however MKP3 has not been previously demonstrated to be regulated by these stimuli. MKP3 has been shown to be transcriptionally up-regulated after activation of the ERK2 pathway (Camps et al., 1998). The control of MKP3 activity at the post-transcriptional level is not well understood, however a direct physical interaction with ERK2 is known to increase its activity several fold (Zhou et al., 2001). In specific embodiments, MKP3 is a novel target of Tam action in breast cancer cells in patients following prolonged MAPK activation during adjuvant Tam treatment. It is possible that tumors may compensate for chronic activation of MAPK by up-regulation of phosphatases, such as MKP3, that control these pathways. The emergence of Tam resistance may therefore involve the disruption of this regulatory compensatory loop by inactivation of MKP3 phosphatase activity explaining the seemingly paradoxical up-regulation of MKP3 levels, but down-regulation of its activity in Tam-treated MKP3 overexpressing cells. Since MKP3 has been shown to be a relatively unstable protein (Warmka et al., 2004), it may be a particularly susceptible target during breast tumorigenesis.

Sustained activation of MAPK has been observed in a number of systems, and does not always correlate with upstream Ras-Raf-MEK1,2 activities. For instance, carcinogenic activation of ERK1,2 in human lung cancer cells triggers MKP1 degradation via the ubiquitin-proteasome pathway (Lin et al., 2003). Constitutive induction of pERK1,2 in the senescence of human diploid fibroblasts has been shown to be associated with reduced MKP3 and protein phosphatase 1/2 activities (Kim et al., 2003). Similar to this above report, found that there were no differences in the levels of MAPK bound to MKP3 in the MKP3 overexpressing cells, irrespective of hormonal stimulation, suggesting that pMAPK levels could be explained by a modulation of MKP3 activity, and perhaps influences on other as yet unidentified phosphatases.

There is also evidence for multiple, temporally discrete pathways which differentially regulate MAPK depending on the external stimulus (Grammer and Blenis, 1997). For instance, phosphatidylinositol-3 kinase (PI3K) and protein kinase C (PKC) isoforms have been shown to be important for MEK-independent, sustained MAPK activation in Swiss 3T3 fibroblasts (Grammer and Blenis, 1997). Fibroblast growth factor (FGF) 1 and heregulin 1-induced TR in MCF-7 cells is also associated with prolonged MAPK activation that is incompletely susceptible to MEK inhibitors (Thottassery et al., 2004). The results indicate that Tam increases ERK1,2 activity via the loss of MKP3 phosphatase activity, an alternative “off-off” mechanism of resistance which remains sensitive to MEK inhibition. Therefore, patients with Tam-resistant disease and elevated MKP3 may be markedly sensitive to MEK inhibitors.

ERα expression is lost in a minority of recurrent metastatic lesions after Tam treatment (Gutierrez et al., 2005). The retention of ERα suggests that it may continue to play a role during the development of resistance. ERα levels were unchanged when overexpressed MKP3 in MCF-7 cells, and displayed the expected estrogen regulation. This is in contrast to that seen for ERα levels when various growth factor signaling components are overexpressed in MCF-7 cells. For instance, overexpression of constitutively active forms of Raf-1 and MEK1, which activate pMAPK, leads to a down-regulation of ERα that can be reversed with MEK inhibitors (Oh et al., 2001). Recently, it has been shown that nuclear factor-κB may be partially involved in the down-regulation of ERα with activated Raf-1 and MEK1 (Holloway et al., 2004). Thus, the model is different from hyperactivation of the Raf1/MEK1 signaling pathway on ERα, and potentially reflects the more common resistance mechanisms associated with continued ERα expression in patients.

There are a variety of phosphorylation sites within ERα which modulate a number of different functions, such as transcriptional activity and hormonal sensitivity, which are sites for cross-talk with signal transduction pathways. The inventors have shown that PKA signaling induces ERα S305 phosphorylation, which is coupled to acetylation at K303 within the hinge domain and estrogen sensitivity (Cui et al., 2004). This site has been demonstrated to be important for Tam resistance (Michalides et al., 2004) and expression of CCND1 (Balasenthil et al., 2004). A major site of phosphorylation in response to estrogen is ERα S118, which is located in the hormone-independent, activation function (AF)-1 region of the receptor (Le Goff et al., 1994), although this finding has been disputed by the inability of some investigators to induce pMAPK with estrogen in different MCF-7 sublines (Lobenhofer and Marks, 2000). The ERα S118 site is also phosphorylated in response to epidermal growth factor signaling, possibly via pMAPK (Bunone et al., 1996). There is in vitro evidence that S118 is phosphorylated by activated ERK1,2 in breast cancer cells (Kato et al., 1995). ERα S118 was phosphorylated in response to Tam in MKP3 overexpressing cells, which was associated with higher pMAPK levels. This indicates that ERα pS118 may be a marker of resistance in the model. However, high levels ERα pS118 have been shown to be associated with a better disease outcome in breast cancer patients treated with Tam in one clinical study (Murphy et al., 2004). Therefore the usefulness of pS118 as a clinical marker of resistance requires further study. There is some evidence that estrogen-induced phosphorylation of ERα S118 may be independent of ERK1,2 activation, increasing the complexity in dissecting the role of S118 in ERα function (Joel et al., 1998).

Up-regulation of CCND1 via ERα signaling is associated with an increased proliferation response in breast cancer cells (Prall et al., 1998), and CCND1 overexpression is predictive of TR in patients (Stendahl et al., 2004). It was found that CCND1 was elevated in Tam-treated MKP3 overexpressing cells, which is consistent with the above experimental and clinical data. PR status is also a useful predictive factor for Tam response (Bardou et al., 2003). Approximately 30% of patients are ERα-positive, but PR-negative, and respond poorly to Tam (Bardou et al., 2003). The molecular mechanisms associated with the resistant phenotype of PR-negative patients is not understood, but there is some evidence to indicate that low PR levels may be the result of elevated growth factor signaling (Cui et al., 2003). It was recently reported that patients with low PR-β-form expression were significantly more likely to relapse with Tam therapy (Hopp et al., 2004). The previous clinical study is consistent with the finding herein that levels of PR-B were lower in Tam-treated MKP3 overexpressing cells. Hyperactivation of MAPK in Tam-treated MKP3 transfectants resulted in an increase in ubiquitination of PR-B, a result confirming data showing that activated MAPK signals the degradation of PR by the 26S proteasome (Lange et al., 2000).

Activation of JNK with MEK1 inhibitor treatment of MCF-7 cells was detected, with further enhanced JNK phosphorylation noted in the MKP3 overexpressing cells. There was no observation of activation of p38 MAPK. In specific aspects of the invention, the molecular mechanism associated with these enhanced pJNK levels is further characterized, but it does not appear to be associated with decreased levels of MKP1, since MKP1 levels were unchanged either with MEK inhibitor treatment, or MKP3 overexpression. Whether down-regulation of other MKPs may be associated with this effect, and whether combined MAPK inhibitors with Tam treatment are efficacious in the model is investigated.

Many patients with ERα-positive tumors unfortunately fail Tam therapy. There is a critical need to find biomarkers which accurately identify those patients who will not benefit from Tam treatment. The results of the Arimidex or Tamoxifen Alone or in combination (ATAC) study demonstrated a major benefit for Arimidex in the ERα-positive, PR-negative subgroup of patients compared to Tam treatment alone (Baum et al., 2002; Dowseft, 2003). In specific aspects of the invention, MKP3 expressing tumors might similarly be sensitive to estrogen deprivation (Arimidex treatment), given the finding that there was limited growth of MKP3 overexpressing cells in the absence of estrogen. In summary, this invention indicates that at least MKP3 is an attractive new diagnostic and therapeutic target in breast cancer.

### Example 6

### Expression of EBP50 is Associated with Resistance to Tamoxifen

Estrogen is critical for mammary gland development and implicated in breast cancer tumorigenesis and progression, thus the disruption of estrogen signaling via targeting its receptor (ER) is a promising treatment strategy for breast cancers. Tamoxifen (Tam) is the most frequently prescribed antiestrogen for the prevention and treatment of ER positive breast cancers, but while Tam is initially useful in many breast cancer patients, metastatic lesions would recur during TAM treatment, defined as acquired tamoxifen resistance (TR). Current studies suggested that acquired TR is associated with downregulation of estrogen-induced genes, such as progesterone receptors, and/or overexpression of estrogen-repressed genes, such as AIB1 and HER-2/neu. The inventors and others have found that estrogen also regulates the expression of a number of cytoskeleton organizers, such as EBP50, Ezrin, and moesin in breast cancer cells, but their involvement in TR has not been studied. Estrogens and antiestrogens are known to impact on cell cytoarchitecture by affecting adhesion structures and the rearrangement of intermediate and actin filaments (Sapino A., et al. 1986.) Estrogens are known to increase, and antiestrogens like tamoxifen, decrease the expression of the sodium hydrogen exchanger regulatory factor NHE-RF, also known as ezrin binding protein 50 (EBP50) (Ediger T R, et al. 1999. EBP50 acts as a multifunctional adaptor/scaffolding protein and it may play a role in signal transduction pathways and the cytoarchitecture in breast cancer (Stemmer-Rachamimov, A O, et al. 2001.

To explore whether expression of these genes are associated with resistance, expression profiled study two groups of tumors: Tam Sensitive (TS, n=5) were primary tumors from patients who were treated with Tam and had not experienced a recurrence within 7-10 years of follow up, and TR tumors (n=5) were metastatic breast tumors from patients who were treated with Tam and whose metastatic lesions recurred while on treatment. The inventors found that EBP50 expression was significantly downregulated in the TR group of tumors. Furthermore, EBP50 expression was downregulated in a TR cell line model which was generated from ER-positive T47D breast cancer cell line genetically engineered to overexpress metastasis-associated protein 2 MTA2. These data suggest that EBP50 expression is inversely related to resistance, and suggest that cytoskeleton reorganization and/or signaling may be important for the development of TR.

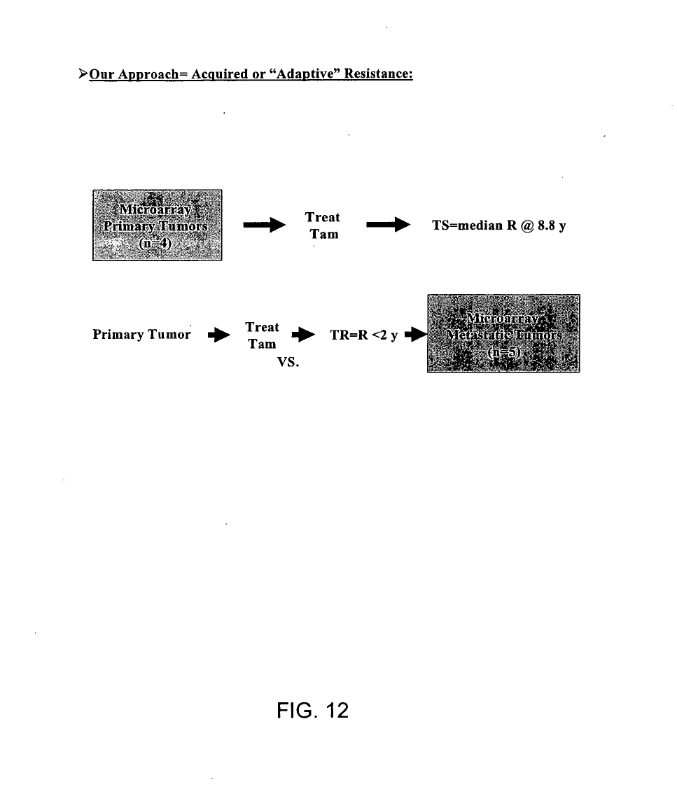

FIG. 12 shows identification of exemplary altered gene expression associated with tamoxifen resistance in breast tumors. Recently investigators have used microarray profiling of primary tumors to identify genes whose expression is associated with response to tamoxifen. The inventors have taken a different approach and have compared the gene expression profiles of primary tumors compared to metastatic tumors which have arisen in spite of tamoxifen treatment. The approach was aimed at identifying altered genes expression associated with acquired or adaptive resistance.

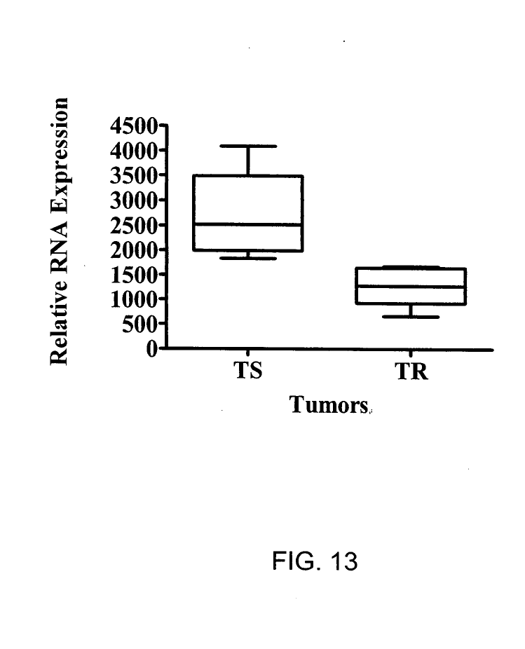

FIG. 13 shows comparison of EBP50 RNA levels in tamoxifen-sensitive and tamoxifen-resistant breast tumors. EBP50 levels were determined using Affymetrix U95A human GeneChip arrays employing dChip for normalization and estimation of expression values. The mean levels of EBP50 were reduced in the tamoxifen-resistant (TR) tumors compared to tamoxifen-sensitive tumors (TS). This difference was statistically significant, with p=0.02 using a t-test.

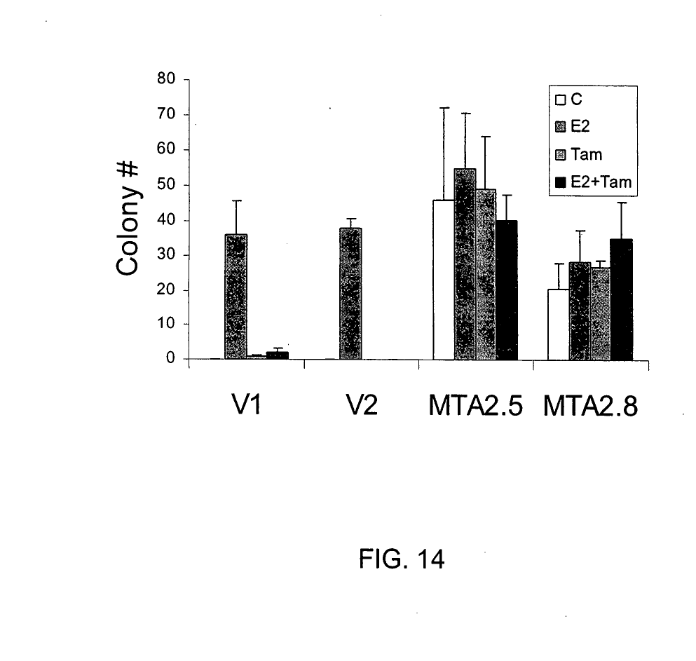

FIG. 14 shows overexpression of MTA2 in T47D cells is associated with hormone-independent and tamoxifen-resistant growth in soft agar. Metastasis associated protein 2 (MTA2), also known as PID, is contained in nucleosome remodeling and histone deacetylation (NuRD) complexes. Overexpression of MTA2 into T47D human breast cancer cells (MTA2.5 and 2.8 clones) increases the growth in soft agar in the absence of estrogen (C). These cells were unresponsive to either estrogen-stimulation (E2), or to the growth inhibitory effects of tamoxifen (Tam), compared to vector alone transfected cells (V1 and V2).

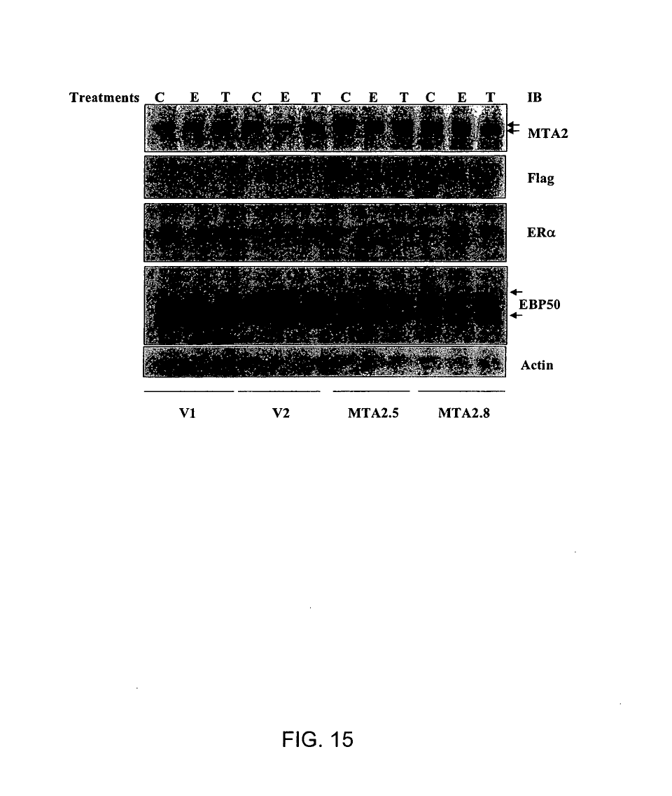

FIG. 15 demonstrates decreased expression of EBP50 in MTA2 overexpressing T47D cells. The MTA2 expression vector was tagged with a Flag epitope. Immunoblot analysis of two vector controls (V1 and V2) and two MTA2 transfected clones demonstrated that MTA2 was indeed overexpressed and that estrogen (E) and tamoxifen (T) treatments did not affect its levels. As expected, levels of ERα protein were decreased by estrogen. EBP50 levels were increased by estrogen. Concomitant with MTA2 overexpression, EBP50 levels were decreased. Thus, EBP50 levels were reduced in TR cells. However these results are only correlative, and one can examine whether EBP50 is directly involved in resistance using siRNA technologies, for example.

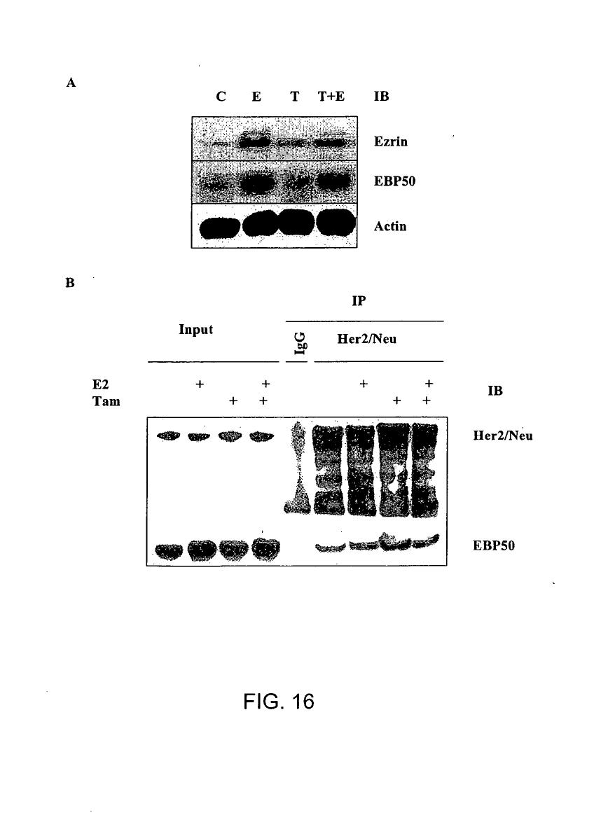

FIGS. 16A and 16B demonstrate that EBP50 binds to HER2. MCF-7 breast cancer cells were treated with estrogen (E), tamoxifen (T), or E+T for 24 hours, and levels of ezrin and EBP50 were examined using immunoblot analysis (Panel A). Both ezrin and EBP50 levels were similarly induced by estrogen. In Panel B, immunoprecipitation of E or T treated MCF-7 cells was performed followed by immunoblot analysis. These results demonstrate that EBP50 and HER-2 interact in the absence and presence of E or T.

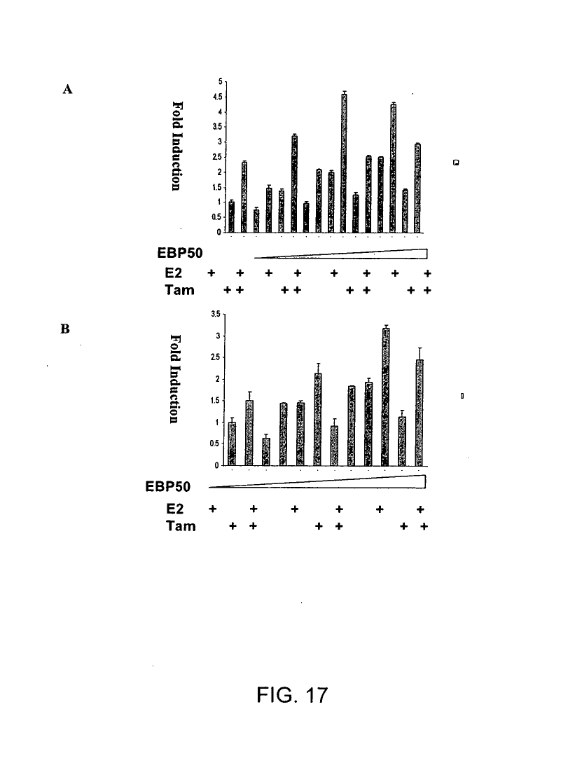

FIGS. 17A and 17B show that EBP50 overexpression enhances ERα activity. Transient transactivation assays with an ERE-luciferase reporter were utilized in U20S cells expressing exogenous ER (Panel A), or MCF-7 cells expressing endogenous ER (Panel B). ER activity was enhanced with EBP50 expression in the presence of estrogen. Tamoxifen was an antagonist and reversed estrogen's effects.

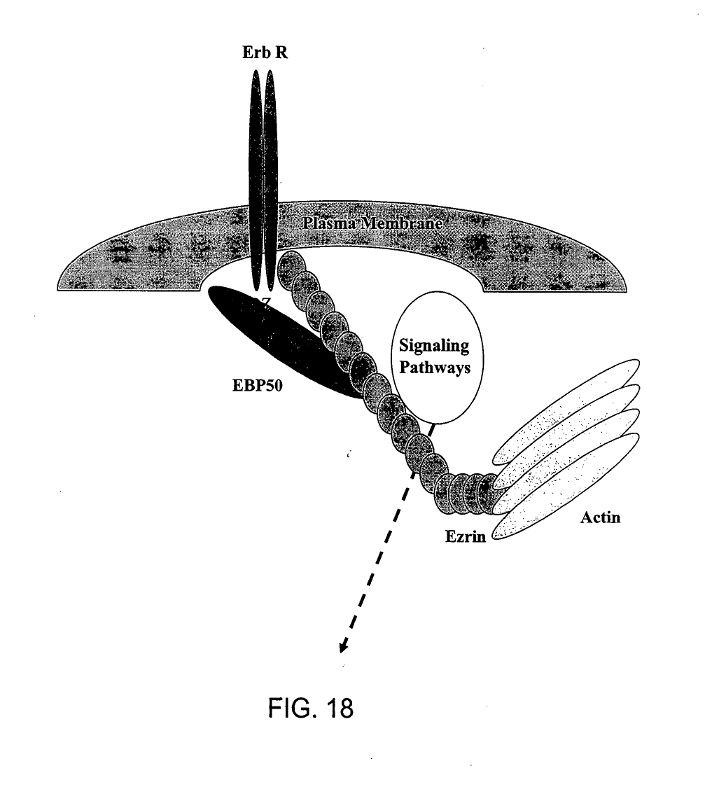

FIG. 18 shows an exemplary model for the role of EBP50. In exemplary embodiments of the invention, a reduction in EBP50 levels leads to an increase in HER-2 receptor levels, influences downstream signaling pathways, and/or affects cytoskeletal architecture. In the exemplary model, EBP50 is a negative regulator that is lost during acquired tamoxifen resistance.

Thus, lower levels of EBP50 were associated with resistance to the antiestrogen tamoxifen in tamoxifen-resistant breast tumor recurrences, and plays a role in a novel model of resistance generated by overexpression of MTA2 in T47D cells, in specific embodiments of the invention. This indicates that cytoskeleton reorganization, such as through EBP50 modulation, for example, may be involved in resistance. The inventors also found that EBP50 and HER-2 interact in MCF-7 breast cancer cells, and in specific embodiments of the invention EBP50 levels regulate HER2 receptor signaling, such as due to alterations in receptor levels, or receptor internalization and recycling, similar to that demonstrated for EBP50 regulation of G protein-coupled receptors.

### Example 7

### RHOA Inhibitor, RHOGDIA, is a Substrate for CBP/P300 Histone Acetyl Transferase Activity

In response to both cellular and extracellular signals, estrogen receptor (ER) regulates gene expression with cellular functions through its crosstalking with coregulators. It has been shown that an inhibitor of guanine nucleotide exchange and activativation of RhoGTPases, RhoGDI, is a modulator of ER function with its mechanism remaining unclear. In particular, RhoGDI is an inhibitor of the Rho A family of GTPases, and the expression and activation of RhoA is associated with breast cancer progression (Fritz et al., 1999; Fritz et al., 2002). RhoGDI's effect on ER transcriptional activity is dependent on ER-regulatory proteins (co-activators).

The inventors demonstrate that RhoGDI is associated with ERα in breast cancer cells through indirect interaction(s), because in vitro binding was not observed between ERα and RhoGDI; overexpression of RhoGDI inhibits wild-type ERα ligand sensitivity in both Hela and MCF-7 cells. Thus, in specific embodiments RhoGDI modulates ERα activity through interaction with other ERα cofactors. To further characterize this, the inventors demonstrated that RhoGDI is a substrate for p300 histone acetyl transferase (HAT) activity. They also demonstrated that their in vivo association can be blocked by Tamoxifen, which modulates the association of ERα with cofactors. Finally, overexpression of RhoGDI dramatically inhibited the in vivo acetylation of ERα. Taken together, these findings indicate that RhoGDI is a substrate of p300/CBP family members and represses ERα ligand sensitivity through competing p300 HAT activity.

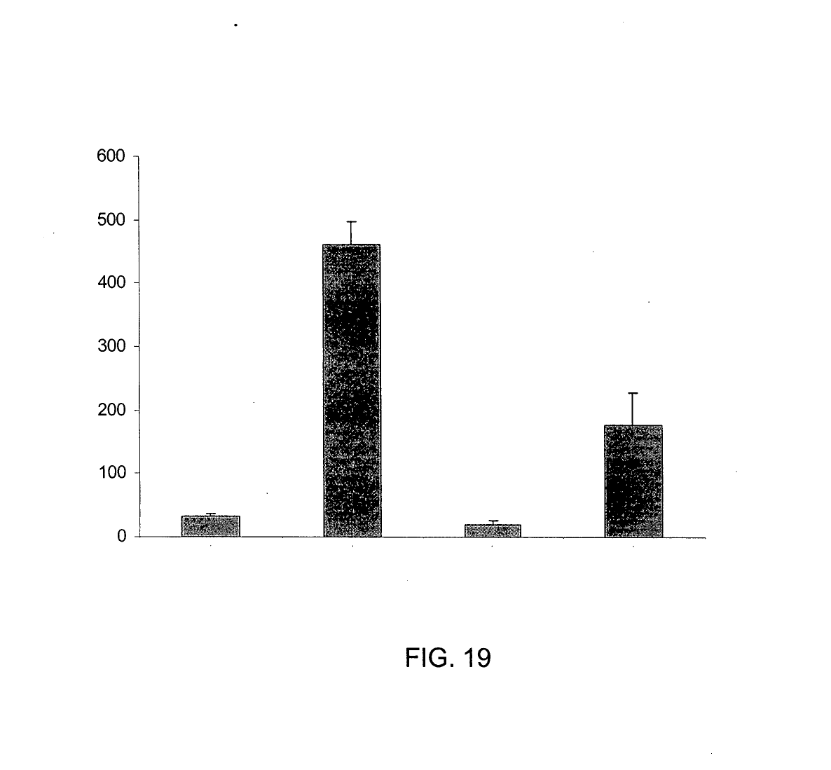

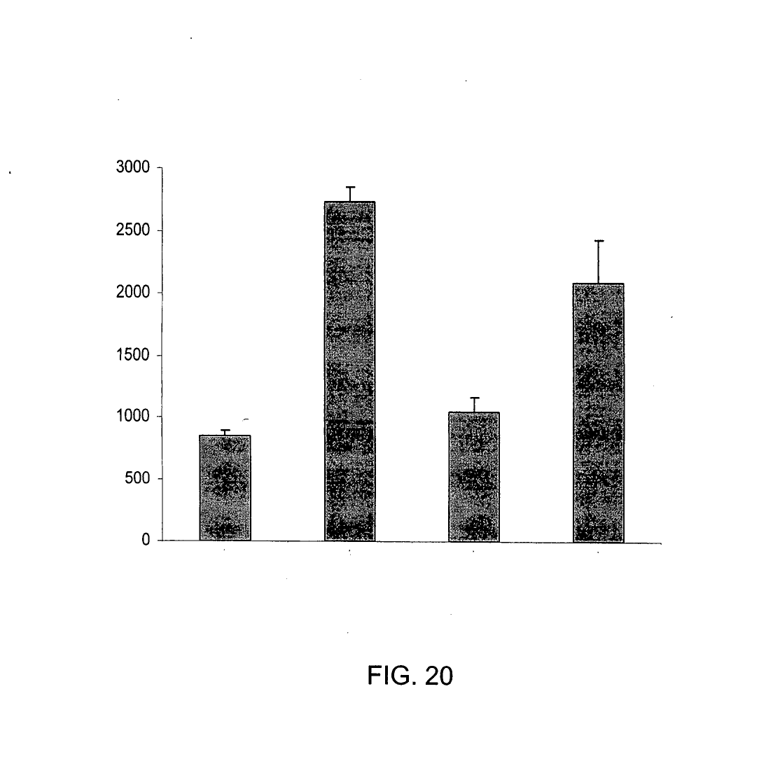

Thus, in one embodiment of the present invention, the inventors investigated whether RhoGDI affects ERα activity. FIG. 19 shows that RhoGDI represses exogenous ERα activity in Hela cells. HeLa cells were transiently transfected with an ERα expression vector only, or in combination with a RhoGDI expression vector. ERα activity was measured with the cotransfected ERE-tk-Luc reporter, and transfection efficiency were normalized to co-transfected β-Gal activity. Expression of RhoGDI reduced ER activity. FIG. 20 shows that RhoGDI represses endogenous ERα activity in exemplary MCF-7 breast cancer cells.

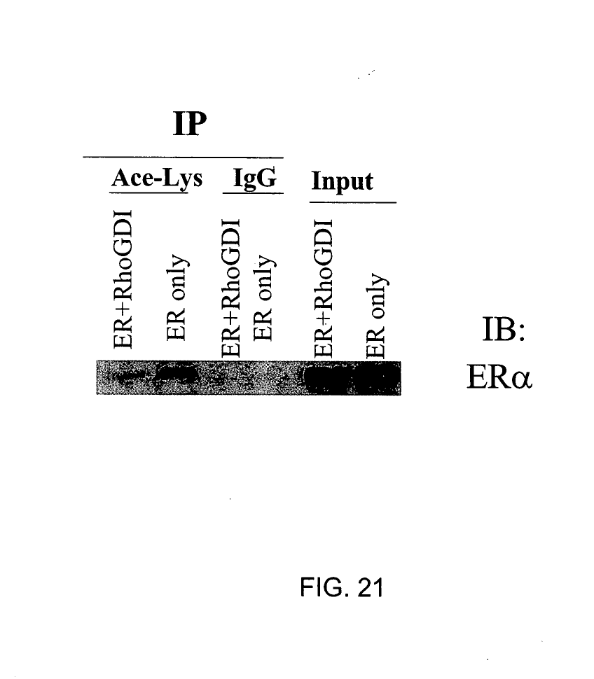

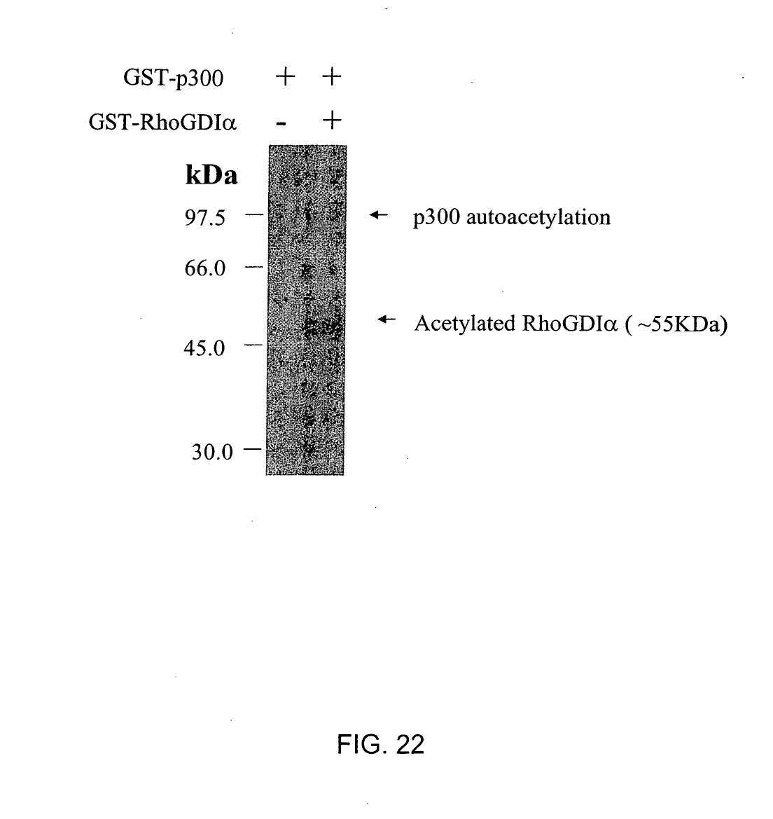

The inventors also characterized the mechanism underlying RhoGDI's repression of ERα activity. FIG. 21 shows that RhoGDI decreases the acetylation of ERα level in vivo. HeLa cells were transiently transfected with either ERα alone, or in combination with RhoGDI. The cell lysates were immunoprecipitated (IP) with anti-acetylated lysine antibody, and the precipitates were then immunoblotted (IB) with an ERα antibody. Levels of acetylated ERα were reduced with RhoGDI expression. FIG. 22 shows that RhoGDIα is an in vitro substrate of p300 HAT activity. An in vitro acetylation assay was performed using purified GST-p300 HAT with either purified GST (negative control), or purified GST-RhoGDI in the presence of 14C-Actyl-CoA. The reactions were separated by SDS-PAGE, and visualized with autoradiography of the transferred ¹⁴C-Actyl. RhoGDI, like ERα, is acetylated by p300.

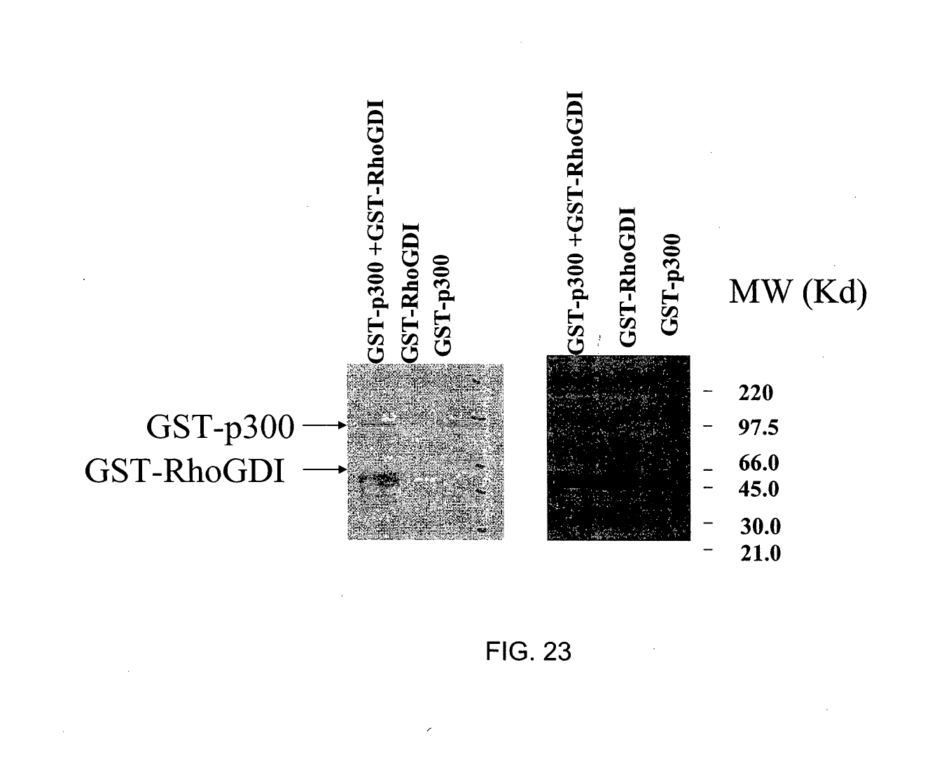

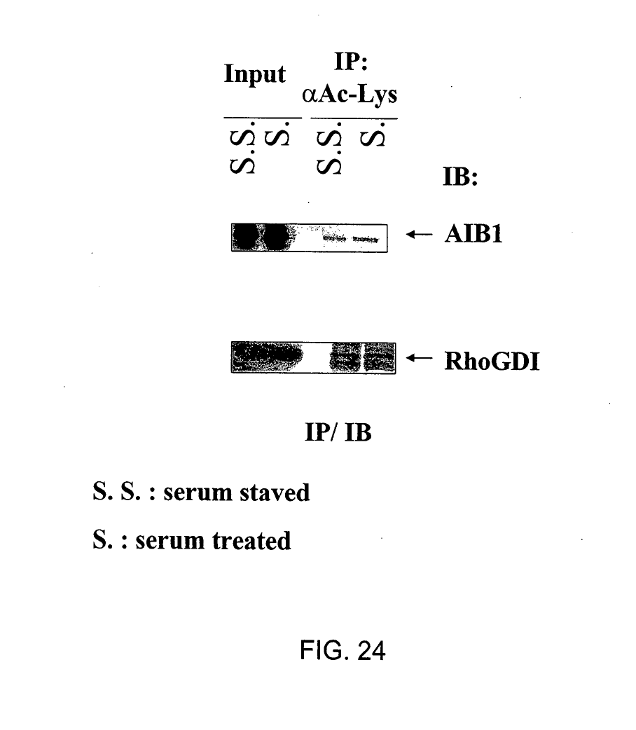

FIG. 23 shows that RhoGDI exhibits no intrinsic HAT activity. In vitro acetylation experiment was performed using GST-RhoGDI in combination with purified GST-p300HAT in the presence of ¹⁴C-Acetyl-CoA. The reactions were resolved onto SDS-PAGE and visualized with autoradiography of the transferred ¹⁴C-Actyl. The result indicates that RhoGDI itself has no intrinsic HAT activity. FIG. 24 shows that RhoGDI is acetylated in vivo. Cell lysates of MCF-7 cells grown in the presence or absence of serum were immunoprecipitated (IP) with anti-acetylated lysine antibody, and the precipitates were then immunoblotted (IB) with antibodies against either AIB1 or RhoGDI. The result indicates that RhoGDI is an acetylated protein in vivo.

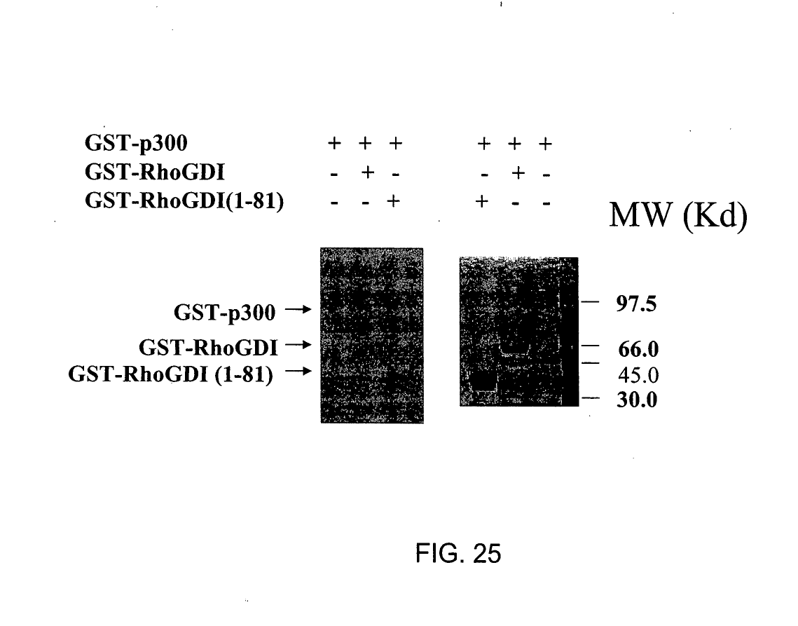

FIG. 25 demonstrates that the N-terminal region of RhoGDI comprises acetylation site(s). In vitro acetylation experiment was performed using GST-RhoGDI or GST-RhoGDI (aa:1-81) in combination with purified GST-p300HAT in the presence of ¹⁴C-Acetyl-CoA. The reactions were resolved onto SDS-PAGE and visualized with autoradiography of the transferred ¹⁴C-Actyl. The result indicates that N-terminal of RhoGDI contains acetylation site(s).

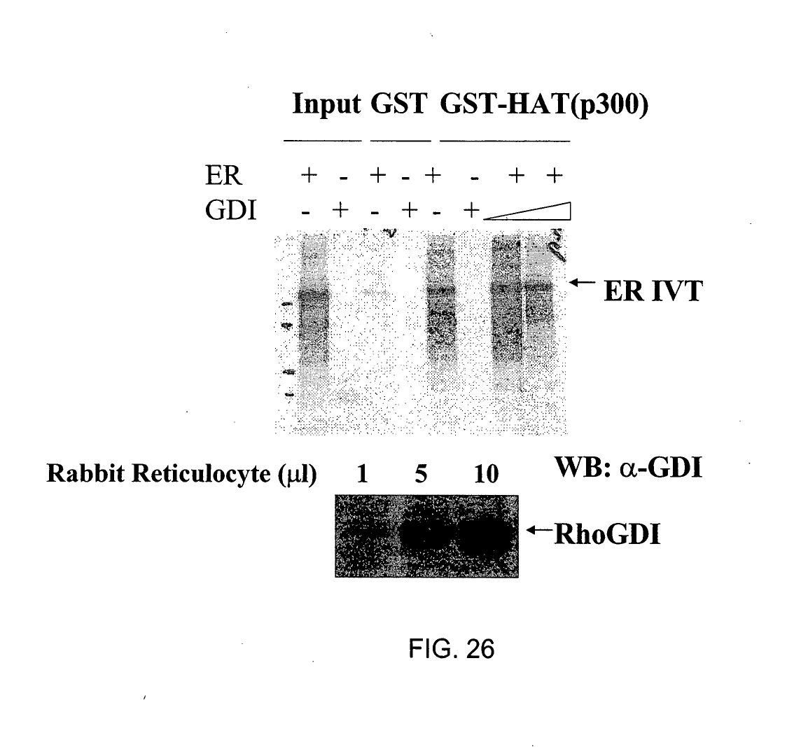

FIG. 26 shows that RhoGDI decreases ERα access to p300 HAT. GST-pull down experiment was performed to examine the effect of RhoGDI on the access of ER to p300 HAT. GSH-agarose immobilized p300HAT were incubated with in vitro translated, ³⁵S-incorporated ER in the presence of increasing amount of in vitro translated RhoGDI. The result indicates that RhoGDI decreases ER access to p300HAT.

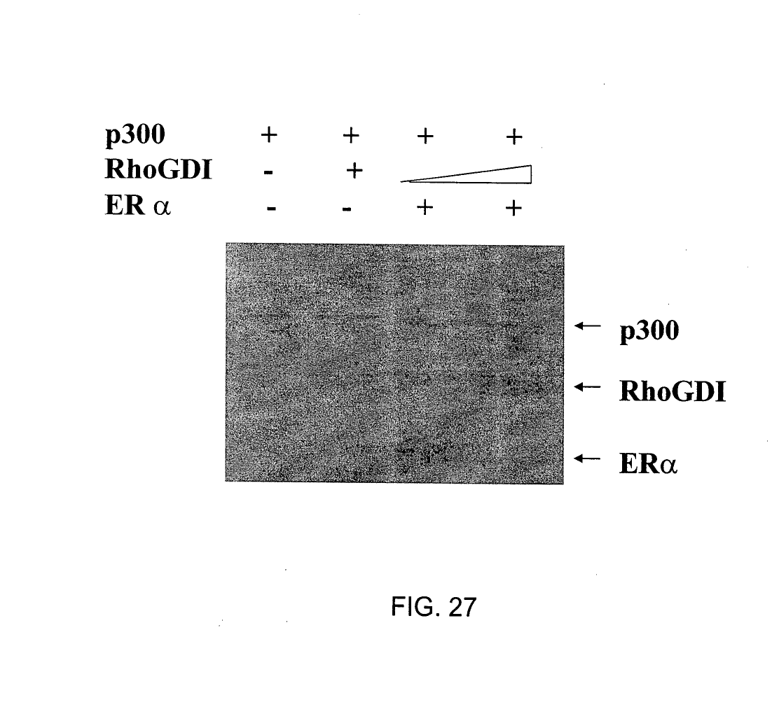

FIG. 27 demonstrates that RhoGDI inhibits p300 acetylation of ERα in vitro. Competitive in vitro acetylation assay was performed to examine the effect of RhoGDI on p300 acetylation of ER. Purified GST-ER was acetylated by GST-p300HAT in the presence of increasing amount of GST-RhoGDI. The result indicates that RhoGDI will compete p300HAT activity with ER.

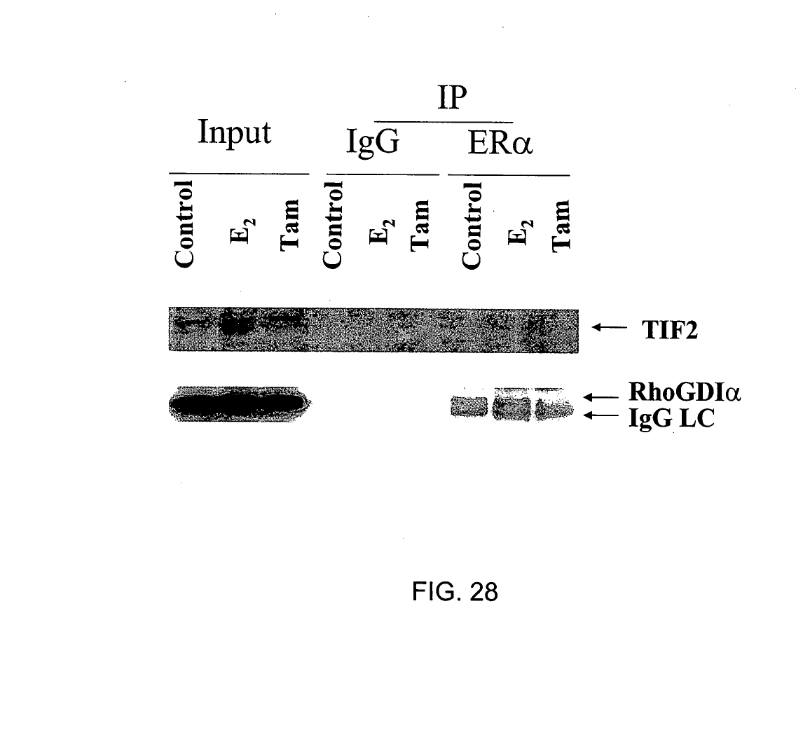

FIG. 28 illustrates that RhoGDI is associated with ER in vivo. Cell lysates of MCF-7 cells treated with or without E2 or tamoxifen serum were immunoprecipitated (IP) with anti-ER antibody, and the precipitates were then immunoblotted (IB) with antibodies against TIF2 or RhoGDI. The result indicates that association of RhoGDI with ER is disrupted in the presence of Tamoxifen.

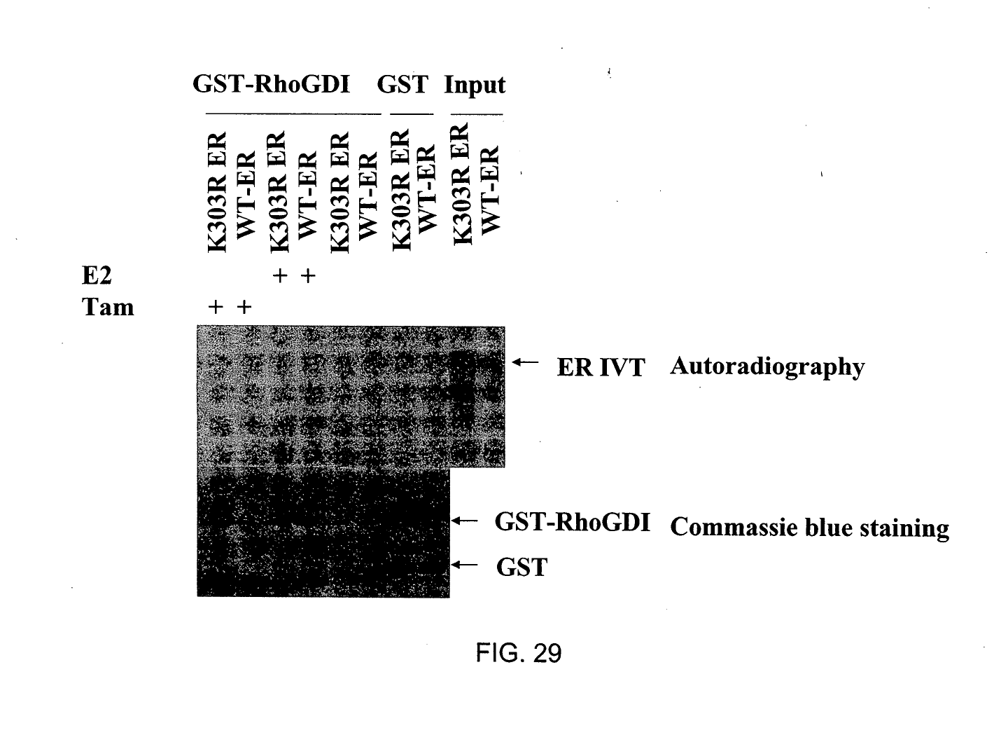

FIG. 29 shows that RhoGDI does not bind to ER directly. GST-pull down experiment was performed to examine the direct interaction between ER and RhoGDI with or without treatment of E2 or Tamoxifen. GSH-agarose immobilized p300HAT were incubated with in vitro translated, ³⁵S-incorporated ER in the presence of increasing amount of in vitro translated ER in the presence of E2 or Tam. The result indicates that RhoGDI does not interact with ER directly.

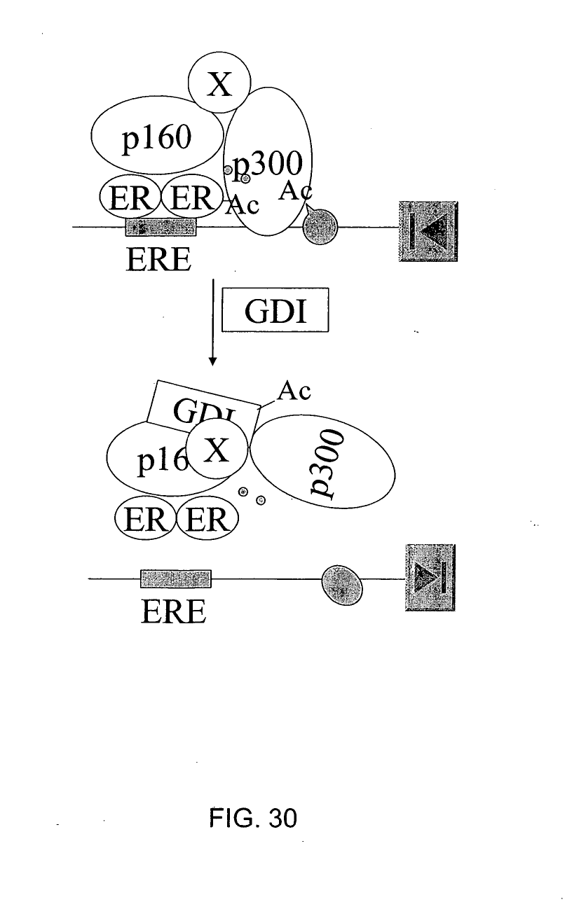

FIG. 30 illustrates an exemplary model for a role of RhoGDI associated with ER. In conclusion, RhoGDI is a substrate of p300 in vitro, and acetylated in vivo RhoGDI represses ER activity through competition of p300 HAT activity; this mechanism affords resistance to tamoxifen, in specific embodiments of the invention.

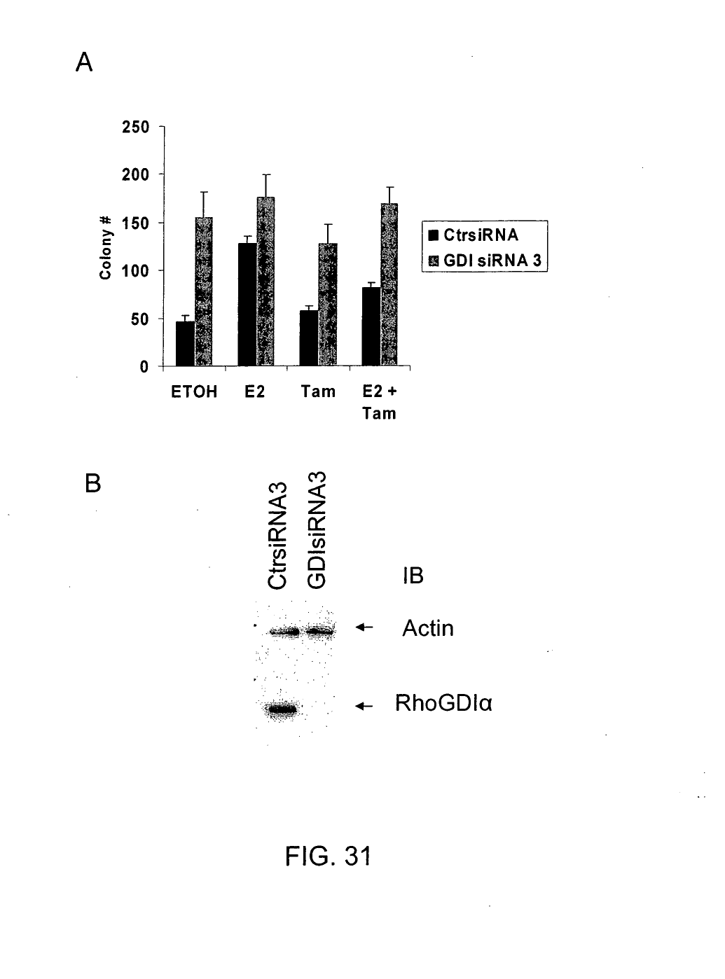

FIGS. 31A and 31B shows that RhoGDI confers resistance to tamoxifen. In the patients, Rho GDI was lower in TR, so the inventors used an siRNA directed to RhoGDI to demonstrate enhanced growth in soft agar in the presence of tamoxifen. This is analogous to the study utilizing overexpression of mkp3 in Example 2.

### Example 8

### Targeting Polynucleotides of the Present Invention

In particular aspects of the invention, one or more of the polynucleotides of the present invention are targeted, such as for cancer therapy. In specific embodiments, polynucleotides that are overexpressed may be targeted with siRNA, for example, and in additional specific embodiments polynucleotides that are underexpressed may be targeted with gene replacement. In either embodiment, exemplary polynucleotides of Table 3 and the sequences provided herein are employed to obtain the respective siRNA or gene replacement agents, for example.

TABLE 3
Exemplary Targeted Therapy for Polynucleotides of the Invention

| Gene | Exemplary Targeted therapy | Exemplary Targeted |
|---|---|---|
| Symbol | (published) | Therapy |
| FOS | antisense oligonucleotide, siRNA | siRNA |
| TCEAL1 | | siRNA |
| HIST1H4C | histone deacetylase inhibitor | |
| PRKAR2B | antisense oligonucleotide | active site inhibitor |
| DUSP6 | | phosphatase inhibitor |
| MXI1 | | siRNA |
| ANXA3 | | antibody, siRNA |
| AR | antiandrogens | |
| TGFB1I4 | | siRNA |
| MXI1 | | siRNA |
| IER3 | | siRNA |
| ESD | | siRNA |
| NR1D2 | | ligand antagonist |
| SOX9 | retinoic acid agonists, | |
| | proteasomal agents | |
| BCL2 | antisense oligonucleotide | |
| ARID5B | | siRNA |
| PRKRIR | | active site inhibitor |
| ZNF292 | | siRNA |
| IER2 | | siRNA |
| CD164 | antibodies | |
| DICER1 | shRNA and siRNA | |
| BTG1 | | siRNA |
| EEF1B2 | | siRNA |
| ATP2B1 | | siRNA |
| KPNB3 | | siRNA |
| RBPMS | | siRNA |
| DICER1 | | siRNA |
| BLNK | pharmacologic | |
| CDKN1B | | siRNA |
| BANF1 | | siRNA |
| TOPORS | | proteasome inhibitors |
| CCDC6 | | siRNA |
| RBMX | | siRNA |
| KIAA1354 | | siRNA |
| TLK1 | | siRNA |
| PNRC1 | | siRNA |
| ERCC4 | | siRNA |
| C14orf11 | | siRNA |
| FMR1 | | siRNA |
| SFRS3 | | siRNA |
| RPL36AL | | siRNA |
| NDUFA7 | | siRNA |
| MGC39325 | | siRNA |
| SHOC2 | | siRNA |
| TOP2B | | siRNA |
| DUT | | siRNA |
| MGC9084 | | siRNA |
| KIAA0261 | | siRNA |
| JWA | | siRNA |
| C18orf1 | | siRNA |
| KIAA0240 | | siRNA |
| CD2AP | | siRNA |
| RASSF3 | | siRNA |
| GYPC | | siRNA |
| SFRS12 | | siRNA |
| CDS2 | siRNA | |
| SP3 | | siRNA |
| MTMR3 | | phosphatase inhibitor |
| KIAA0423 | | siRNA |
| FBXO21 | | siRNA |
| KIAA0947 | | siRNA |
| TCF12 | | siRNA |
| FIBP | | siRNA |
| CSF1 | polysaccharide Krestin upregulates | |
| ST14 | antisense oligonucleotide | |
| HYOU1 | | GR (gene replacement) |
| CASP7 | | GR |
| CHPF | | Cox 2 inhibitor |
| PHLDA2 | | GR |
| ABCC5 | | GR |
| RRBP1 | | GR |
| EIF3S2 | | GR |
| PPIA | | GR |
| BTN3A2 | | GR |
| C19orf10 | | GR |
| PFKL | | GR |
| ARHGDIA | | GR |
| EVER1 | | antibody |
| IDH2 | | GR |
| MSF | | GR |
| SIAT4A | | GR |
| PKM2 | | GR |
| HSPA5 | | GR |
| SYNGR2 | | GR |
| PITX1 | | GR |
| GAGE2 | | GR |
| HLA-C | | GR |
| PYCR1 | | GR |
| STAT3 | g quartet oligo | |
| SLC9A3R1 | antiestrogen | |
| (also referred | | |
| to as EBP50) | | |
| SECTM1 | | antibody |
| CA12 | | antibody |
| EEF1A2 | | GR |
| IGFBP4 | | GR |
| IGFBP4 | | GR |
| APOD | | GR |

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents and Patent Applications

U.S. Patent Application Publication Number US 2003/0198972
U.S. Patent Application Publication Number US 2003/0236632
U.S. Patent Application Publication Number US 2003/0224374
U.S. Patent Application Publication Number US 2003/0186248
U.S. Patent Application Publication Number US 2004/0002067
U.S. Patent Application Publication Number US 2004/0058340
PCT Patent Application WO 02/103320
PCT Patent Application WO 03/060164
PCT Patent Application WO 03/060470

### Publications

Adeyinka A, Emberley E, Niu Y, Snell L, Murphy L C, Sowter H, et al. Analysis of gene expression in ductal carcinoma in situ of the breast. Clin Cancer Res 2002;8:3788-95.
Altman D G, Lausen B, Sauerbrei W, Schumacher M. Dangers of using “optimal” cutpoints in the evaluation of prognostic factors. J Natl Cancer Inst 1994;86:829-35.
Alwine J C, Kemp D J, Stark G R. Method for detection of specific RNAs in agarose gels by transfer to diazobenzyloxymethyl-paper and hybridization with DNA probes. Proc. Natl. Academy Science USA 1977;74:5350-4.
Ambroise C, McLachlan G J. Selection bias in gene extraction on the basis of microarray gene-expression data. Proc Natl Acad Sci USA 2002;99:6562-6.
Andersen C L, Monni O, Wagner U, Kononen J, Barlund M, Bucher C, et al. High-throughput copy number analysis of 17q23 in 3520 tissue specimens by fluorescence in situ hybridization to tissue microarrays. Am J Pathol 2002;161:73-9.
Anzick, S. L., Kononen, J., Walker, R. L., Azorsa, D. O., Tanner, M. M., Guan, X. Y., Sauter, G., Kallioniemi, O. P., Trent, J. M., and Meltzer, P. S. AIB1, a steroid receptor coactivator amplified in breast and ovarian cancer. Science, 277: 965-968, 1997.
Aoyagi K, Tatsuta T, Nishigaki M, Akimoto S, Tanabe C, Omoto Y, et al. A faithful method for PCR-mediated global mRNA amplification and its integration into microarray analysis on laser-captured cells. Biochem Biophys Res Commun 2003;300:915-20.
Atanaskova, N., V. G. Keshamouni, et al. (2002). “MAP kinase/estrogen receptor cross-talk enhances estrogen-mediated signaling and tumor growth but does not confer tamoxifen resistance.” Oncogene 21(25): 4000-8.
Balasenthil, S., Barnes, C. J., Rayala, S. K., and Kumar, R. Estrogen receptor activation at serine 305 is sufficient to upregulate cyclin D1 in breast cancer cells. FEBS Lett, 567: 243-247, 2004.
Bardou, V. J., Arpino, G., Elledge, R. M., Osborne, C. K., and Clark, G. M. Progesterone receptor status significantly improves outcome prediction over estrogen receptor status alone for adjuvant endocrine therapy in two large breast cancer databases. J Clin Oncol, 21: 1973-1979, 2003.
Bardou, V. J., G. Arpino, et al. (2003). “Progesterone receptor status significantly improves outcome prediction over estrogen receptor status alone for adjuvant endocrine therapy in two large breast cancer databases.” J Clin Oncol 21(10): 1973-9.
Baum M, Budzar A U, Cuzick J, Forbes J, Houghton J H, Klijn J G, et al. Anastrozole alone or in combination with tamoxifen versus tamoxifen alone for adjuvant treatment of postmenopausal women with early breast cancer: first results of the ATAC randomised trial. Lancet 2002;359:2131-9.
Baum, M., Budzar, A. U., Cuzick, J., Forbes, J., Houghton, J. H., Klijn, J. G., and Sahmoud, T. Anastrozole alone or in combination with tamoxifen versus tamoxifen alone for adjuvant treatment of postmenopausal women with early breast cancer: first results of the ATAC randomised trial. Lancet, 359: 2131-2139, 2002.
Bautista, S., H. Valles, et al. (1998). “In breast cancer, amplification of the steroid receptor coactivator gene AIB1 is correlated with estrogen and progesterone receptor positivity.” Clin Cancer Res 4(12): 2925-9.
Beatson, G. T. (1896). “On the treatment of inoperable cases of carcinogen of the mamma: suggestions for a new method of treatment with illustrative cases.” Lancet 2: 104-107, 162-167.
Benz C C, Scott G K, Sarup J C, Johnson R M, Tripathy D, Coronado E, et al. Estrogen-dependant, tamoxifen-resistant tumorigenic growth of MCF-7 cells transfected with HER2/neu. Breast Cancer Research and Treatment 1993;24:85-95.
Benz, C. C., G. K. Scott, et al. (1993). “Estrogen-dependant, tamoxifen-resistant tumorigenic growth of MCF-7 cells transfected with HER2/neu.” Breast Cancer Research and Treatment 24: 85-95.
Benz, C. C., Scott, G. K., Sarup, J. C., Johnson, R. M., Tripathy, D., Coronado, E., Shepard, H. M., and Osborne, C. K. Estrogen-dependant, tamoxifen-resistant tumorigenic growth of MCF-7 cells transfected with HER2/neu. Breast Cancer Research and Treatment, 24: 85-95, 1993.
Berk A J, Sharp P A. Sizing and mapping of early adenovirus mRNAs by gel electrophoresis of S1 endonuclease-digested hybrids. Cell 1977;12:721-32.
Bloom H, Richardson W, Harrier E. Natural history of untreated breast cancer (1805-1933). Brit Medical J 1962;2:213.
Bocquel, M. T., V. Kumar, et al. (1989). “The contribution of the N- and C-terminal regions of steroid receptors to activation of transcription is both receptor and cell-specific.” Nucleic Acids Research 17: 2581-2595.
Borg A, Ferno M, Peterson C. Predicting the future of breast cancer. Nat Med 2003;9:16-8.
Brondello, J. M., Pouyssegur, J., and McKenzie, F. R. Reduced MAP kinase phosphatase-1 degradation after p42/p44MAPK-dependent phosphorylation. Science, 286: 2514-2517, 1999.
Brzozowski, A., A. C. W. Pike, et al. (1997). “Molecular basis of agonism and antagonism in the oestrogen receptor.” Nature 389: 753-758.
Buchholz T A, Stivers D N, Stec J, Ayers M, Clark E, Bolt A, et al. Global gene expression changes during neoadjuvant chemotherapy for human breast cancer. Cancer J 2002;8:461-8.
Bunone, G., Briand, P. A., Miksicek, R. J., and Picard, D. Activation of the unliganded estrogen receptor by EGF involves the MAP kinase pathway and direct phosphorylation. Embo J, 15: 2174-2183, 1996.
Buzdar, A., J. Douma, et al. (2001). “Phase III, multicenter, double-blind, randomized study of letrozole, an aromatase inhibitor, for advanced breast cancer versus megestrol acetate.” J Clin Oncol 19(14): 3357-66.
Campbell, R. A., P. Bhat-Nakshatri, et al. (2001). “Phosphatidylinositol 3-kinase/AKT-mediated activation of estrogen receptor alpha: A new model for anti-estrogen resistance.” J Biol Chem 276: 9817-24.
Camps M, Nichols A, Arkinstall S. Dual specificity phosphatases: a gene family for control of MAP kinase function. Faseb J 2000;14:6-16.
Camps M, Nichols A, Gillieron C, Antonsson B, Muda M, Chabert C, et al. Catalytic activation of the phosphatase MKP-3 by ERK2 mitogen-activated protein kinase. Science 1998;280:1262-5.
Camps, M., Chabert, C., Muda, M., Boschert, U., Gillieron, C., and Arkinstall, S. Induction of the mitogen-activated protein kinase phosphatase MKP3 by nerve growth factor in differentiating PC12. FEBS Lett, 425: 271-276, 1998.
Camps, M., Nichols, A., and Arkinstall, S. Dual specificity phosphatases: a gene family for control of MAP kinase function. Faseb J, 14: 6-16, 2000.
Camps, M., Nichols, A., Gillieron, C., Antonsson, B., Muda, M., Chabert, C., Boschert, U., and Arkinstall, S. Catalytic activation of the phosphatase MKP-3 by ERK2 mitogen-activated protein kinase. Science, 280: 1262-1265, 1998.
Carlson, R. W. and I. C. Henderson (2003). “Sequential hormonal therapy for metastatic breast cancer after adjuvant tamoxifen or anastrozole.” Breast Cancer Res Treat 80 Suppl 1: S19-26; discussion S27-8.
Carter C, Allen C, Henson D. Relation of tumor size, lymph node status, and survival in 24,740 breast cancer cases. Cancer 1989;63:181.
Castoria, G., M. V. Barone, et al. (1999). “Non-transcriptional action of oestradiol and progestin triggers DNA synthesis.” Embo J 18(9): 2500-10.
Chang J, Powles T J, Allred D C. Biologic markers as predictors of clinical outcome from systematic therapy for primary operable breast cancer. Journal of Clinical Onocolgy 2000;18:1601-2.
Chang J, Wooten E C, Elledge R M, Hilsenbeck S, Tsimelzon A, Mohsin S, et al. Gene expression profiles for docetaxel chemosensitivity. Proc Am Society Clin Oncol 2002;21:426a.
Chang, J. C., Hilsenbeck, S. G., and Fuqua, S. A. Genomic approaches in the management and treatment of breast cancer. Br J Cancer, 92: 618-624, 2005.
Chen, D., T. Riedl, et al. (2000). “Activation of estrogen receptor alpha by S118 phosphorylation involves a ligand-dependent interaction with TFIIH and participation of CDK7.” Mol Cell 6:127-37.
Cho, H. and B. S. Katzenellenbogen (1993). “Synergistic activation of estrogen receptor-mediated transcription by estradiol and protein kinase activators.” Molecular Endocrinology 7(3): 441-452.
Chrenek M A, Wong P, Weaver V M. Tumour-stromal interactions: Integrins and cell adhesions as modulators of mammary cell survival and transformation. Breast Cancer Res 2001;3:224-9.
Clark G M. Prognostic and Predictive Factors. In Harris J R, Lippman M E, Morrow M, Osborne C K, editors. Diseases of the Breast. Vol 2. Philadelphia: Lippincott Williams & Wilkins; 2000.
Clark, G. M. and W. L. McGuire (1988). “Steroid receptors and other prognostic factors in primary breast cancer.” Seminars in Oncology 15(2 Suppl 1 (April)): 20-25.
Clark, G. M., W. L. McGuire, et al. (1983). “The importance of estrogen and progesterone receptor in primary breast cancer.” Prog Clin Biol Res 132E: 183-90.
Cooper C S. Applications of microarray technology in breast cancer research. Breast Cancer Res 2001;3:158-75.
Coser, K. R., J. Chesnes, et al. (2003). “Global analysis of ligand sensitivity of estrogen inducible and suppressible genes in MCF7/BUS breast cancer cells by DNA microarray.” Proc Natl Acad Sci USA 100(24): 13994-9.
Cui, X., Zhang, P., Deng, W., Oesterreich, S., Lu, Y., Mills, G. B., and Lee, A. V. Insulin-like growth factor-I inhibits progesterone receptor expression in breast cancer cells via the phosphatidylinositol 3-kinase/Akt/mammalian target of rapamycin pathway: progesterone receptor as a potential indicator of growth factor activity in breast cancer. Mol Endocrinol, 17: 575-588, 2003.
Cui, Y., Zhang, M., Pestell, R., Curran, E. M., Welshons, W. V., and Fuqua, S. A. W. Phosphorylation of estrogen receptor a blocks its acetylation and regulates estrogen sensitivity. Cancer Research, 64: 9199-9208, 2004.
Daidone, M. G., A. Luisi, et al. (1999). “Clinical studies of Bcl-2 and treatment benefit in breast cancer patients.” Endocr Relat Cancer 6(1): 61-8.
Deapen, D., L. Liu, et al. (2002). “Rapidly rising breast cancer incidence rates among Asian-American women.” Int J Cancer 99(5): 747-50.
Doisneau-Sixou, S. F., Sergio, C. M., Carroll, J. S., Hui, R., Musgrove, E. A., and Sutherland, R. L. Estrogen and antiestrogen regulation of cell cycle progression in breast cancer cells. Endocr Relat Cancer, 10: 179-186, 2003.
Dowsett, M. (2003). “Analysis of time to recurrence in the ATAC (armidex, tamoxifen, alone or in combination) trial according to estrogen receptor and progesterone receptor status.” Breast Cancer Research and Treatment 82 (S1): S10.
Dowsett, M. Analysis of time to recurrence in the ATAC (armidex, tamoxifen, alone or in combination) trial according to estrogen receptor and progesterone receptor status. Breast Cancer Research and Treatment, 82 (S1): S10, 2003.
Dressman M A, Walz T M, Lavedan C, Barnes L, Buchholtz S, Kwon I, et al. Genes that co-cluster with estrogen receptor alpha in microarray analysis of breast biopsies. Pharmacogenomics J 2001;1:135-41.
Driggers, P. H. and J. H. Segars (2002). “Estrogen action and cytoplasmic signaling pathways. Part II: the role of growth factors and phosphorylation in estrogen signaling.” Trends Endocrinol Metab 13(10): 422-7.
Dudoit S, Fridlyand J. A prediction-based resampling method for estimating the number of clusters in a dataset. Genome Biol 2002;3:RESEARCH0036.
Early_Breast_Cancer_Trialists'_Collaborative_Group (1998). “Tamoxifen for early breast cancer: an overview of the randomised trials.” Lancet 351: 1451-1467.
Early_Breast_Cancer_Trialists'_Collaborative_Group Tamoxifen for early breast cancer: an overview of the randomised trials. Lancet, 351: 1451-1467, 1998.
Early_Breast_Cancer_Trialists_Collaborative_Group. Polychemotherapy for early breast cancer: an overview of the randomised trials. Early Breast Cancer Trialists' Collaborative Group. Lancet 1998;352:930-42.
Early_Breast_Cancer_Trialists_Collaborative_Group. Systemic treatment of early breast cancer by hormonal, cytotoxic, or immune therapy. 133 randomised trials involving 31,000 recurrences and 24,000 deaths among 75,000 women. Early Breast Cancer Trialists' Collaborative Group. Lancet 1992;339:1-15, 71-85.
Early_Breast_Cancer_Trialists'_Collaborative_Group. Tamoxifen for early breast cancer: an overview of the randomised trials. Lancet 1998;351:1451-67.
Elledge R M, Fuqua S A W. Estrogen and Progesterone Receptors. In Harris J R, Lippman M E, Morrow M, Osborne C K, editors. Diseases of the Breast. Vol 2. Philadelphia, Pa.: Lippincott, Williams & Wilkins; 2000. p. 471-88.
Ellis M, Davis N, Coop A, Liu M, Schumaker L, Lee R Y, et al. Development and validation of a method for using breast core needle biopsies for gene expression microarray analyses. Clin Cancer Res 2002;8:1155-66.
Encarnacion C A, Ciocca D R, McGuire W L, Clark G M, Fuqua S A, Osborne C K. Measurement of steroid hormone receptors in breast cancer patients on tamoxifen. Breast Cancer Research & Treatment 1993;26:237-46.
Encarnacion, C. A., Ciocca, D. R., McGuire, W. L., Clark, G. M., Fuqua, S. A., and Osborne, C. K. Measurement of steroid hormone receptors in breast cancer patients on tamoxifen. Breast Cancer Research & Treatment, 26: 237-246, 1993.
Encarnacion, C. A., D. R. Ciocca, et al. (1993). “Measurement of steroid hormone receptors in breast cancer patients on tamoxifen.” Breast Cancer Research & Treatment 26(3): 237-46.
Faridi J, Wang L, Endemann G, Roth R A. Expression of constitutively active Akt-3 in MCF-7 breast cancer cells reverses the estrogen and tamoxifen responsivity of these cells in vivo. Clin Cancer Res 2003;9:2933-9.
Faridi, J., Wang, L., Endemann, G., and Roth, R. A. Expression of constitutively active Akt-3 in MCF-7 breast cancer cells reverses the estrogen and tamoxifen responsivity of these cells in vivo. Clin Cancer Res, 9: 2933-2939, 2003.
Felekkis, K. N., Narsimhan, R. P., Near, R., Castro, A. F., Zheng, Y., Quilliam, L. A., and Lerner, A. AND-34 activates phosphatidylinositol 3-kinase and induces anti-estrogen resistance in a SH2 and GDP exchange factor-like domain-dependent manner. Mol Cancer Res, 3: 32-41, 2005.
Fisher B, Bryant J, Wolmark N, Mamounas E, Brown A, Fisher E R, et al. Effect of preoperative chemotherapy on the outcome of women with operable breast cancer. J Clin Oncol 1998;16:2672-85.
Fisher B, Dignam J, Wolmark N, DeCillis A, Emir B, Wickerham D L, et al. Tamoxifen and chemotherapy for lymph node-negative, estrogen receptor-positive breast cancer. J Natl Cancer Inst 1997;89:1673-82.
Fisher E R, Palekar A, Rockette H, al. e. Pathologic findings from the National Surgical Adjuvant Breast Project (Protocol No. 4). V. Significance of axillary nodal micro- and macrometastasis. Cancer 1978;42:2032-8.
Fisher E R, Redmond C, Fisher B. Histologic grading of breast cancer. Pathol Ann 1980;15:239-51.
Fisher, B. J., F. E. Perera, et al. (1998). “Long-term follow-up of axillary node-positive breast cancer patients receiving adjuvant tamoxifen alone: patterns of recurrence.” Int J Radiat Oncol Biol Phys 42(1): 117-23.
Fujimoto, N. and B. S. Katzenellenbogen (1994). “Alteration in the agonist/antagonist balance of antiestrogens by activation of protein kinase A signaling pathways in breast cancer cells: Antiestrogen selectivity and promoter dependence.” Molecular Endocrinology 8: 296-304.
Fujita, T., Y. Kobayashi, et al. (2003). “Full activation of estrogen receptor alpha activation function-1 induces proliferation of breast cancer cells.” J Biol Chem 278(29): 26704-14.
Fuqua S A W, Wiltschke C, Zhang Q X, Borg A, Castles C G, Friedrichs W E, et al. A hypersensitive estrogen receptor-α mutation in premalignant breast lesions. Cancer Research 2000;60:4026-9.
Fuqua S A W. The role of estrogen receptors in breast cancer metastasis. J Mam Gland Bio Neoplasia 2002;6:407-17.
Fuqua, S. A. W. (1994). “Estrogen receptor mutagenesis and hormone resistance.” Cancer 74(3 Suppl.): 1026-1029.
Fuqua, S. A. W. (2002). “The role of estrogen receptors in breast cancer metastasis.” J Mam Gland Bio Neoplasia 6: 407-417.
Fuqua, S. A. W., C. Wiltschke, et al. (2000). “A hypersensitive estrogen receptor-a mutation in premalignant breast lesions.” Cancer Research 60(15): 4026-4029.
Fuqua, S. A. W., Wiltschke, C., Zhang, Q. X., Borg, A., Castles, C. G., Friedrichs, W. E., Hopp, T., Hilsenbeck, S., Mohsin, S., O'Connell, P., and Allred, D. C. A hypersensitive estrogen receptor-a mutation in premalignant breast lesions. Cancer Research, 60: 4026-4029, 2000.
Fuqua, S. A., R. Schiff, et al. (2003). “Estrogen receptor beta protein in human breast cancer: correlation with clinical tumor parameters.” Cancer Res 63(10): 2434-9.
Furukawa T, Sunamura M, Motoi F, Matsuno S, Horii A. Potential tumor suppressive pathway involving DUSP6/MKP-3 in pancreatic cancer. Am J Pathol 2003;162:1807-15.
Furukawa, T., Sunamura, M., Motoi, F., Matsuno, S., and Horii, A. Potential tumor suppressive pathway involving DUSP6/MKP-3 in pancreatic cancer. Am J Pathol, 162: 1807-1815, 2003.
Gee J M, Robertson J F, Ellis I O, Nicholson R1. Phosphorylation of ERK1/2 mitogen-activated protein kinase is associated with poor response to antihormonal therapy and decreased patient survival in clinical breast cancer. Int J Cancer 2001;95:247-54.
Gee, J. M., J. F. Robertson, et al. (2001). “Phosphorylation of ERK1/2 mitogen-activated protein kinase is associated with poor response to anti-hormonal therapy and decreased patient survival in clinical breast cancer.” Int J Cancer 95(4): 247-54.
Gee, J. M., Robertson, J. F., Ellis, I. O., and Nicholson, R. I. Phosphorylation of ERK1/2 mitogen-activated protein kinase is associated with poor response to anti-hormonal therapy and decreased patient survival in clinical breast cancer. Int J Cancer, 95: 247-254, 2001.
Gelbfish, G. A., A. L. Davidson, et al. (1988). “Relationship of estrogen and progesterone receptors to prognosis in breast cancer.” Ann Surg 207(1): 75-9.
Ginestier C, Charafe-Jauffret E, Bertucci F, Eisinger F, Geneix J, Bechlian D, et al. Distinct and complementary information provided by use of tissue and DNA microarrays in the study of breast tumor markers. Am J Pathol 2002;161:1223-33.
Girault, I., F. Lerebours, et al. (2003). “Expression analysis of estrogen receptor alpha coregulators in breast carcinoma: evidence that NCOR1 expression is predictive of the response to tamoxifen.” Clin Cancer Res 9(4): 1259-66.
Gompel, A., S. Somai, et al. (2000). “Hormonal regulation of apoptosis in breast cells and tissues.” Steroids 65(10-11): 593-8.
Grammer, T. C. and Blenis, J. Evidence for MEK-independent pathways regulating the prolonged activation of the ERK-MAP kinases. Oncogene, 14: 1635-1642, 1997.
Gruvberger S, Ringner M, Chen Y, Panavally S, Saal L H, Borg A, et al. Estrogen receptor status in breast cancer is associated with remarkably distinct gene expression patterns. Cancer Research 2001;61:5979-84.
Gruvberger S, Ringner M, Chen Y, Panavally S, Saal L H, Borg A, et al. Estrogen receptor status in breast cancer is associated with remarkably distinct gene expression patterns. Cancer Res 2001;61:5979-84.
Gruvberger S K, Ringner M, Eden P, Borg A, Ferno M, Peterson C, et al. Expression profiling to predict outcome in breast cancer: the influence of sample selection. Breast Cancer Res 2002;5:23-6.
Gutierrez, M. C., Detre, S., Johnston, S., Mohsin, S. K., Shou, J., Allred, D. C., Schiff, R., Osborne, C. K., and Dowsett, M. Molecular changes in tamoxifen-resistant breast cancer: relationship between estrogen receptor, HER-2, and p38 mitogen-activated protein kinase. J Clin Oncol, 23: 2469-2476, 2005.
Harris J, Hellman S. Observations on survival curve analysis with particular reference to breast cancer treatment. Cancer 1986;57:925-8.
Harvey J M. Estrogen receptor status by immunohistochemistry is superior to the ligand-binding assay for predicting response to adjuvant endocrine therapy in breast cancer. Journal of Clinical Oncology 1999;17:1474-81.
Harvey, J. M. (1999). “Estrogen receptor status by immunohistochemistry is superior to the ligand-binding assay for predicting response to adjuvant endocrine therapy in breast cancer.” Journal of Clinical Oncology 17(5): 1474-81.
Haslam S Z, Woodward T L. Tumour-stroma interactions: Reciprocal regulation of extracellular matrix proteins and ovarian steroid activity in the mammary gland. Breast Cancer Res 2001;3:365-72.
Hayashi, S. I., H. Eguchi, et al. (2003). “The expression and function of estrogen receptor alpha and beta in human breast cancer and its clinical application.” Endocr Relat Cancer 10(2): 193-202.
Hedenfalk I, Duggan D, Chen Y, Radmacher M, Bittner M, Simon R, et al. Gene-expression profiles in hereditary breast cancer. N Engl J Med 2001;344:539-48.
Hedenfalk I A, Ringner M, Trent J M, Borg A. Gene expression in inherited breast cancer. Advances in Cancer Research 2002;84:1-34.
Hilsenbeck S G, Clark G M, McGuire W L. Why do so many prognostic factors fail to pan out? Breast Cancer Res Treat 1992;22:197-206.
Hilsenbeck S G, Clark G M. Practical p-value adjustment for optimally selected cutpoints. Stat Med 1996; 15:103-12.
Hodges, L. C., J. D. Cook, et al. (2003). “Tamoxifen functions as a molecular agonist inducing cell cycle-associated genes in breast cancer cells.” Mol Cancer Res 1(4): 300-11.
Holloway, J. N., Murthy, S., and E1-Ashry, D. A cytoplasmic substrate of mitogen-activated protein kinase is responsible for estrogen receptor-alpha down-regulation in breast cancer cells: the role of nuclear factor-kappaB. Mol Endocrinol, 18: 1396-1410, 2004.
Hopp, T. A., Weiss, H. L., Hilsenbeck, S. G., Cui, Y., Allred, D. C., Horwitz, K. B., and Fuqua, S. A. Breast cancer patients with progesterone receptor PR-A-Rich tumors have poorer disease-free survival rates. Clin Cancer Res, 10: 2751-2760, 2004.
Howell, A., D. J. DeFriend, et al. (1996). “Pharrmacokinetics, pharrmacological and anti-tumour effects of the specific anti-oestrogen ICI 182780 in women with advanced breast cancer.” Br J Cancer 1996: 300-308.
Hutcheson, I. R., J. M. Knowlden, et al. (2003). “Oestrogen receptor-mediated modulation of the EGFR/MAPK pathway in tamoxifen-resistant MCF-7 cells.” Breast Cancer Res Treat 81(1): 81-93.
Iwase, H., Z. Zhang, et al. (2003). “Clinical significance of the expression of estrogen receptors alpha and beta for endocrine therapy of breast cancer.” Cancer Chemother Pharmacol 52 Suppl 1: S34-8.
Jansen, M. P., Foekens, J. A., van Staveren, I. L., Dirkzwager-Kiel, M. M., Ritstier, K., Look, M. P., Meijer-van Gelder, M. E., Sieuwerts, A. M., Portengen, H., Dorssers, L. C., Klijn, J. G., and Berns, E. M. Molecular classification of tamoxifen-resistant breast carcinomas by gene expression profiling. J Clin Oncol, 23: 732-740, 2005.
Joel, P. B., A. M. Traish, et al. (1998). “Estradiol-induced phosphorylation of serine 118 in the estrogen receptor is independent of p42/p44 mitogen-activated protein kinase.” J Biol Chem 273(21): 13317-23.
Joel, P. B., J. Smith, et al. (1998a). “pp90rsk1 regulates estrogen receptor-mediated transcription through phosphorylation of Ser-167.” Mol Cell Biol 18: 1978-84.
Joel, P. B., Traish, A. M., and Lannigan, D. A. Estradiol-induced phosphorylation of serine 118 in the estrogen receptor is independent of p42/p44 mitogen-activated protein kinase. J Biol Chem, 273: 13317-13323, 1998.
Johnston, S. R., J. Head, et al. (2003). “Integration of signal transduction inhibitors with endocrine therapy: an approach to overcoming hormone resistance in breast cancer.” Clin Cancer Res 9(1 Pt 2): 524S-32S.
Kallioniemi O P, Wagner U, Kononen J, Sauter G. Tissue microarray technology for high-throughput molecular profiling of cancer. Hum Mol Genet 2001;10:657-62.
Karnik, P. S., S. Kulkarni, et al. (1994). “Estrogen receptor mutations in tamoxifen-resistant breast cancer.” Cancer Research 54(2): 349-353.
Kato S, Endoh H, Masuhiro Y, Kitamoto T, Uchiyama S, Sasaki H, et al. Activation of the estrogen receptor through phosphorylation by mitogen-activated protein kinase. Science 1995;270:1491-4.
Kato, S., Endoh, H., Masuhiro, Y., Kitamoto, T., Uchiyama, S., Sasaki, H., Masushige, S., Gotoh, Y., Nishida, E., and Kawashima, H. Activation of the estrogen receptor through phosphorylation by mitogen-activated protein kinase. Science, 270: 1491-1494, 1995.
Kato, S., H. Endoh, et al. (1995). “Activation of the estrogen receptor through phosphorylation by mitogen-activated protein kinase.” Science 270(5241): 1491-4.
Kim H S, Song M C, Kwak I H, Park T J, Lim I K. Constitutive induction of p-Erk1/2 accompanied by reduced activities of protein phosphatases 1 and 2A and MKP3 due to reactive oxygen species during cellular senescence. J Biol Chem 2003;278:37497-510.
Kim, H. S., Song, M. C., Kwak, I. H., Park, T. J., and Lim, I. K. Constitutive induction of p-Erk1/2 accompanied by reduced activities of protein phosphatases 1 and 2A and MKP3 due to reactive oxygen species during cellular senescence. J Biol Chem, 278: 37497-37510, 2003.
Knight, W. A. I., R. B. Livingston, et al. (1977). “Estrogen receptor is an independent prognostic factor for early recurrence in breast cancer.” Cancer Research 37: 4669-4671.
Knowlden J M, Hutcheson I R, Jones H E, Madden T, Gee J M, Harper M E, et al. Elevated levels of epidermal growth factor receptor/c-erbB2 heterodimers mediate an autocrine growth regulatory pathway in tamoxifen-resistant MCF-7 cells. Endocrinology 2003;144:1032-44.
Knowiden, J. M., Hutcheson, I. R., Jones, H. E., Madden, T., Gee, J. M., Harper, M. E., Barrow, D., Wakeling, A. E., and Nicholson, R. I. Elevated levels of epidermal growth factor receptor/c-erbB2 heterodimers mediate an autocrine growth regulatory pathway in tamoxifen-resistant MCF-7 cells. Endocrinology, 144: 1032-1044, 2003.
Kononen J, Bubendorf L, Kallioniemi A, Barlund M, Schraml P, Leighton S, et al. Tissue microarrays for high-throughput molecular profiling of tumor specimens. Nat Med 1998;4:844-7.
Korsching E, Packeisen J, Agelopoulos K, Eisenacher M, Voss R, Isola J, et al. Cytogenetic alterations and cytokeratin expression patterns in breast cancer: integrating a new model of breast differentiation into cytogenetic pathways of breast carcinogenesis. Lab Invest 2002;82:1525-33.
Kraus, W. L., K. E. Weis, et al. (1995). “Inhibitory cross-talk between steroid hormone receptors: differential targeting of estrogen receptor in the repression of its transcriptional activity by agonist- and antagonist-occupied progestin receptors.” Mol Cell Biol 15(4): 1847-57.
Kuiper, G. G., E. Enmark, et al. (1996). “Cloning of a novel estrogen receptor expressed in rat prostate and ovary.” Proceeding of the National Academy of Science 93: 5925-5930.
Kumar, R., M. Mandal, et al. (1996). “Overexpression of HER2 modulates bcl-2, bcl-XL, and tamoxifen-induced apoptosis in human MCF-7 breast cancer cells.” Clin Cancer Res 2(7): 1215-9.
Kurebayashi, J., T. Otsuki, et al. (2000). “Expression levels of oestrogen receptor-alpha, estrogen receptor-beta, coactivators, and corepressors in breast cancer.” Clin Cancer Res 6: 512-8.
Kurokawa H, Lenferink A E, Simpson J F, Pisacane P I, Sliwkowski M X, Forbes J T, et al. Inhibition of HER2/neu (erB-2) and mitogen-activated protein kinase enhances tamoxifen action against HER2-overxpressing, tamoxifen-resistant breast cancer cells. Cancer Res 2000;60:5887-94.
Kurokawa, H., A. E. Lenferink, et al. (2000). “Inhibition of HER2/neu (erbB-2) and mitogen-activated protein kinases enhances tamoxifen action against HER2-overexpressing, tamoxifen-resistant breast cancer cells.” Cancer Res 60(20): 5887-94.
Kurokawa, H., Lenferink, A. E., Simpson, J. F., Pisacane, P. I., Sliwkowski, M. X., Forbes, J. T., and Arteaga, C. L. Inhibition of HER2/neu (erB-2) and mitogen-activated protein kinass enhances tamoxifen action against HER2-overxpressing, tamoxifen-resistant breast cancer cells. Cancer Res, 60: 5887-5894, 2000.
Kuukasjarvi, T., J. Kononen, et al. (1996). “Loss of estrogen receptor in recurrent breast cancer is associated with poor response to endocrine therapy.” Journal of Clinical Oncology 14: 2584.
Lamb, J., M. H. Ladha, et al. (2000). “Regulation of the functional interaction between cyclin D1 and the estrogen receptor.” Mol Cell Biol 20(23): 8667-75.
Lange, C. A., Shen, T., and Horwitz, K. B. Phosphorylation of human progesterone receptors at serine-294 by mitogen-activated protein kinase signals their degradation by the 26S proteasome. Proc Natl Acad Sci USA, 97: 1032-1037, 2000.
Larsen, S. S., Egeblad, M., Jaattela, M., and Lykkesfeldt, A. E. Acquired antiestrogen resistance in MCF-7 human breast cancer sublines is not accomplished by altered expression of receptors in the ErbB-family. Breast Cancer Res Treat, 58: 41-56, 1999.
Lavinsky, R. M., K. Jepsen, et al. (1998). “Diverse signaling pathways modulate nuclear receptor recruitment of N-CoR and SMRT complexes.” Proceedings of the National Academy of Science USA 95(6): 2920-2925.
Le Goff, P., Montano, M. M., Schodin, D. J., and Katzenellenbogen, B. S. Phosphorylation of the human estrogen receptor. Identification of hormone-regulated sites and examination of their influence on transcriptional activity. J Biol Chem, 269: 4458-4466, 1994.
Li C, Wong W H. Model-based analysis of oligonucleotide arrays: expression index computation and outlier detection. Proc Natl Acad Sci USA 2001;98:31-6.
Li, C. and Wong, W. H. Model-based analysis of oligonucleotide arrays: model validation, design issues and standard error application. Genome Biology, 2: 32.31-32.11, 2001.
Liang P, Averboukh L, Keyomarsi K, Sager R, Pardee A B. Differential display and cloning of messenger RNAs from human breast cancer versus mammary epithelial cells. Cancer Research 1992;52:6966-8.
Lin, Y. W., Chuang, S. M., and Yang, J. L. ERK1/2 achieves sustained activation by stimulating MAPK phosphatase-1 degradation via the ubiquitin-proteasome pathway. J Biol Chem, 278: 21534-21541, 2003.
Lobenhofer E K, Marks J R. Estrogen-induced mitogenesis of MCF-7 cells does not require the induction of mitogen-activated protein kinase activity. J Steroid Biochem Mol Biol 2000;75:11-20.
Lobenhofer, E. K. and Marks, J. R. Estrogen-induced mitogenesis of MCF-7 cells does not require the induction of mitogen-activated protein kinase activity. J Steroid Biochem Mol Biol, 75: 11-20, 2000.
Louie M C, Zou J X, Rabinovich A, Chen H W. ACTR/AIB1 functions as an E2F1 coactivator to promote breast cancer cell proliferation and antiestrogen resistance. Mol Cell Biol 2004;24:5157-71.
Louie, M. C., Zou, J. X., Rabinovich, A., and Chen, H. W. ACTR/AIB1 functions as an E2F1 coactivator to promote breast cancer cell proliferation and antiestrogen resistance. Mol Cell Biol, 24: 5157-5171, 2004.
Lu M, Miller K D, Gokmen-Polar Y, Jeng M H, Kinch M S. EphA2 overexpression decreases estrogen dependence and tamoxifen sensitivity. Cancer Res 2003;63:3425-9.
Lu, M., Miller, K. D., Gokmen-Polar, Y., Jeng, M. H., and Kinch, M. S. EphA2 overexpression decreases estrogen dependence and tamoxifen sensitivity. Cancer Res, 63: 3425-3429, 2003.
Ma X J, Wang Z, Ryan P D, Isakoff S J, Barmettler A, Fuller A, et al. A two-gene expression ratio predicts clinical outcome in breast cancer patients treated with tamoxifen. Cancer Cell 2004;5:607-16.
Ma, X. J., Wang, Z., Ryan, P. D., Isakoff, S. J., Barmettler, A., Fuller, A., Muir, B., Mohapatra, G., Salunga, R., Tuggle, J. T., Tran, Y., Tran, D., Tassin, A., Amon, P., Wang, W., Enright, E., Stecker, K., Estepa-Sabal, E., Smith, B., Younger, J., Balis, U., Michaelson, J., Bhan, A., Habin, K., Baer, T. M., Brugge, J., Haber, D. A., Erlander, M. G., and Sgroi, D. C. A two-gene expression ratio predicts clinical outcome in breast cancer patients treated with tamoxifen. Cancer Cell, 5: 607-616, 2004.
Mandlekar, S. and A. N. Kong (2001). “Mechanisms of tamoxifen-induced apoptosis.” Apoptosis 6(6): 469-77.
Mann, S., R. Laucirica, et al. (2001). “Estrogen receptor beta expression in invasive breast cancer.” Hum Pathol 32(1): 113-8.
Martin, M. B., T. F. Franke, et al. (2000). “A role for Akt in mediating the estrogenic functions of epidermal growth factor and insulin-like growth factor I.” Endocrinology 141(12): 4503-11.
Maskarinec, G. (2000). “Breast cancer—interaction between ethnicity and environment.” In vivo 14(1): 115-23.
Massarweh, S., J. Shou, et al. (2002). “Inhibition of Epidermal Growth Factor/HER2 Receptor Signaling Using ZD1839 (“Iressa”) Restores Tamoxifen Sensitivity and Delays Resistance to Estrogen Deprivation in HER2-Overexpressing Breast Tumors (abstract #130).” Proceeding of the American Society of Clinical Oncologists 22: 339.
McDonnell, D. P., D. L. Clemm, et al. (1995). “Analysis of estrogen receptor function in vitro reveals three distinct classes of antiestrogens.” Molecular Endocrinology 9: 659-668.
McDonnell, D. P., M. M. Shahbaz, et al. (1994). “The human progesterone receptor A-form functions as a transcriptional modulator of mineralocorticoid receptor transcriptional activity.” J Steroid Biochem Mol Biol 48(5-6): 425-32.
McGuire W L, Tandon A K, Allred D C, Chamness G C, Ravdin P M, Clark G M. Treatment decisions in axillary node-negative breast cancer patients. Journal of the National Cancer Institute. Monographs 1992:173-80.
McGuire W L. Hormone receptors: their role in predicting prognosis and response to endocrine therapy. Seminars in Oncology 1978;5:428-33.
McKenna, N. J., R. B. Lanz, et al. (1999). “Nuclear receptor coregulators: cellular and molecular biology.” Endocrine Reviews 20: 321-344.
McMahon, C. T., J. Suthiphongchai, et al. (1999). “P/CAF associates with cyclin D1 and potentiates its activation of the estrogen receptor.” Proceedings of the National Academy of Sciences of the United States of America 96: 5382-5387.
McShane LM, Radmacher M D, Freidlin B, Yu R, Li M C, Simon R. Methods for assessing reproducibility of clustering patterns observed in analyses of microarray data. Bioinformatics 2002;18:1462-9.
Michalides, R., Griekspoor, A., Balkenende, A., Verwoerd, D., Janssen, L., Jalink, K., Floore, A., Velds, A., van't Veer, L., and Neefjes, J. Tamoxifen resistance by a conformational arrest of the estrogen receptor alpha after PKA activation in breast cancer. Cancer Cell, 5: 597-605, 2004.
Migliaccio A, Di Domenico M, Castoria G, de Falco A, Bontempo P, Nola E, et al. Tyrosine kinase/p21ras/MAP-kinase pathway activation by estradiol-receptor complex in MCF-7 cells. EMBO Journal 1996;15:1292-300.
Migliaccio, A., Di Domenico, M., Castoria, G., de Falco, A., Bontempo, P., Nola, E., and Auricchio, F. Tyrosine kinase/p21ras/MAP-kinase pathway activation by estradiol-receptor complex in MCF-7 cells. EMBO Journal, 15: 1292-1300, 1996.
Misrahi, M., M. Atger, et al. (1987). “Complete amino acid sequence of the human progesterone receptor deduced from cloned cDNA.” Biochemical and Biophysical Research Communications 143(2, March 13): 740-748.
Morrison, A. J., R. E. Herrera, et al. (2003). “Dominant-negative nuclear receptor corepressor relieves transcriptional inhibition of retinoic acid receptor but does not alter the agonist/antagonist activities of the tamoxifen-bound estrogen receptor.” Mol Endocrinol 17(8): 1543-54.
Mote, P. A., J. A. Leary, et al. (2004). “Germ-line mutations in BRCA1 or BRCA2 in the normal breast are associated with altered expression of estrogen-responsive proteins and the predominance of progesterone receptor A.” Genes Chromosomes Cancer 39(3): 236-48.
Moulders S L, Yakes F M, Muthuswamy S K, Bianco R, Simpson J F, Arteaga C L. Epidermal groath factor receptor (HER1) tyrosine kinase inhibitor ZD1839 (Iressa) inhibits HER2/neu (erbB2)-overexpressing breast cancer cells in vitro and in vivo. Cancer Research 2001;61:8887-95.
Moulders, S. L., F. M. Yakes, et al. (2001). “Epidermal groath factor receptor (HER1) tyrosine kinase inhibitor ZD1839 (Iressa) inhibits HER2/neu (erbB2)-overexpressing breast cancer cells in vitro and in vivo.” Cancer Research 61: 8887-8895.
Moulders, S. L., Yakes, F. M., Muthuswamy, S. K., Bianco, R., Simpson, J. F., and Arteaga, C. L. Epidermal groath factor receptor (HER1) tyrosine kinase inhibitor ZD 1839 (Iressa) inhibits HER2/neu (erbB2)-overexpressing breast cancer cells in vitro and in vivo. Cancer Research, 61: 8887-8895, 2001.
Murphy, L. C., E. Leygue, et al. (2002). “Relationship of coregulator and oestrogen receptor isoform expression to de novo tamoxifen resistance in human breast cancer.” Br J Cancer 87(12): 1411-6.
Murphy, L. C., S. L. Simon, et al. (2000). “Altered expression of estrogen receptor coregulators during human breast tumorigenesis.” Cancer Res 60(22): 6266-71.
Murphy, L., Cherlet, T., Adeyinka, A., Niu, Y., Snell, L., and Watson, P. Phospho-serine-118 estrogen receptor-alpha detection in human breast tumors in vivo. Clin Cancer Res, 10: 1354-1359, 2004.
Nacht M, Ferguson A T, Zhang W, Petroziello J M, Cook B P, Gao Y H, et al. Combining serial analysis of gene expression and array technologies to identify genes differentially expressed in breast cancer. Cancer Res 1999;59:5464-70.
Nawaz, Z., Lonard, D. M., Dennis, A. P., Smith, C. L., and O'Malley, B. W. Proteasome-dependent degradation of the human estrogen receptor. Biochemistry, 96: 1858-1862, 1999.
Nedergaard, L., T. Haerslev, et al. (1995). “Immunohistochemical study of estrogen receptors in primary breast carcinomas and their lymph node metastases including comparison of two monoclonal antibodies.” APMIS 103(1): 20-4.
Neuman, E., M. H. Ladha, et al. (1997). “Cyclin D1 stimulation of estrogen receptor transcriptional activity independent of cdk4.” Molecular and Cellular Biology 17(9): 5338-5347.
Nichols, A., Camps, M., Gillieron, C., Chabert, C., Brunet, A., Wilsbacher, J., Cobb, M., Pouyssegur, J., Shaw, J. P., and Arkinstall, S. Substrate recognition domains within extracellular signal-regulated kinase mediate binding and catalytic activation of mitogen-activated protein kinase phosphatase-3. J Biol Chem, 275: 24613-24621, 2000.
Nicholson, R. I., J. M. Gee, et al. (2003). “The biology of antihormone failure in breast cancer.” Breast Cancer Res Treat 80 Suppl 1: S29-34; discussion S35.
O'Connell P, Pekkel V, Fuqua S, Osborne C K, Allred D C. Molecular genetic studies of early breast cancer evolution. Breast Cancer Research and Treatment 1994;32:5-12.
O'Connell P, Pekkel V, Fuqua S A W, Osborne C K, Allred D C. Analysis of loss of heterozygosity in 399 premalignant breast lesions at 15 genetic loci. Journal of the National Cancer Institute 1998;90:697-703.
Oh, A. S., Lorant, L. A., Holloway, J. N., Miller, D. L., Kern, F. G., and E1-Ashry, D. Hyperactivation of MAPK induces loss of ERalpha expression in breast cancer cells. Mol Endocrinol, 15: 1344-1359, 2001.
Omoto, Y., S. Kobayashi, et al. (2002). “Evaluation of oestrogen receptor beta wild-type and variant protein expression, and relationship with clinicopathological factors in breast cancers.” Eur J Cancer 38(3): 380-6.
Osborne C K, Coronado-Heinsohn E B, Hilsenbeck S G, McCue B L, Wakeling A E, McClelland R A. Comparison of the effects of a pure steroidal antiestrogen with those of tamoxifen in a model of human breast cancer. J Natl Cancer Inst 1995;87:746-50.
Osborne, C. K., Bardou, V., Hopp, T. A., Chamness, G. C., Hilsenbeck, S. G., Fuqua, S. A., Wong, J., Allred, D. C., Clark, G. M., and Schiff, R. Role of the estrogen receptor coactivator AIB1 (SRC-3) and HER-2/neu in tamoxifen resistance in breast cancer. J Natl Cancer Inst, 95: 353-361, 2003.
Osborne, C. K., Coronado-Heinsohn, E. B., Hilsenbeck, S. G., McCue, B. L., Wakeling, A. E., and McClelland, R. A. Comparison of the effects of a pure steroidal antiestrogen with those of tamoxifen in a model of human breast cancer. J Natl Cancer Inst, 87: 746-750, 1995.
Osborne, C. K., V. Bardou, et al. (2003). “Role of the estrogen receptor coactivator AIB1 (SRC-3) and HER-2/neu in tamoxifen resistance in breast cancer.” J Natl Cancer Inst 95(5): 353-61.
Osin P, Crook T, Powles T, Peto J, Gusterson B. Hormone status of in-situ cancer in BRCA1 and BRCA2 mutation carriers. Lancet 1998;351:1487.
Paech, K., P. Webb, et al. (1997). “Differential ligand activation of estrogen receptors ERα and ERb at AP1 sites.” Science 277(5331): 1508-10.
Paget S. The distribution of secondary growths in cancer of the breast. Lancet 1989;I:571-3.
Parra I, Hopp T A, Osborne C K, Allred D C, O'Connell P, Hilsenbeck S, et al. Differential gene expression profiles associated with breast cancer metastasis. Proc Am Assoc Can Res 2002;43:903.
Perou C M, Sørlie T, Eisen M B, Van de Rijn M, Jeffrey S S, Rees C, et al. Molecular portraits of human breast tumours. Nature 2000;406:747-52.
Porter D A, Krop I E, Nasser S, Sgroi D, Kaelin C M, Marks J R, et al. A SAGE (Serial Analysis of Gene Expression) view of breast tumor progression. Cancer Research 2001;61:5697-702.
Prall, O. W., Rogan, E. M., Musgrove, E. A., Watts, C. K., and Sutherland, R. L. c-Myc or cyclin D1 mimics estrogen effects on cyclin E-Cdk2 activation and cell cycle reentry. Mol Cell Biol, 18: 4499-4508, 1998.
Quiet C A, Ferguson D J, Weichselbaum R R, Hellman S. Natural history of node-negative breast cancer: a study of 826 patients with long-term follow-up. Journal of Clinical Oncology 1995;13:1144-51.
Ravdin, P. M., S. Green, et al. (1992). “Prognostic significance of progesterone receptor levels in estrogen receptor-positive patients with metastatic breast cancer treated with tamoxifen: results of a prospective Southwest Oncology Group study.” J Clin Oncol 10(8): 1284-91.
Razandi, M., A. Pedram, et al. (2000). “Plasma membrane estrogen receptors signal to antiapoptosis in breast cancer.” Mol Endocrinol 14(9): 1434-47.
Reid, J. F., Lusa, L., De Cecco, L., Coradini, D., Veneroni, S., Daidone, M. G., Gariboldi, M., and Pierotti, M. A. Limits of predictive models using microarray data for breast cancer clinical treatment outcome. J Natl Cancer Inst, 97: 927-930, 2005.
Richer J K, Lange C A, Manning N G, Owen G, Powell R, Horwitz K B. Convergence of progesterone with growth factor and cytokine signaling in breast cancer. Progesterone receptors regulate signal transducers and activators of transcription expression and activity. J Biol Chem 1998;273:31317-26.
Richer, J. K., Lange, C. A., Manning, N. G., Owen, G., Powell, R., and Horwitz, K. B. Convergence of progesterone with growth factor and cytokine signaling in breast cancer. Progesterone receptors regulate signal transducers and activators of transcription expression and activity. J Biol Chem, 273: 31317-31326, 1998.
Roodi, N., L. R. Bailey, et al. (1995). “Estrogen receptor gene analysis in estrogen receptor-positive and receptor-negative primary breast cancer.” Journal of the National Cancer Institute 87(6): 446-51.
Ross D T, Scherf U, Eisen M B, Perou C M, Rees C, Spellman P, et al. Systematic variation in gene expression patterns in human cancer cell lines. Nat Genet 2000;24:227-35.
Santen, R. J., R. X. Song, et al. (2002). “The role of mitogen-activated protein (MAP) kinase in breast cancer.” J Steroid Biochem Mol Biol 80(2): 239-56.
Scarff R W, Torioni H. Histological typing of breast tumors. In International histological. 2nd ed; 1968. p. 13-20.
Schwirzke M, Evtimova V, Burtscher H, Jarsch M, Tarin D, Weidle U H. Identification of metastasis-associated genes by transcriptional profiling of a pair of metastatic versus non-metastatic human mammary carcinoma cell lines. Anticancer Res 2001;21:1771-6.
Sgroi D C, Teng S, Robinson G, LeVangie R, Hudson J R, Jr., Elkahloun A G. In vivo gene expression profile analysis of human breast cancer progression. Cancer Research 1999;59:5656-61.
Shiau, A. K., D. Barstad, et al. (1998). “The structural basis of estrogen receptor/coactivator recognition and the antagonism of this interaction by tamoxifen.” Cell 95: 927-937.
Shou J, Massarweh S, Osborne C K, Wakeling A E, Ali S, Weiss H, et al. Mechanisms of tamoxifen resistance: increased estrogen receptor-HER2/neu cross-talk in ER/HER2-positive breast cancer. J Natl Cancer Inst 2004;96:926-35.
Shou, J., Massarweh, S., Osborne, C. K., Wakeling, A. E., Ali, S., Weiss, H., and Schiff, R. Mechanisms of tamoxifen resistance: increased estrogen receptor-HER2/neu cross-talk in ER/HER2-positive breast cancer. J Natl Cancer Inst, 96: 926-935, 2004.
Simon R, Radmacher M D, Dobbin K, McShane L M. Pitfalls in the use of DNA microarray data for diagnostic and prognostic classification. J Natl Cancer Inst 2003;95:14-8.
Simon, R. (1993). Design and conduct of clinical trials. Cancer: Principles & Practice of Oncology. V. T. DeVita, Jr., S. Hellman and S. A. Rosenberg. Philadelphia, J. B. Lippincott Co.
Smith, C. L., Z. Nawaz, et al. (1997). “Coactivator and corepressor regulation of the agonist/antagonist activity of the mixed antiestrogen, 4-Hydroxytamoxifen.” Molecular Endocrinology 11: 657-666.
Song, R. X., Santen, R. J., Kumar, R., Adam, L., Jeng, M. H., Masamura, S., and Yue, W. Adaptive mechanisms induced by long-term estrogen deprivation in breast cancer cells. Mol Cell Endocrinol, 193: 29-42, 2002.
Sorlie T, Perou C M, Tibshirani R, Aas T, Geisler S, Johnsen H, et al. Gene expression patterns of breast carcinomas distinguish tumor subclasses with clinical implications. Proc. Natl. Academy Science USA 2001;98:10869-74.
Soulez, M. and M. G. Parker (2001). “Identification of novel oestrogen receptor target genes in human ZR75-1 breast cancer cells by expression profiling.” J Mol Endocrinol 27(3): 259-74.
Stemmermann, G. N. (1991). “The pathology of breast cancer in Japanese women compared to other ethnic groups: a review.” Breast Cancer Res Treat 18 Suppl 1: S67-72.
Stendahl, M., A. A. Kronblad, et al. (2004). “Cyclin D1 overexpression is a negative predictive factor for tamoxifen response in postmenopausal breast cancer patients.” Br J Cancer 90(10): 1942-8.
Stendahl, M., Kronblad, A., Ryden, L., Emdin, S., Bengtsson, N. O., and Landberg, G. Cyclin D1 overexpression is a negative predictive factor for tamoxifen response in postmenopausal breast cancer patients. Br J Cancer, 90: 1942-1948, 2004.
Stonelake, P. S., P. G. Baker, et al. (1994). “Steroid receptors, pS2 and cathepsin D in early clinically node-negative breast cancer.” Eur J Cancer 30A(1): 5-11.
Sun, H., Charles, C. H., Lau, L. F., and Tonks, N. K. MKP-1 (3CH134), an immediate early gene product, is a dual specificity phosphatase that dephosphorylates MAP kinase in vivo. Cell, 75: 487-493, 1993.
The_ATAC_Trialists'_Group (2002). “Anastrozole anlone or in combination with tamoxifen versus tamoxifen alone for adjuvant treatment of postmenopausal women with early breast cancer: first results of the ATAC randomised trial.” Lancet 359(9324): 2131-2139.
Thottassery, J. V., Sun, Y., Westbrook, L., Rentz, S. S., Manuvakhova, M., Qu, Z., Samuel, S., Upshaw, R., Cunningham, A., and Kern, F. G. Prolonged extracellular signal-regulated kinase 1/2 activation during fibroblast growth factor 1- or heregulin beta1-induced antiestrogen-resistant growth of breast cancer cells is resistant to mitogen-activated protein/extracellular regulated kinase kinase inhibitors. Cancer Res, 64: 4637-4647, 2004.
Torhorst J, Bucher C, Kononen J, Haas P, Zuber M, Kochli O R, et al. Tissue microarrays for rapid linking of molecular changes to clinical endpoints. Am J Pathol 2001;159:2249-56.
Tzukerman, M. T., A. Esty, et al. (1994). “Human estrogen receptor transactivational capacity is determined by both cellular and promoter context and mediated by two functionally distinct intramolecular regions.” Molecular Endocrinology 8(1): 21-30.
van de Rijn M, Perou C M, Tibshirani R, Haas P, Kallioniemi 0, Kononen J, et al. Expression of cytokeratins 17 and 5 identifies a group of breast carcinomas with poor clinical outcome. Am J Pathol 2002;161:1991-6.
van de Vijver M J, He Y D, van't Veer L J, Dai H, Hart A A, Voskuil D W, et al. A gene-expression signature as a predictor of survival in breast cancer. N Engl J Med 2002;347:1999-2009.
van't Veer L J, Dai H, van De Vijver M J, He Y D, Hart A A, Mao M, et al. Gene expression profiling predicts clinical outcome of breast cancer. Nature 2002;415:530-6.
van't Veer L J, Dai H, van de Vijver M J, He Y D, Hart A A M, Mao M, et al. Gene expression profiling predicts clinical outcome of breast cancer. Nature 2002;415:530-6.
Vegeto, E., M. M. Shahbaz, et al. (1993). “Human progesterone receptor A form is a cell- and promoter-specific repressor of human progesterone receptor B function.” Mol Endocrinol 7(10): 1244-55.
Velculescu V E, Zhang L, Vogelstein B, Kinzler K W. Serial analysis of gene expression. Science 1995;270:484-7.
Verhoog L C, Brekelmans C T, Seynaeve C, van den Bosch L M, Dahmen G, van Geel A N, et al. Survival and tumour characteristics of breast-cancer patients with germline mutations of BRCA1. Lancet 1998;351:316-21.
Walter, P., S. Green, et al. (1985). “Cloning of the human estrogen receptor cDNA.” Proceedings of the National Academy of Science of the United States of America 82: 7889-7893.
Warmka, J. K., Mauro, L. J., and Wattenberg, E. V. Mitogen-activated protein kinase phosphatase-3 is a tumor promoter target in initiated cells that express oncogenic Ras. J Biol Chem, 279: 33085-33092, 2004.
Welch D R, Steeg P S, Rinker-Schaeffer C W. Molecular biology of breast cancer metastasis: Genetic regulation of human breast carcinoma metastasis. Breast Cancer Res 2000;2:408-16.
Wen, D. X., Y. F. Xu, et al. (1994). “The A and β forms of the human progesterone receptor operate through distinct signaling pathways within target cells.” Mol Cell Biol 14(12): 8356-64.
West M, Blanchette C, Dressman H, Huang E, Ishida S, Spang R, et al. Predicting the clinical status of human breast cancer by using gene expression profiles. Proc Natl Acad Sci USA 2001;98:11462-7.
Wijayaratne, A. L. and McDonnell, D. P. The human estrogen receptor-alpha is a ubiquitinated protein whose stability is affected differentially by agonists, antagonists, and selective estrogen receptor modulators. J Biol Chem, 276: 35684-35692, 2001.
Wilson K S, Roberts H, Leek R, Harris A L, Geradts J. Differential gene expression patterns in HER2/neu-positive and -negative breast cancer cell lines and tissues. Am J Pathol 2002;161:1171-85.
Wright, G. W. and Simon, R. M. A random variance model for detection of differential gene expression in small microarray experiments. Bioinformatics, 19: 2448-2455, 2003.
Zajchowski D A, Bartholdi M F, Gong Y, Webster L, Liu H L, Munishkin A, et al. Identification of gene expression profiles that predict the aggressive behavior of breast cancer cells. Cancer Res 2001;61:5168-78.
Zhang D, Salto-Tellez M, Putti T C, Do E, Koay E S. Reliability of tissue microarrays in detecting protein expression and gene amplification in breast cancer. Mod Pathol 2003;16:79-85.
Zhang, Q. X., A. Borg, et al. (1997). “An estrogen receptor mutant with strong hormone-independent activity from a metastatic breast cancer.” Cancer Research 57(7): 1244-1249.
Zhang, Z., H. Yamashita, et al. (2003). “Estrogen Receptor alpha Mutation (A-to-G Transition at Nucleotide 908) Is Not Found in Different Types of Breast Lesions from Japanese Women.” Breast Cancer 10(1): 70-73.
Zhao H, Hastie T, Whitfield M L, Borresen-Dale A L, Jeffrey S S. Optimization and evaluation of T7 based RNA linear amplification protocols for cDNA microarray analysis. BMC Genomics 2002;3:31.
Zhou B, Wu L, Shen K, Zhang J, Lawrence D S, Zhang Z Y. Multiple regions of MAP kinase phosphatase 3 are involved in its recognition and activation by ERK2. J Biol Chem 2001;276:6506-15.
Zhou, B., Wu, L., Shen, K., Zhang, J., Lawrence, D. S., and Zhang, Z. Y. Multiple regions of MAP kinase phosphatase 3 are involved in its recognition and activation by ERK2. J Biol Chem, 276: 6506-6515, 2001.
Zhou, Q., T. Hopp, et al. (2001). “Cyclin D1 in breast premalignancy and early breast cancer: Implications for prevention and treatment.” Cancer Lett 162(1): 3-17.
Zwijsen, R. M. L., E. Wientjens, et al. (1997). “CDK-independent activation of estrogen receptor by cyclin D1.” Cell 88: 405-415.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of predicting the response of an individual to a chemotherapy, comprising the steps of:
providing the level of one or more expressed polynucleotides from an individual on chemotherapy, said level from tumors that grow during or after the chemotherapy; and
comparing the level of one or more expressed polynucleotides to a control, wherein a difference between the level of at least one expressed polynucleotide predicts resistance to chemotherapy in the individual.

2. The method of claim 1, wherein the difference between the levels is defined as being higher in the individual than the control, as being lower in the individual than the control, or a combination of expressed polynucleotides being higher or lower in the individual compared to the control.

3. The method of claim 1, wherein the difference between the level of at least one expressed polynucleotide in the individual and the control is greater than one-fold.

4. The method of claim 1, wherein providing the level of the expressed polynucleotides is further defined as providing the level of expressed RNAs.

5. The method of claim 1, wherein providing the level of the expressed polynucleotides is further defined as providing the level of expressed proteins.

6. The method of claim 4, wherein providing the level of RNAs from tumors that grow during the chemotherapy comprises the following steps:
obtaining one or more cells from tumors that grow during or after the chemotherapy;
isolating RNA from the one or more cells; and
determining the level of one or more of the RNAs.

7. The method of claim 6, wherein the RNA levels are determined by microarray analysis, quantitative polymerase chain reaction, or both.

8. The method of claim 1, wherein the tumors that grow during or after the chemotherapy occurred within about one month to about five years from initiation of the chemotherapy.

9. The method of claim 1, wherein the individual has breast cancer.

10. The method of claim 1, wherein the chemotherapy is further defined as a hormone therapy.

11. The method of claim 1, wherein the hormone therapy comprises tamoxifen.

12. The method of claim 1, wherein the polynucleotides are expressed from one or more polynucleotides listed in Table 1, Table 2, or both.

13. The method of claim 1, wherein one of the expressed polynucleotides comprises DUSP6.

14. The method of claim 1, wherein when the method predicts the cancer as resistant to the chemotherapy, the individual is subjected to an alternative cancer treatment.

15. The method of claim 14, wherein the alternative cancer treatment comprises chemotherapy, radiation, surgery, gene therapy, immunotherapy, hormone therapy, or a combination thereof.

16. The method of claim 15, wherein the alternative cancer treatment being chemotherapy comprises an aromatase inhibitor, Iressa, raloxifene, ZD1839, trastuzumab, letrozole, an agent that targets the HER-2 receptor, or a combination thereof.

17. As a composition of matter, isolated expressed polynucleotides the levels of which are indicative of resistance to a chemotherapy, wherein one or more of the expressed polynucleotides are listed in Table 1, Table 2, or both.

18. The composition of claim 17, wherein the expressed polynucleotides are comprised on a substrate.

19. The composition of claim 18, wherein the substrate comprises a microarray chip.

20. The composition of claim 17, wherein the chemotherapy comprises tamoxifen.

21. As a composition of matter, a breast cancer RNA expression profile comprising DUSP6, EBP50, RhoGDIa, or a combination thereof.

22. The composition of claim 21, wherein the level of DUSP6 is indicative of resistance to tamoxifen.

23. The composition of claim 21, wherein the level of EBP50 is indicative of resistance to tamoxifen.

24. The composition of claim 21, wherein the level of RhoGDIa is indicative of resistance to tamoxifen.

25. A method of determining resistance to a chemotherapy in the cancer of an individual, comprising the step of identifying the expression level of DUSP6, EBP50, RhoGDIa, or a combination thereof in one or more cancer cells in the individual.

26. The method of claim 25, wherein the chemotherapy comprises tamoxifen.

27. The method of claim 25, further defined as comparing the level of DUSP6, EBP50, and/or RhoGDIa, respectively, in one or more cancer cells of the individual with the level of DUSP6, EBP50, and/or RhoGDIa, respectively, from one or more cells that are sensitive to the chemotherapy.

28. The method of claim 25, wherein when the level in the one or more cancer cells of the individual is higher than the level in one or more cells that are sensitive to the chemotherapy, a cancer of the individual is resistant to the chemotherapy.

29. The method of claim 25, wherein the identifying step comprises identifying an expressed DUSP6 EBP50, and/or RhoGDIa RNA level, respectively; an expressed DUSP6, EBP50, and/or RhoGDIa protein level, respectively; or both.

30. The method of claim 29, wherein the identifying step is further defined as comprising microarray analysis.

31. The method of claim 25, wherein when the cancer is resistant to the chemotherapy, the method further comprises subjecting the individual to an alternative cancer treatment.

32. The method of claim 31, wherein the alternative cancer treatment comprises chemotherapy, radiation, surgery, immunotherapy, hormone therapy, gene therapy, or a combination thereof.
